(19) United States
(12) Patent Application Publication
(10) Pub. No.: US 2016/0343958 A1
(43) Pub. Date: Nov. 24, 2016

(54) ORGANIC TRANSISTOR, COMPOUND, ORGANIC SEMICONDUCTOR MATERIAL FOR NON-LIGHT-EMITTING ORGANIC SEMICONDUCTOR DEVICE, MATERIAL FOR ORGANIC TRANSISTOR, COATING SOLUTION FOR NON-LIGHT-EMITTING ORGANIC SEMICONDUCTOR DEVICE, AND ORGANIC SEMICONDUCTOR FILM FOR NON-LIGHT-EMITTING ORGANIC SEMICONDUCTOR DEVICE

(71) Applicant: FUJIFILM Corporation, Tokyo (JP)

(72) Inventors: Yuki HIRAI, Kanagawa (JP); Kensuke MASUI, Kanagawa (JP)

(73) Assignee: FUJIFILM Corporation, Tokyo (JP)

(21) Appl. No.: 15/226,388

(22) Filed: Aug. 2, 2016

Related U.S. Application Data
(63) Continuation of application No. PCT/JP2015/052604, filed on Jan. 30, 2015.

(30) Foreign Application Priority Data
Feb. 6, 2014 (JP) 2014-021329

(51) Int. Cl. H01L 51/00; C07D 495/04; C07D 493/04; C09D 5/24

(52) U.S. Cl. CPC H01L 51/0074; C09D 5/24; C07D 495/04; H01L 51/0068; H01L 51/0073; C07D 493/04; H01L 51/0545

(57) ABSTRACT

Provided are an organic transistor containing a compound represented by the following formula, results in high carrier mobility when being used in a semiconductor active layer of the organic transistor, and exhibits high solubility in an organic solvent, in a semiconductor active layer. X is O, S, or Se; p and q are 0 to 2; R¹ to R¹⁰, R^{a}, and R^{b} are hydrogen, halogen, or -L-R; one of R¹ to R¹⁰, R^{a}, and R^{b} is -L-R; L is a specific divalent linking group; and R is an alkyl group, a cyano group, etc.

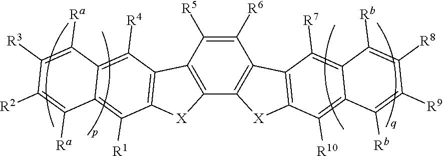

### CROSS-REFERENCE TO RELATED APPLICATIONS

This application is a Continuation of PCT International Application No. PCT/JP2015/052604, filed on Jan. 30, 2015, which claims priority under 35 U.S.C. Section 119(a) to Japanese Patent Application No. 2014-021329 filed on Feb. 6, 2014. Each of the above applications is hereby expressly incorporated by reference, in its entirety, into the present application.

### BACKGROUND OF THE INVENTION

1. Field of the Invention

The present invention relates to an organic transistor, a compound, an organic semiconductor material for a non-light-emitting organic semiconductor device, a material for an organic transistor, a coating solution for a non-light-emitting organic semiconductor device, an organic semiconductor film for a non-light-emitting organic semiconductor device, and the like. Specifically, the present invention relates to a compound having a condensed cyclic skeleton including at least two heterocyclic rings, a compound which is an intermediate compound of the above compound, an organic transistor, an organic semiconductor material for a non-light-emitting organic semiconductor device, a material for an organic transistor, a coating solution for a non-light-emitting organic semiconductor device, and an organic semiconductor film for a non-light-emitting organic semiconductor device.

2. Description of the Related Art

The devices using organic semiconductor materials are drawing great attention because they are expected to be superior in various aspects to the devices using inorganic semiconductor materials of the related art such as silicon. Examples of the devices using organic semiconductor materials include a photoelectric conversion element such as an organic thin film solar cell or a solid-state imaging element using organic semiconductor materials as photoelectric conversion materials, an organic transistor (referred to as an organic thin-film transistor in some cases) having non-light-emitting properties (in the present specification, “non-light-emitting” refers to properties by which a luminous efficiency of equal to or less than 1 lm/W is obtained in a case where electric currents are applied to a device at a current density of 0.1 mW/cm² at room temperature in the atmosphere; non-light-emitting organic semiconductor devices mean organic semiconductor devices excluding light-emitting organic semiconductor devices such as organic electroluminescence elements), and the like. The devices using organic semiconductor materials are likely to make it possible to prepare large area elements at lower temperature and lower costs compared to the devices using inorganic semiconductor materials. Furthermore, the characteristics of the materials can be easily changed by varying the molecular structure thereof. Therefore, the materials show a wide variation and can realize functions or elements that cannot be obtained by inorganic semiconductor materials.

For example, EP2251342A1 describes that if a polymer using a low-molecular weight compound such as a benzobisbenzothiophene derivative is used in an organic transistor, sufficient charge transport properties can be exhibited, and the compound exhibits excellent solubility in a solvent. However, EP2251342A1 describes neither the specific structure of the low-molecular weight compound such as a benzobisbenzothiophene derivative nor the intermediate thereof.

EP2301926A1 and EP2301921A1 describe that if a benzobisbenzothiophene derivative or the like is used as a material of an organic electro-luminescence (organic EL) element, luminescence efficiency becomes high, and the element has long service life. However, in EP2301926A1 and EP2301921A1, an investigation into the application of the benzobisbenzothiophene derivative or the like to an organic transistor is not carried out.

KR10-2012-0120886A describes that if a benzobisbenzothiophene derivative or the like is used in an organic EL element or an organic photoelectric conversion element, luminescence efficiency becomes high, driving voltage is lowered, and color is excellently developed. However, in KR10-2012-0120886A, an investigation into the application of the benzobisbenzothiophene derivative or the like to an organic transistor is not carried out.

Tetrahedron Lett., 1973 (17), 1561 discloses dimethyl-substituted benzobisbenzothiophene. However, in the document, only the generation of the compound by a photoreaction is confirmed, and the application of the compound to an organic transistor is not described.

Rao, Tilak, Journal of Scientific and Industrial Research, 1958, vol. 17 B, pp. 260-265 and Kudo, Hirotaka; Tedjamulia, Marvin L.; Castle, Raymond N.; Lee, Milton L., Journal of Heterocyclic Chemistry, 1984, vol. 21, pp. 185-192 disclose unsubstituted benzobisbenzothiophene.

### SUMMARY OF THE INVENTION

Being useful as an organic EL element material does not mean being useful as a semiconductor material for an organic transistor, because the characteristics required for the organic compound vary between the organic EL element and the organic transistor. In the organic EL element, charges need to be transported in the film thickness direction (generally, several nanometers to hundreds of nanometers) of a general film. In contrast, in the organic transistor, charges (carriers) need to be transported a long distance between electrodes (generally, several micrometers to hundreds of micrometers) in the film plane direction, and hence extremely high carrier mobility is required. Accordingly, as the semiconductor material for an organic transistor, an organic compound showing highly ordered molecular arrangement and having high crystallinity is required. Furthermore, for the expression of high carrier mobility, the n-conjugate plane thereof is preferably upright against a substrate. On the other hand, for the organic EL element, in order to improve the luminous efficiency, an element having high luminous efficiency and uniformly emitting light in plane is required. Generally, an organic compound having high crystallinity causes luminescence defectiveness such as nonuniform in-plane electric field intensity, nonuniform luminescence, and quenching of luminescence. Therefore, as the material for an organic EL element, those having low crystallinity but having high amorphousness are desirable. Consequently, the use of the organic compound constituting the organic EL element material as an organic semiconductor material does not ensure that excellent transistor characteristics can be obtained.

In addition, likewise, being useful as an organic photoelectric conversion element does not mean being useful as a semiconductor material for an organic transistor for which extremely high carrier mobility is required.

Under the circumstances described above, the inventors of the present invention conducted an investigation into the use of the compounds described in the aforementioned documents into a semiconductor active layer of an organic transistor. As a result, the inventors found that the compounds show low carrier mobility in most cases, exhibit low solubility in an organic solvent, and do not enable the formation of the semiconductor active layer of the organic transistor by a solution film formation method.

Therefore, in order to solve the aforementioned problems of the related art, the inventors of the present invention continued the investigation. An object of the present invention is to provide an organic transistor using a compound which results in high carrier mobility when being used in a semiconductor active layer of an organic transistor and exhibits high solubility in an organic solvent.

As a result of conducting thorough investigation for achieving the above objects, the inventors of the present invention obtained knowledge that by investigating various types of substituents for a condensed cyclic skeleton, that is, a mother nucleus, substituting the mother nucleus with at least one of the substituents represented by Formula (W) in particular, and limiting the substituents to specific substituents in a case where substituents (R⁵ and R⁶ in Formula (1) of the present invention) are present in a short axis direction of a molecule, a compound is obtained which results in high carrier mobility when being used in a semiconductor active layer of an organic transistor and exhibits high solubility in an organic solvent. Based on the knowledge, the inventors accomplished the present invention.

The present invention as specific means for achieving the aforementioned object is constituted as below.

[1] An organic transistor comprising a compound represented by the following Formula (1) in a semiconductor active layer:

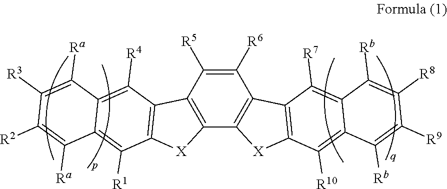

in Formula (1),

each X independently represents an O atom, a S atom, or a Se atom,

each of p and q independently represents an integer of 0 to 2,

each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, or R^{b} is a substituent represented by Formula (W), and in a case where at least one of R⁵ or R⁶ is a substituent represented by Formula (W), L in Formula (W) represented by R⁵ and R⁶ is a divalent linking group represented by the following Formula (L-2) or (L-3);

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

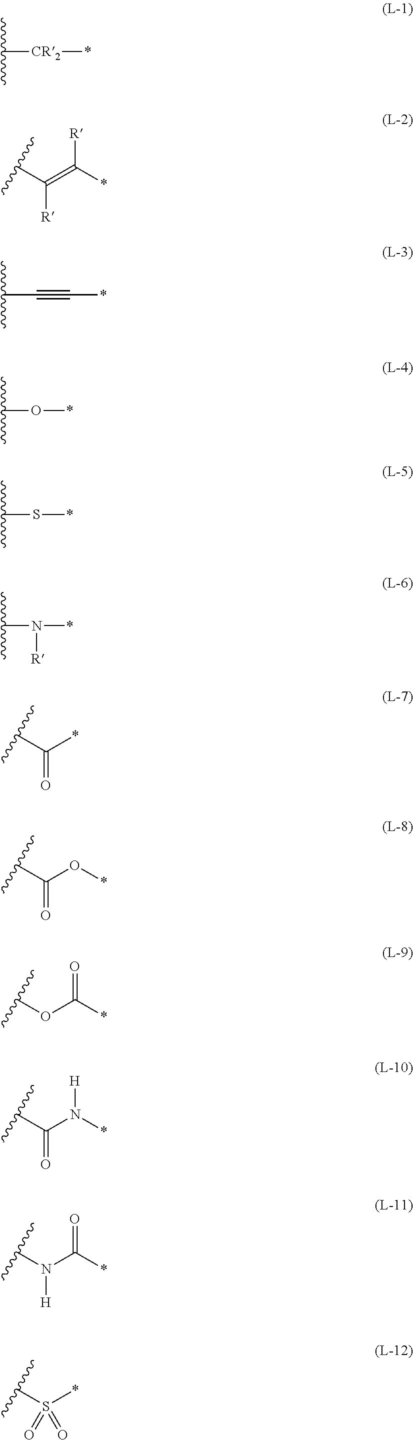

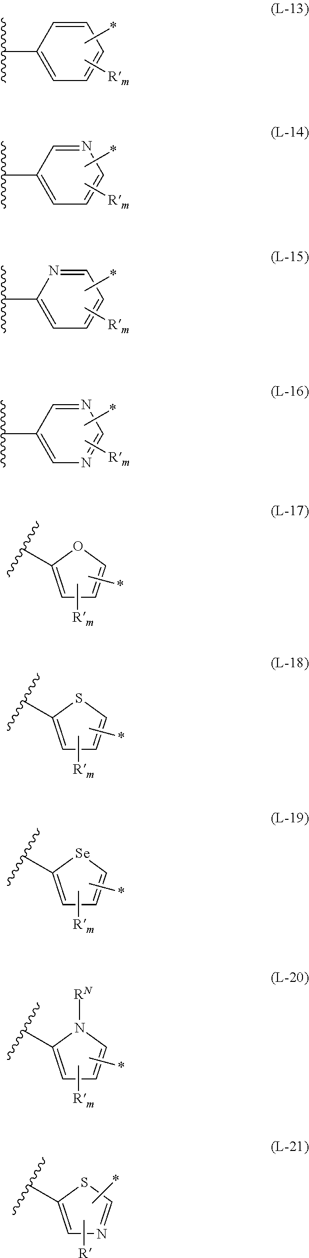

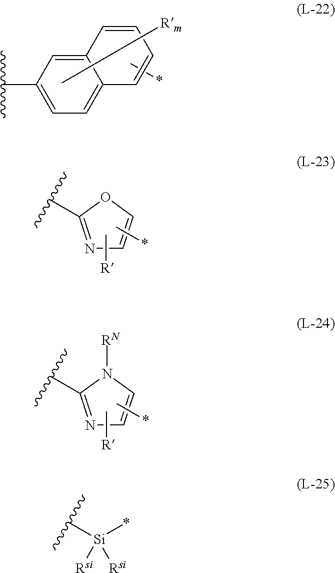

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

[2] The organic transistor described in [1], in which in Formula (1), each of R¹, R⁴ to R⁷, R¹⁰, R^{a}, and R^{b} is preferably independently a hydrogen atom or a halogen atom,

each of R², R³, R⁸, and R⁹ is preferably independently a hydrogen atom, a halogen atom, or a substituent represented by Formula (W), and at least one of R², R³, R⁸, or R⁹ is preferably a substituent represented by Formula (W).

[3] The organic transistor described in [1] or [2], in which in Formula (1), each of p and q is preferably independently 0 or 1.

[4] The organic transistor described in any one of [1] to [3], in which in Formula (1), p and q preferably satisfy p=q=0 or p=q=1.

[5] The organic transistor described in any one of [1] to [4], in which the compound represented by Formula (1) is preferably a compound represented by the following Formula (2-1):

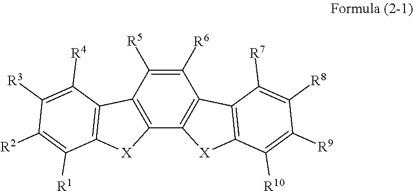

in Formula (2-1),

each X independently represents an O atom, a S atom, or a Se atom,

each of R¹, R³ to R⁸, and R¹⁰ independently represents a hydrogen atom or a halogen atom,

each of R² and R⁹ is independently a hydrogen atom, a halogen atom, or a substituent represented by the following Formula (W), at least one of R² or R⁹ is a substituent represented by Formula (W), and in a case where a substituent represented by Formula (W) is an alkyl group, the substituent represented by Formula (W) is limited to a linear alkyl group having 4 to 18 carbon atoms or a branched alkyl group having 4 or more carbon atoms,

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

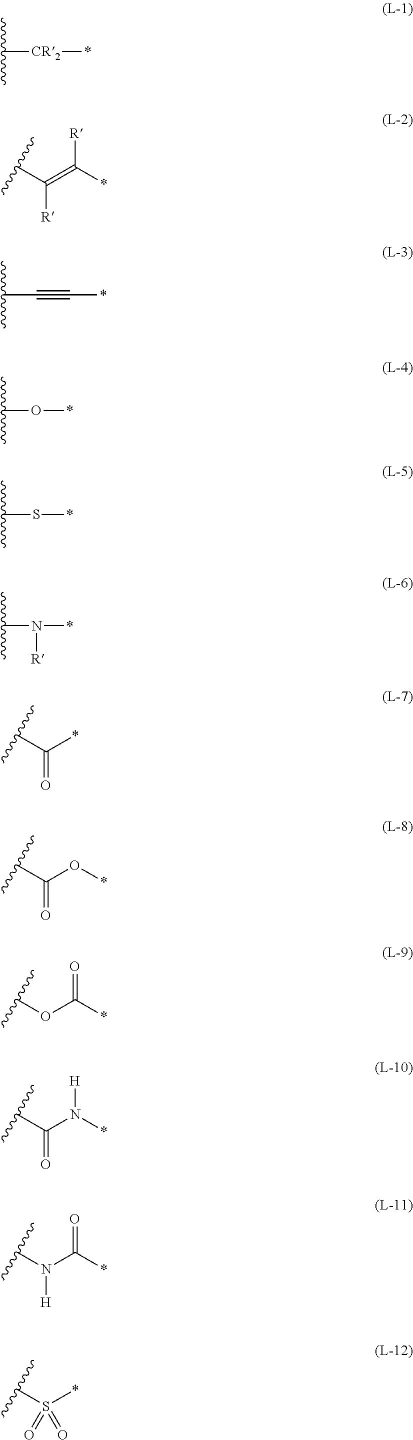

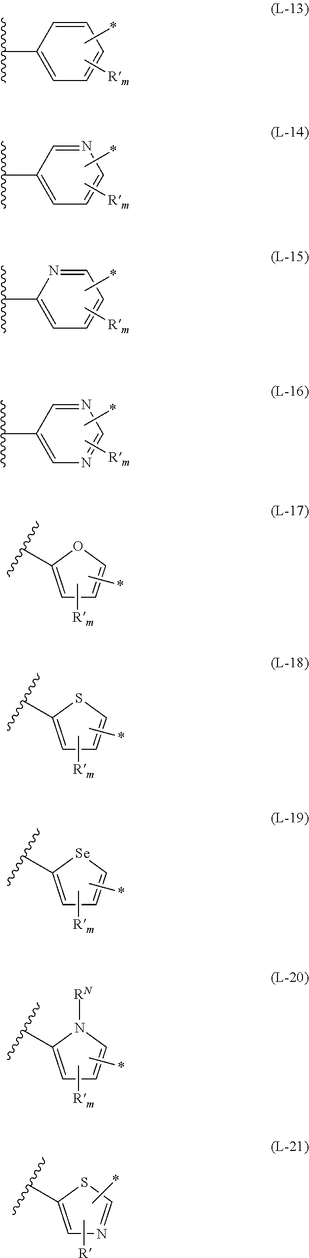

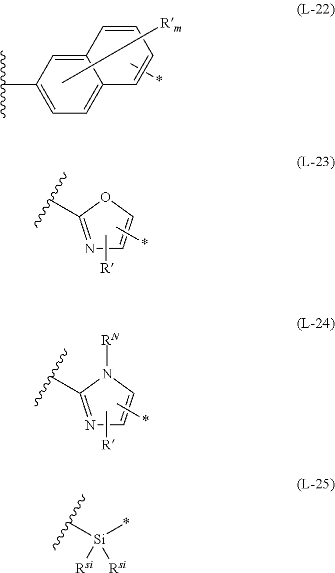

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

[6] The organic transistor described in any one of [1] to [4], in which the compound represented by Formula (1) is preferably a compound represented by the following Formula (2-2):

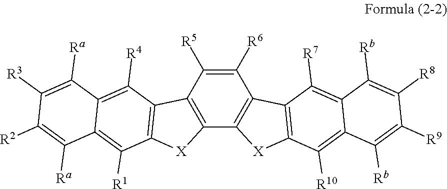

in Formula (2-2),

each X independently represents an O atom, a S atom, or a Se atom,

each of R¹, R⁴ to R⁷, R¹⁰, R^{a}, and R^{b} is independently a hydrogen atom or a halogen atom,

each of R², R³, R⁸, and R⁹ independently represents a hydrogen atom, a halogen atom, or a substituent represented by the following Formula (W), and at least one of R², R³, R⁸, or R⁹ represents a substituent represented by Formula (W).

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

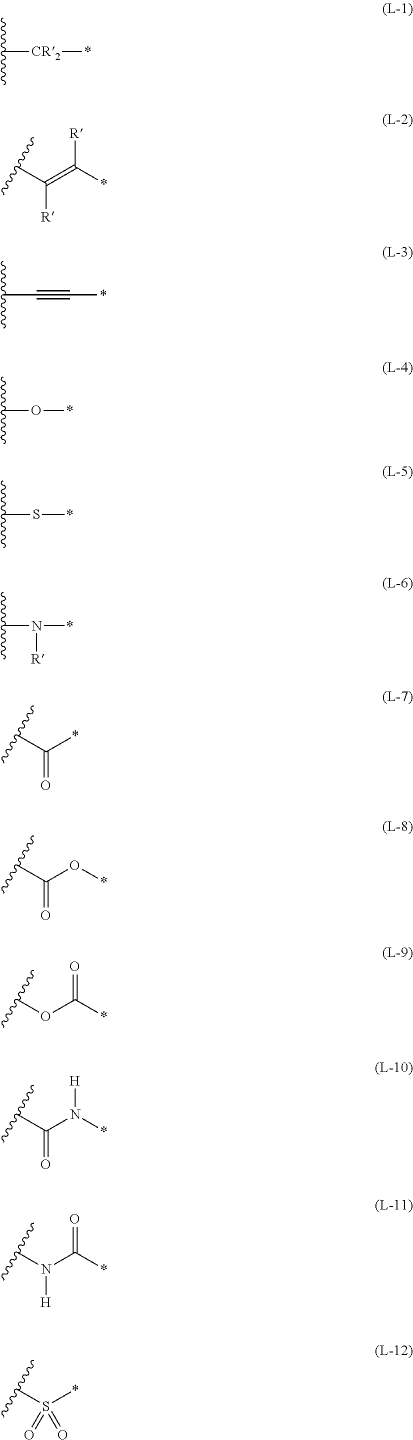

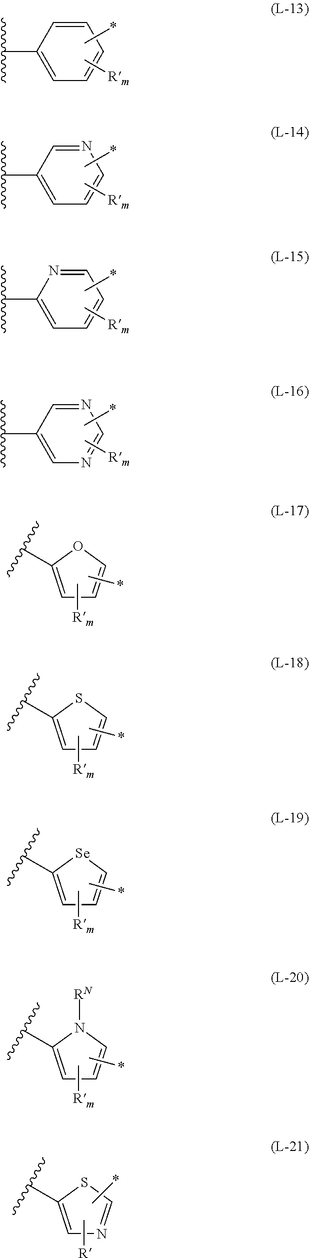

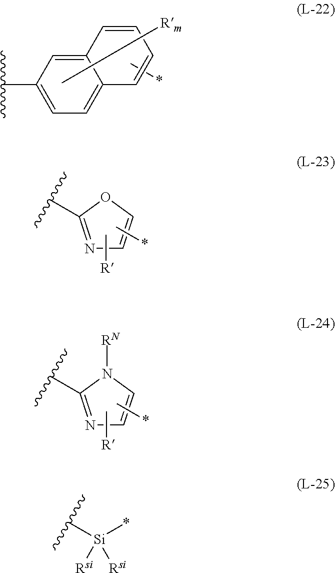

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

[7] The organic transistor described in any one of [1] to [6], in which in Formulae (1), (2-1), and (2-2), X is preferably a S atom.

[8] The organic transistor described in any one of [1] to [7], in which in Formulae (1), (2-1), and (2-2), both of R² and R⁹ are preferably a substituent represented by Formula (W), and both of R³ and R⁸ are preferably a hydrogen atom or a halogen atom, or

both of R³ and R⁸ are preferably a substituent represented by Formula (W), and both of R² and R⁹ are preferably a hydrogen atom or a halogen atom.

[9] The organic transistor described in any one of [1] to [8], in which both of R² and R⁹ are a substituent represented by Formula (W), and both of R³ and R⁸ are preferably a hydrogen atom.

[10] The organic transistor described in any one of [1] to [9], in which the total number of carbon atoms contained in a group represented by Formula (W) is preferably 4 to 18.

[11] The organic transistor described in any one of [1] to [10], in which in Formula (W), L is preferably a divalent linking group represented by any one of Formulae (L-1) to (L-4) or a divalent linking group in which a divalent linking group represented by any one of Formulae (L-2) to (L-4) is bonded to a divalent linking group represented by Formula (L-1).

[12] The organic transistor described in any one of [1] to [11], in which in Formula (W), L is preferably a divalent linking group represented by Formula (L-1), and R is preferably a substituted or unsubstituted alkyl group.

[13] A compound represented by the following Formula (2-1):

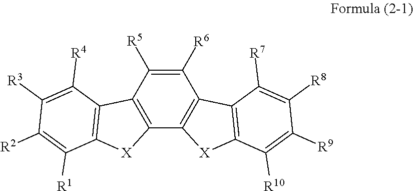

in Formula (2-1),

each X independently represents an O atom, a S atom, or a Se atom,

each of R¹, R³ to R⁸, and R¹⁰ independently represents a hydrogen atom or a halogen atom,

each of R² and R⁹ is independently a hydrogen atom, a halogen atom, or a substituent represented by the following Formula (W), at least one of R² or R⁹ is a substituent represented by Formula (W), and in a case where a substituent represented by Formula (W) is an alkyl group, the substituent represented by Formula (W) is limited to a linear alkyl group having 4 to 18 carbon atoms or a branched alkyl group having 4 or more carbon atoms.

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

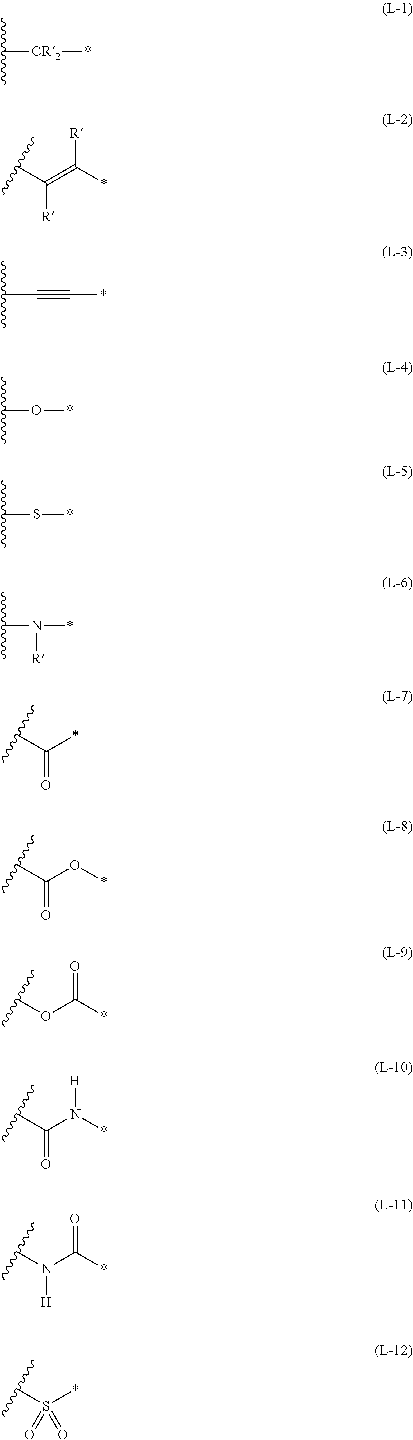

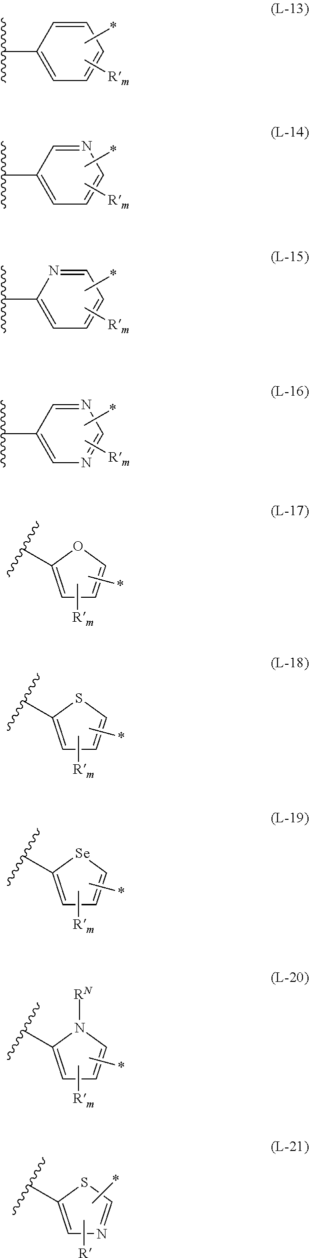

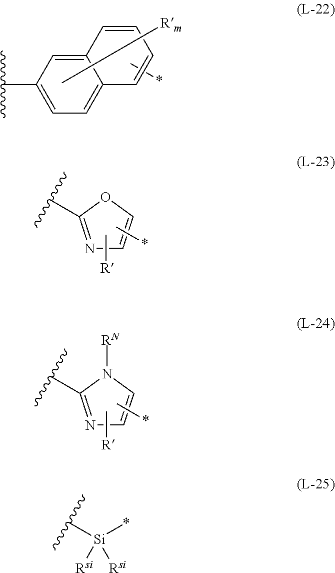

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

[14] A compound represented by the following Formula (2-2):

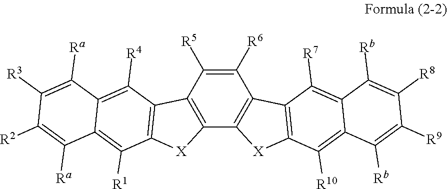

in Formula (2-2),

each X independently represents an O atom, a S atom, or a Se atom,

each of R¹, R⁴ to R⁷, R¹⁰, R^{a}, and R^{b} is independently a hydrogen atom or a halogen atom,

each of R², R³, R⁸, and R⁹ independently represents a hydrogen atom, a halogen atom, or a substituent represented by the following Formula (W), and at least one of R², R³, R⁸, or R⁹ represents a substituent represented by Formula (W).

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

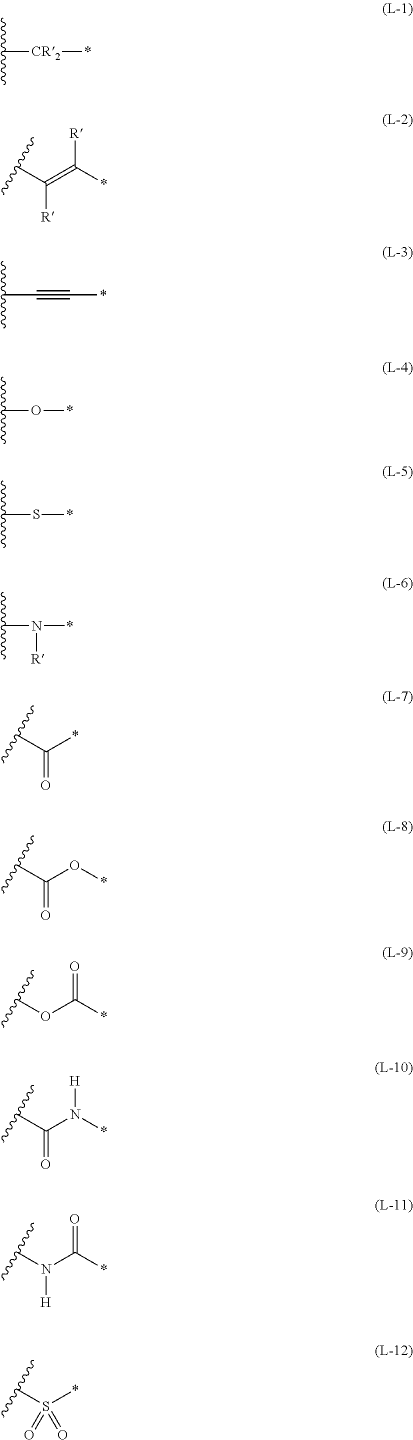

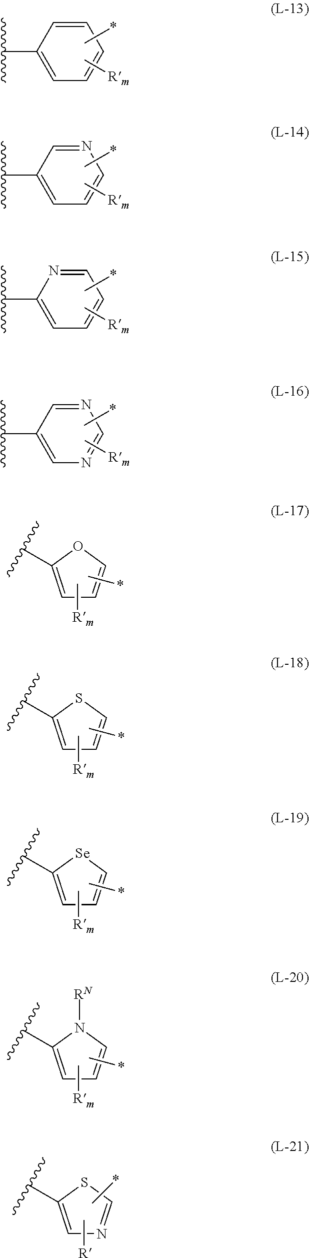

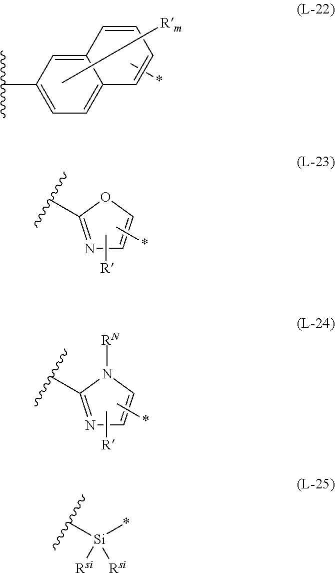

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

[15] The compound described in [13] or [14], in which in Formulae (2-1) and (2-2), X is preferably a S atom.

[16] The compound described in any one of [13] to [15], in which in Formulae (2-1) and (2-2), both of R² and R⁹ are preferably a substituent represented by Formula (W), and both of R³ and R⁸ are preferably a hydrogen atom or a halogen atom, or

both of R³ and R⁸ are preferably a substituent represented by Formula (W), and both of R² and R⁹ are preferably a hydrogen atom or a halogen atom.

[17] The compound described in any one of [13] to [16], in which both of R² and R⁹ are preferably a substituent represented by Formula (W), and both of R³ and R⁸ are preferably a hydrogen atom.

[18] The compound described in any one of [13] to [17], in which the total number of carbon atoms contained in a group represented by Formula (W) is preferably 4 to 18.

[19] The compound described in any one of [13] to [18], in which in Formula (W), L is preferably a divalent linking group represented by any one of Formulae (L-1) to (L-4) or a divalent linking group in which a divalent linking group represented by any one of Formulae (L-2) to (L-4) is bonded to a divalent linking group represented by Formula (L-1).

[20] The compound described in any one of [13] to [19], in which in Formula (W), L is preferably a divalent linking group represented by Formula (L-1), and R is preferably a substituted or unsubstituted alkyl group.

[21] An organic semiconductor material for a non-light-emitting organic semiconductor device, comprising the compound represented by Formula (1) described in any one of [1] to [12].

[22] A material for an organic transistor, comprising the compound represented by Formula (1) described in any one of [1] to [12].

[23] A coating solution for a non-light-emitting organic semiconductor device, comprising the compound represented by Formula (1) described in any one of [1] to [12].

[24] A coating solution for a non-light-emitting organic semiconductor device, comprising the compound represented by Formula (1) described in any one of [1] to [12] and a polymer binder.

[25] An organic semiconductor film for a non-light-emitting organic semiconductor device, comprising the compound represented by Formula (1) described in any one of [1] to [12].

[26] An organic semiconductor film for a non-light-emitting organic semiconductor device, comprising the compound represented by Formula (1) described in any one of [1] to [12] and a polymer binder.

[27] The organic semiconductor film for a non-light-emitting organic semiconductor device described in [25] or [26] that is preferably prepared by a solution coating method.

[28] A compound represented by the following Formula (M1):

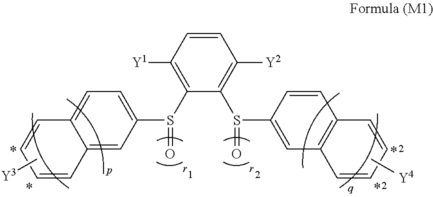

in Formula (M1),

each of Y¹ to Y⁴ independently represents a halogen atom, an alkoxy group, an acyloxy group, a hydroxy group, or a sulfonyl group,

each of * and *2 independently represents a position in which each of Y³ and Y⁴ can be substituted,

each of p and q independently represents an integer of 0 to 2, and

each of r1 and r2 independently represents an integer of 0 to 2.

[28-1] The compound described in [28], in which in the Formula (M1), each of Y¹ and Y² is preferably independently a bromine atom or an iodine atom, and each of Y³ and Y⁴ is preferably independently a chlorine atom or an alkoxy group.

[29] A compound represented by the following Formula (M2):

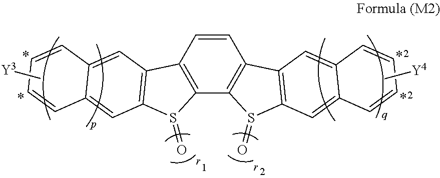

in Formula (M2),

each of Y³ and Y⁴ independently represents a halogen atom, an alkoxy group, an acyloxy group, a hydroxy group, or a sulfonyl group,

each of * and *2 independently represents a position in which each of Y³ and Y⁴ can be substituted,

each of p and q independently represents an integer of 0 to 2, and

each of r1 and r2 independently represents an integer of 0 to 2.

[29-1] The compound described in [29], in which in the Formula (M2), each of Y³ and Y⁴ is preferably independently a chlorine atom, an alkoxy group, a hydroxy group, or a sulfonyl group.

[30] The compound described in any one of [28], [28-1], [29], and [29-1] that is preferably an intermediate compound of the compound represented by Formula (1) described in any one of [1] to [12].

According to the present invention, it is possible to provide an organic transistor using a compound which results in high carrier mobility when being used in a semiconductor active layer of the organic transistor and exhibits high solubility in an organic solvent.

### BRIEF DESCRIPTION OF THE DRAWINGS

FIG. 1 is a schematic view showing a section of an exemplary structure of an organic transistor of the present invention.

FIG. 2 is a schematic view showing a section of a structure of the organic transistor manufactured as a substrate for measuring FET characteristics in examples of the present invention.

### DESCRIPTION OF THE PREFERRED EMBODIMENTS

Hereinafter, the present invention will be specifically described. The constituents described below will be explained based on representative embodiments or specific examples, but the present invention is not limited to the embodiments. In the present specification, a range of numerical values described using “to” means a range including the numerical values listed before and after “to” as a lower limit and an upper limit respectively.

In the present invention, unless otherwise specified, a hydrogen atom used in the description of each formula represents a hydrogen atom including an isotope (deuterium atom or the like). Furthermore, an atom constituting a substituent represents an atom including an isotope thereof.

[Organic Transistor]

An organic transistor of the present invention contains a compound represented by the following Formula (1) in a semiconductor active layer.

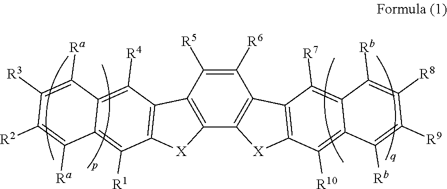

in Formula (1),

each X independently represents an O atom, a S atom, or a Se atom,

each of p and q independently represents an integer of 0 to 2,

each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, or R^{b} is a substituent represented by Formula (W), and in a case where at least one of R⁵ or R⁶ is a substituent represented by Formula (W), L in Formula (W) represented by R⁵ and R⁶ is a divalent linking group represented by the following Formula (L-2) or (L-3);

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

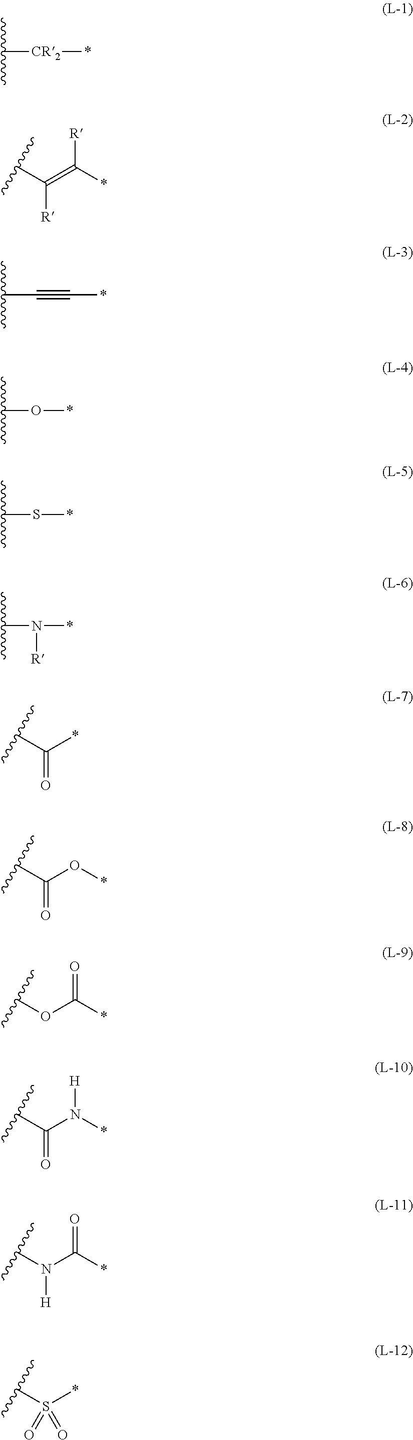

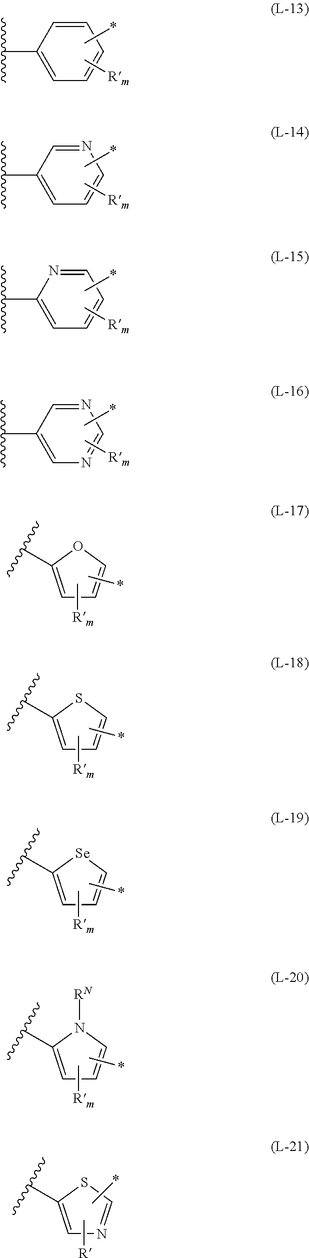

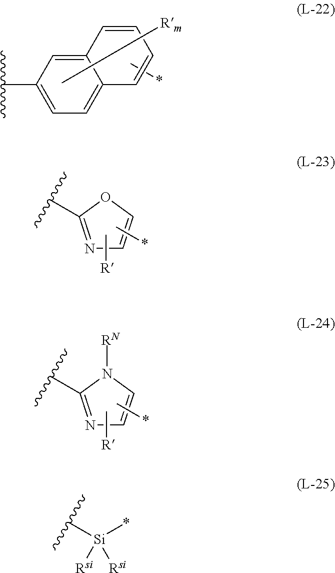

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

The compound represented by Formula (1) having the structure described above exhibits high solubility in an organic solvent. By containing the compound represented by Formula (1) in a semiconductor active layer, the organic transistor of the present invention has high carrier mobility.

Herein, EP2251342A1 (or JP2009-190999A) mainly focuses on a polymer having the skeleton of a benzobisbenzothiophene derivative or the like as a constitutional unit. However, EP2251342A1 does not have descriptions or examples showing the specific structure of the low-molecular weight compound. Furthermore, in Claim 1 of EP2251342A1, a compound is described which essentially has at least one substituent having a specific structure (an alkyl group, an alkoxy group, an alkylthio group, an alkylamino group, an alkoxycarbonyl group in which an alkyl moiety has 3 or more carbon atoms, an aryl group which may have a substituent, or a monovalent heterocyclic group or cyano group which may have a substituent) in a short axis direction of the molecule (R¹¹ and R¹² in Formula (1) of EP2251342A1). However, in such a compound, the intermolecular distance increases when the compound is crystallized, and thus overlapping of HOMO does not sufficiently occur between molecules. In addition, although Example 4 of EP2251342A1 describes the use of such a compound in an organic transistor, it does not disclose the results obtained by actually measuring carrier mobility. According to the investigation conducted by the inventors of the present invention, with the low-molecular weight compound described in EP2251342A1, it is difficult to obtain sufficient transistor characteristics, that is, high carrier mobility.

EP2301926A1 mainly focuses on a polycyclic condensed compound for an organic EL element, and does not describe a case where the compound described in the examples of EP2301926A1 is used in an organic transistor. Even if the compound is used, because the compound is an OLED material supposed to be subjected to a vapor deposition process, the solubility thereof in a general organic solvent would be low. In addition, according to the investigation conducted by the inventors of the present invention, carrier mobility of the compound is low.

In contrast, regarding the compound represented by Formula (1), by investigating various types of substituents for a condensed cyclic skeleton, that is, a mother nucleus such that overlapping of HOMO sufficiently occurs between molecules at the time of crystallization, substituting the compound with at least one of the groups represented by Formula (W) in particular, and limiting the substituents to specific substituents in a case where the substituents (R⁵ and R⁶ Formula (1) of the present invention) are present in a short axis direction of the molecule, the compound results in high carrier mobility when being used in a semiconductor active layer of an organic transistor and exhibits high solubility in an organic solvent. In order to improve the solubility in a general organic solvent, it is effective to introduce a group represented by Formula (W) into the compound, and in this way, the accomplishment of both of high mobility and high solubility that has been difficult so far can be realized.

It is preferable that the organic transistor of the present invention using the compound represented by Formula (1) shows only a slight threshold voltage shift after repeated driving. In order to reduce the threshold voltage shift after repeated driving, HOMO of the organic semiconductor material needs not to be too shallow or too deep. Furthermore, the chemical stability (particularly, resistance against air oxidation and stability against oxidation and reduction) of the organic semiconductor material, the heat stability of the film state, the high film density which makes it difficult for air or moisture to permeate the film, the film quality by which the film has small defectiveness such that charge accumulation does not easily occur, and the like are required. It is considered that because the compound represented by Formula (1) satisfies the aforementioned requirements, the organic transistor shows only a slight threshold voltage shift after repeated driving. That is, in the organic transistor showing only a slight threshold voltage shift after repeated driving, the semiconductor active layer has high chemical stability, high film density, and the like, and thus the organic transistor can effectively function as a transistor over a long period of time.

Hereinafter, preferred aspects of the compound of the present invention, the organic transistor of the present invention, and the like will be described.

<Compound Represented by Formula (1)>

The organic transistor of the present invention contains the compound represented by Formula (1) in a semiconductor active layer which will be described later. Among compounds represented by Formula (1), a compound represented by Formula (2-1) which will be described later and a compound represented by Formula (2-2) which will be described later are novel compounds. The compound represented by Formula (2-1) and the compound represented by Formula (2-2) are collectively called the compound of the present invention. The compound represented by Formula (1), particularly, the compound of the present invention is contained in a semiconductor active layer, which will be described later, in the organic transistor of the present invention. That is, the compound represented by Formula (1), particularly, the compound of the present invention can be used as a material for an organic transistor.

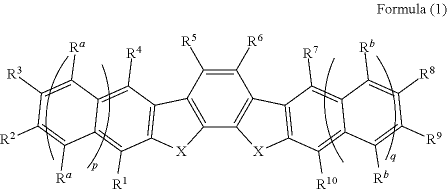

In Formula (1), each X independently represents an O atom, a S atom, or a Se atom. each X is preferably independently an O atom or a S atom. From the viewpoint of improving the carrier mobility, it is more preferable that at least one of X's is a S atom. X's are preferably the same linking group. It is particularly preferable that all of X's are a S atom.

In Formula (1), each of p and q independently represents an integer of 0 to 2. From the viewpoint of accomplishing both of mobility and solubility, it is preferable that each of p and q is 0 or 1. It is more preferable that p and q satisfy p=q=0 or p=q=1.

In Formula (1), each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, or R^{b} is a substituent represented by Formula (W), and in a case where at least one of R⁵ or R⁶ is a substituent represented by Formula (W), L in Formula (w) represented by R⁵ and R⁶ is a divalent linking group represented by the following Formula (L-2) or (L-3);

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

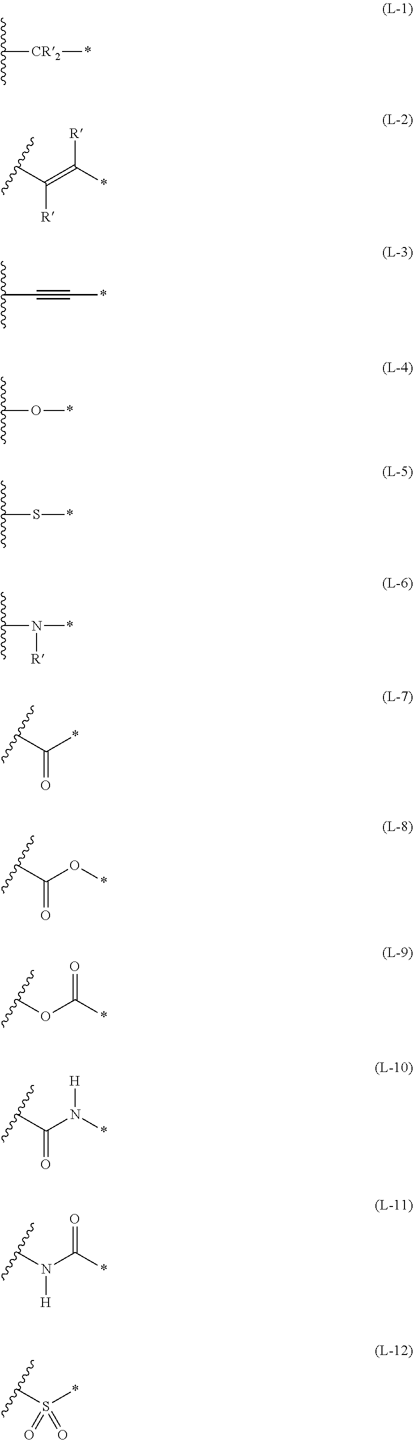

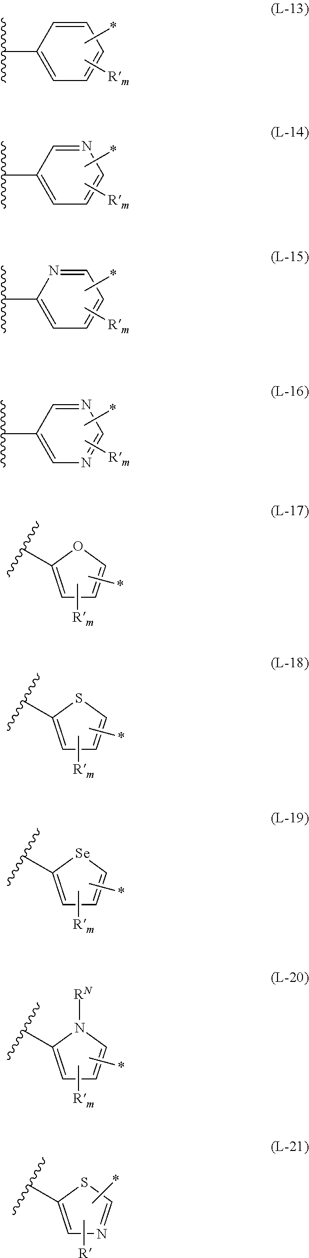

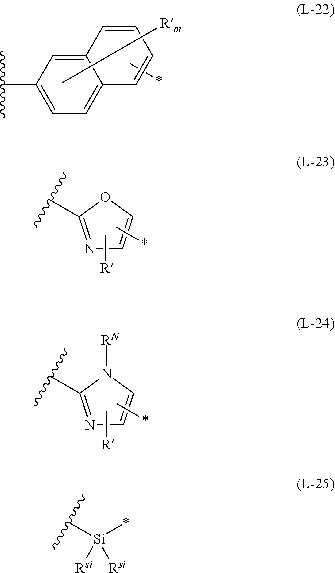

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

In Formula (1), each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, and R^{b} represents a group represented by Formula (W), and in a case where at least one of R⁵ or R⁶ is a substituent represented by Formula (W), L in Formula (W) represented by R⁵ and R⁶ is a divalent linking group represented by the following Formula (L-2) or (L-3).

The case where at least one of R⁵ or R⁶ is a substituent represented by Formula (W) corresponds to a case where none of R⁵ and R⁶ are a hydrogen atom or a halogen atom.

In a case where at least one of R⁵ or R⁶ is a substituent represented by Formula (W), L in Formula (W) represented by R⁵ and R⁶ is preferably a divalent linking group represented by the following Formula (L-3).

In a case where at least one of R⁵ or R⁶ is a substituent represented by Formula (W), both of R⁵ and R⁶ are preferably a substituent represented by Formula (W).

In a case where both of R⁵ and R⁶ are a hydrogen atom or a halogen atom, each of R¹ to R⁴, R⁷ to R¹⁰, R^{a}, and R^{b} is a hydrogen atom, a halogen atom, or a substituent represented by Formula (W), and at least one or more of R¹, R², R³, R⁴, R⁷, R⁸, R⁹, R¹⁰, R^{a}, or R^{b} are a substituent represented by Formula (W).

In Formula (1), examples of the halogen atom represented by R¹ to R¹⁰, R^{a}, and R^{b} include a fluorine atom, a chlorine atom, a bromine atom, and an iodine atom. The halogen atom is preferably a fluorine atom, a chlorine atom, or a bromine atom, more preferably a fluorine atom or a chlorine atom, and particularly preferably a fluorine atom.

In the compound represented by Formula (1), the number of halogen atoms represented by R¹ to R¹⁰, R^{a}, and R^{b} is preferably 0 to 4, more preferably 0 to 2, particularly preferably 0 or 1, and more particularly preferably 0.

The group represented by Formula (W) that R¹ to R¹⁰, R^{a}, and R^{b} in Formula (1) represent will be described.

-L-R Formula (W)

in Formula (W), L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group:

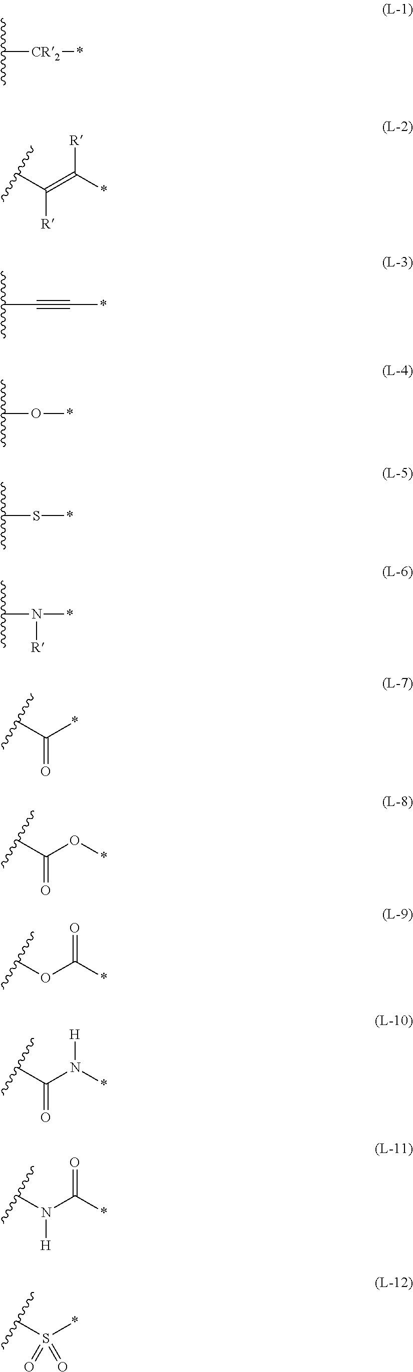

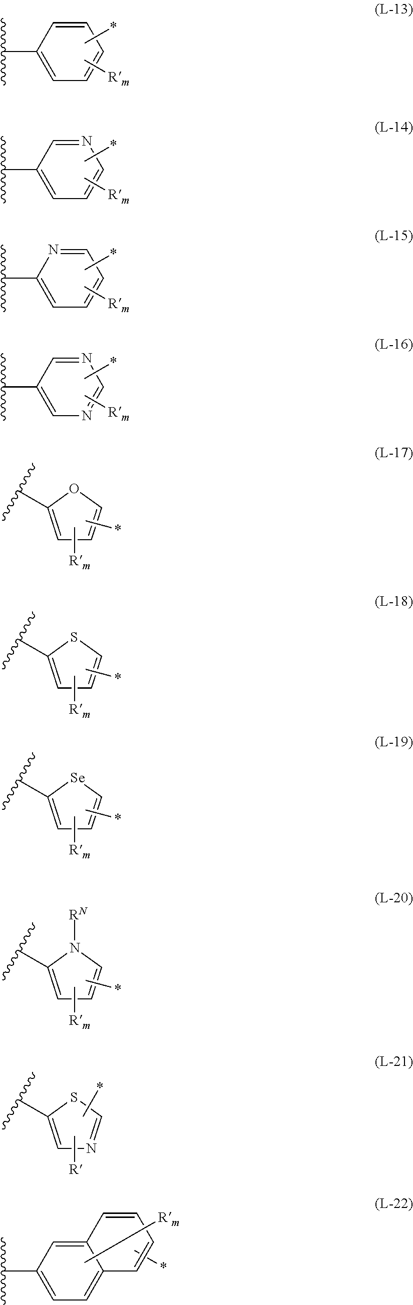

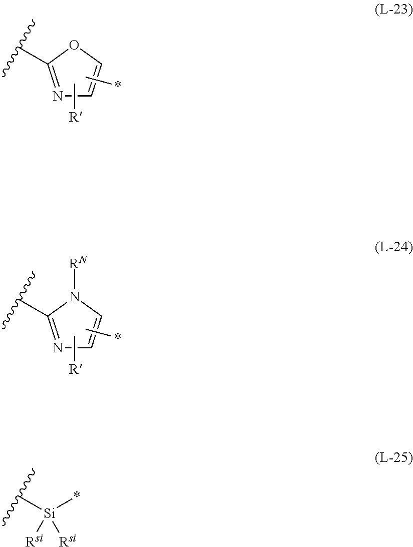

in Formulae (L-1) to (L-25), the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

In Formula (W), L is a divalent linking group represented by any one of the following Formulae (L-1) to (L-25) or a divalent linking group in which two or more divalent linking groups represented by any of the following Formulae (L-1) to L-25) are bonded to each other.

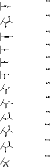

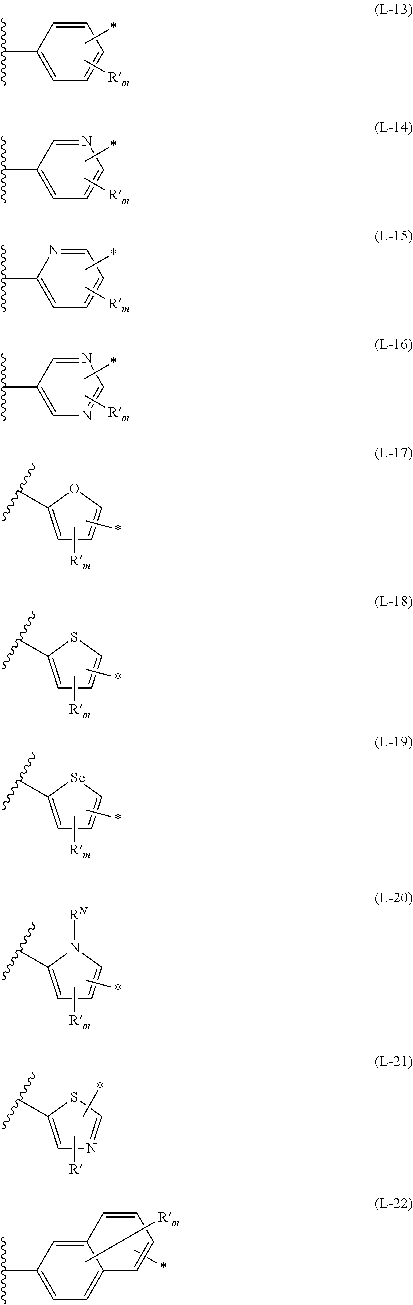

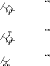

In Formulae (L-1) to (L-25), the portion of a wavy line represents a position of bonding to a condensed cyclic skeleton. Here, in a case where L represents a divalent linking group in which two or more divalent linking groups represented by any one of Formulae (L-1) to (L-25) are bonded to each other, the portion of a wavy line represents a position of bonding to * of a divalent linking group represented by any of Formulae (L-1) to (L-25).

In Formulae (L-1) to (L-25), * represents a position of bonding to any one of the portion of a wavy line and R in a divalent linking group represented by any of Formulae (L-1) to (L-25).

m in Formula (L-13) represents 4, m in Formulae (L-14) and (L-15) represents 3, m in Formulae (L-16) to (L-20) represents 2, and m in Formula (L-22) represents 6.

Each R′ in Formulae (L-1), (L-2), (L-6), and (L-13) to (L-24) independently represents a hydrogen atom or a substituent.

In Formulae (L-1) to (L-25), R^{N} represents a hydrogen atom or a substituent.

In Formulae (L-1) to (L-25), each R^{si} independently represents a hydrogen atom, an alkyl group, an alkenyl group, or an alkynyl group.

In Formulae (L-1) to (L-2), each R′ may form a condensed ring by being bonded to R adjacent to L.

A divalent linking group represented by any of Formulae (L-17) to (L-21), (L-23), and (L-24) is more preferably a divalent linking group represented by any of the following Formulae (L-17A) to (L-21A), (L-23A), and (L-24A).

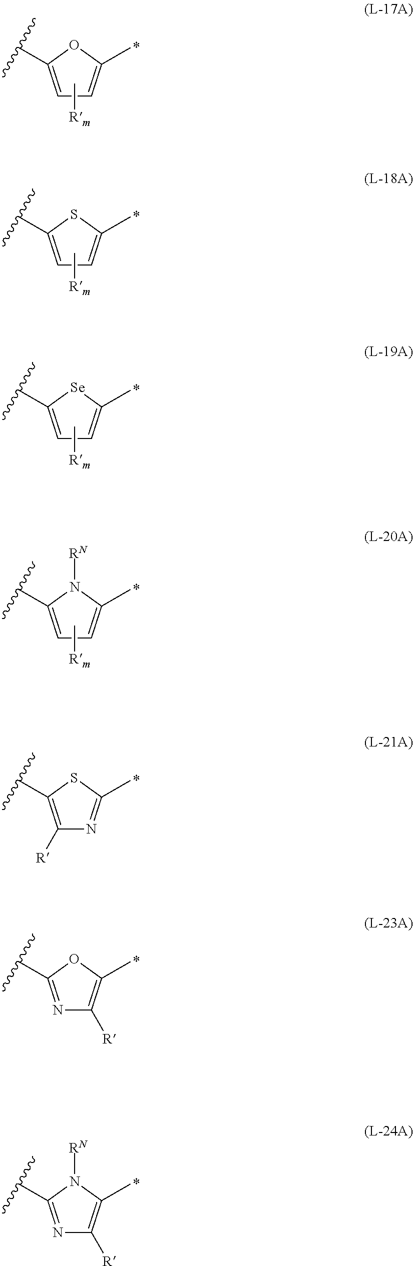

In a case where a substituted or unsubstituted alkyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group is present on the terminal of the substituent represented by Formula (W), the substituent can be interpreted as either a substituent consisting of only —R in Formula (W) or a substituent consisting of —R-L in Formula (W).

In the present invention, in a case where a substituted or unsubstituted alkyl group whose main chain consists of N carbon atoms is present on the terminal of the substituent represented by Formula (W), the substituent is interpreted as -L-R in Formula (W) by including as much linking groups represented by —CR^{R}₂— as possible in R from the terminal of the substituent represented by Formula (W), and is not interpreted as a substituent composed solely of —R. Herein, R^{R} represents a hydrogen atom or a substituent. Specifically, the substituent is interpreted as a substituent in which “one (L-1) corresponding to L in Formula (W)” is bonded to “a substituted or unsubstituted alkyl group corresponding to R in Formula (W) having a main chain consisting of (N−1) carbon atoms”. For example, in a case where a n-octyl group as an alkyl group having 8 carbon atoms is present on the terminal of the substituent, the substituent is interpreted as a substituent in which one (L-1), wherein two R's are hydrogen atoms, is boned to a n-heptyl group having 7 carbon atoms. In a case where the substituent represented by Formula (W) is an alkyl group, for example, how to make a differentiation between L and R is as follows, and L represents a divalent linking group represented by Formula (L-1).

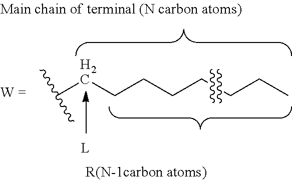

In a case where the substituent represented by Formula (W) is an alkoxy group having 8 carbon atoms, the alkoxy group is interpreted as a substituent in which one linking group represented by Formula (L-4) that is —O—, one linking group represented by Formula (L-1) in which two R's are hydrogen atoms, and a n-heptyl group having 7 carbon atoms are bonded to each other. In a case where the substituent represented by Formula (W) is a group other than an alkyl group, how to make a differentiation between L and R is as follows, and L represents a divalent linking group in which “a divalent linking group other than the linking group represented by Formula (L-1)” and “a divalent linking group represented by Formula (L-1)” are bonded to each other.

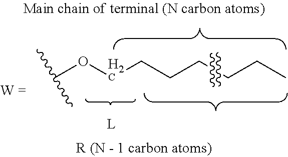

In contrast, in the present invention, in a case where an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group is present on the terminal of the substituent represented by Formula (W), the substituent is interpreted as a substituent consisting solely of R in Formula (W) by including as much linking groups as possible in R from the terminal of the substituent represented by Formula (W), but is not interpreted as -L-R. For example, in a case where a —(OCH₂CH₂)—(OCH₂CH₂)—OCH₃ group is present on the terminal of the substituent, the substituent is interpreted as a substituent consisting of only an oligo-oxyethylene group in which the repetition number v of an oxyethylene unit is 2. In a case where an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group is present on the terminal of the substituent represented by Formula (W), how to make a differentiation between L and R is as follows, for example.

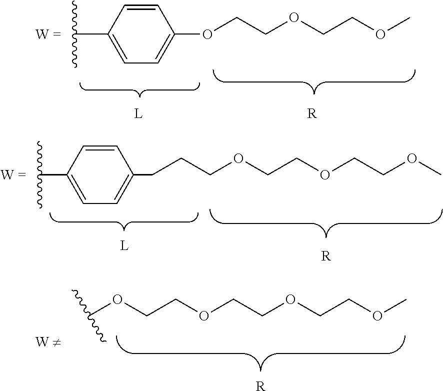

In a case where L forms a linking group in which divalent linking groups represented by any one of Formulae (L-1) to (L-25) are bonded to each other, the number of the divalent linking groups bonded to each other represented by any one of Formulae (L-1) to (L-25) is preferably 2 to 4, and more preferably 2 or 3.

Examples of the substituent R′ in Formulae (L-1), (L-2), (L-6), and (L-13) to (L-24) include a halogen atom, an alkyl group (including an alkyl group having 1 to 40 carbon atoms such as a methyl group, an ethyl group, a propyl group, a butyl group, a pentyl group, a hexyl group, a heptyl group, an octyl group, a nonyl group, a decyl group, an undecyl group, a dodecyl group, a tridecyl group, a tetradecyl group, or a pentadecyl group; here, the alkyl group also includes a 2,6-dimethyloctyl group, a 2-decyltetradecyl group, a 2-hexyldodecyl group, a 2-ethyloctyl group, a 2-decyltetradecyl group, a 2-butyldecyl group, a 1-octylnonyl group, a 2-ethyloctyl group, a 2-octyltetradecyl group, a 2-ethylhexyl group, a cycloalkyl group, a bicycloalkyl group, a tricycloalkyl group, and the like), an alkenyl group (including a 1-pentenyl group, a cycloalkenyl group, a bicycloalkenyl group, and the like), an alkynyl group (including a 1-pentynyl group, a trimethylsilylethynyl group, a triethylsilylethynyl group, a tri-i-propylsilylethynyl group, a 2-p-propylphenylethynyl group, and the like), an aryl group (including an aryl group having 6 to 20 carbon atoms such as a phenyl group, a naphthyl group, a p-pentylphenyl group, a 3,4-dipentylphenyl group, a p-heptoxyphenyl group, a 3,4-diheptoxyphenyl group, and the like), a hetero ring group (may be referred to as a heterocyclic group as well, including a 2-hexylfuranyl group and the like), a cyano group, a hydroxyl group, a nitro group, an acyl group (including a hexanoyl group, a benzoyl group, and the like), an alkoxy group (including a butoxy group and the like), an aryloxy group, a silyloxy group, a heterocyclic oxy group, an acyloxy group, a carbamoyloxy group, an amino group (including an anilino group), an acylamino group, an aminocarbonylamino group (including a ureide group), alkoxy- and aryloxycarbonylamino groups, alkyl- and aryl sulfonylamino groups, a mercapto group, alkyl- and arylthio groups (including a methylthio group, an octylthio group, and the like), a heterocyclic thio group, a sulfamoyl group, a sulfo group, alkyl- and aryl sulfinyl groups, alkyl- and aryl sulfonyl groups, alkyloxy- and aryloxycarbonyl groups, a carbamoyl group, aryl- and heterocyclic azo group, an imide group, a phosphino group, a phosphinyl group, a phosphinyloxy group, a phosphinylamino group, a phosphono group, a silyl group (a ditrimethylsiloxy methylbutoxy group), a hydrazino group, a ureide group, a boronic acid group (—B(OH)₂), a phosphate group (—OPO(OH)₂), a sulfate group (—OSO₃H), and other known substituents.

These substituents may further have the above substituents. In addition, in a case where the compound represented by Formula (1) is a polymer compound having a repeating structure, each of R¹ to R⁸ may have a group derived from a polymerizable group.

Among these, the substituent R′ in Formula (L-6) is preferably an alkyl group. In a case where R′ in (L-6) is an alkyl group, the number of carbon atoms of the alkyl group is preferably 1 to 9, more preferably 4 to 9 from the viewpoint of the chemical stability and the carrier transport properties, and even more preferably 5 to 9. In a case where R′ in (L-6) is an alkyl group, the alkyl group is preferably a linear alkyl group because then the carrier mobility can be improved.

R^{N} represents a hydrogen atom or a substituent, and examples of R^{N} include those exemplified above as substituents that can be adopted as R described above. Among these, a hydrogen atom or a methyl group is preferable as R^{N}.

Each R^{si} independently represents a hydrogen atom, an alkyl group, an alkenyl group, or an alkynyl group, and is preferably an alkyl group. The alkyl group that can be adopted as R^{si} is not particularly limited, and the preferred range of the alkyl group that can be adopted as R^{si} is the same as the preferred range of an alkyl group that can be adopted as a silyl group in a case where R is a silyl group. The alkenyl group that can be adopted as R^{si} is not particularly limited. However, the alkenyl group is preferably a substituted or unsubstituted alkenyl group and more preferably a branched alkenyl group, and the number of carbon atoms of the alkenyl group is preferably 2 or 3. The alkynyl group that can be adopted as R^{si} is not particularly limited. However, the alkynyl group is preferably a substituted or unsubstituted alkynyl group and more preferably a branched alkynyl group, and the number of carbon atoms of the alkynyl group is preferably 2 or 3.

In a case where L includes divalent linking groups represented by Formulae (L-13) to (L-25), any one of the divalent linking groups represented by Formulae (L-13) to (L-25) is preferably bonded to R via the divalent linking group represented by Formula (L-1).

L is preferably a divalent linking group represented by any one of Formulae (L-1) to (L-5), (L-13), (L-17), and (L-18) or a divalent linking group in which two or more divalent linking groups represented by any one of Formulae (L-1) to (L-5), (L-13), (L-17), and (L-18) are bonded to each other, more preferably a divalent linking group represented by any one of Formulae (L-1) to (L-4) or a divalent linking group in which a divalent linking group represented by any one of Formulae (L-2) to (L-4) is bonded to a divalent linking group represented by Formula (L-1), and particularly preferably a divalent linking group represented by Formula (L-1). It is preferable that the divalent linking group represented by Formula (L-1) is bonded to the R side of a divalent linking group in which the divalent linking group represented by any one of Formulae (L-2) to (L-4) is bonded to the divalent linking group represented by Formula (L-1).

From the viewpoint of the chemical stability and the carrier transport properties, L is particularly preferably a divalent linking group including the divalent linking group represented by Formula (L-1), more particularly preferably a divalent linking group represented by Formula (L-1).

In Formula (W), R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted silyl group.

In Formula (W), in a case where L adjacent to R is a divalent linking group represented by Formula (L-1), R is preferably a substituted or unsubstituted alkyl group, an oxyethylene group, an oligo-oxyethylene group in which a repetition number v of an oxyethylene unit is equal to or greater than 2, a siloxane group, or an oligosiloxane group having two or more silicon atoms, and more preferably a substituted or unsubstituted alkyl group.

In Formula (W), in a case where L adjacent to R is a divalent linking group represented by any of Formulae (L-2) and (L-4) to (L-25), R is more preferably a substituted or unsubstituted alkyl group.

In Formula (W), in a case where L adjacent to R is a divalent linking group represented by Formula (L-3), R is preferably a substituted or unsubstituted alkyl group or a substituted or unsubstituted silyl group.

In a case where R is a substituted or unsubstituted alkyl group, the number of carbon atoms thereof is preferably 4 to 18, more preferably 6 to 15 from the viewpoint of the chemical stability and the carrier transport properties, and even more preferably 6 to 12. If R is a long-chain alkyl group within the above range, the alkyl group is particularly preferably a long-chain linear alkyl group because then the linearity of the molecule is improved, and hence the carrier mobility can be improved.

In a case where R represents an alkyl group, the alkyl group may be a linear, branched, or cyclic alkyl group. However, the alkyl group is preferably a linear alkyl group because then the linearity of the molecule is improved, and hence the carrier mobility can be improved.

In contrast, from the viewpoint of improving the solubility in an organic solvent, R is preferably a branched alkyl group.

In a case where R is an alkyl group having a substituent, examples of the substituent include a halogen atom and the like (at this time, R becomes a haloalkyl group), and the halogen atom is preferably a fluorine atom. In a case where R is an alkyl group having a fluorine atom, a perfluoroalkyl group may be formed by the substitution of all of the hydrogen atoms with the fluorine atom. Here, R is preferably an unsubstituted alkyl group.

In the present specification, in a case where the R is an ethyleneoxy group or an oligo-oxyethylene group in which a repetition number v of an oxyethylene unit is equal to or greater than 2, the “oligo-oxyethylene group” represented by R refers to a group represented by —(OCH₂CH₂)ᵥOY (the repetition number v of an oxyethylene unit represents an integer of equal to or greater than 2, and Y on the terminal represents a hydrogen atom or a substituent). In a case where Y on the terminal of the oligo-oxyethylene group is a hydrogen atom, the terminal becomes a hydroxy group. The repetition number v of the oxyethylene unit is preferably 2 to 4, and more preferably 2 or 3. It is preferable that the hydroxy group on the terminal of the oligo-oxyethylene group is sealed. That is, it is preferable that Y represents a substituent. In this case, the hydroxy group is preferably sealed with an alkyl group having 1 to 3 carbon atoms. In other words, Y is preferably an alkyl group having 1 to 3 carbon atoms, more preferably a methyl group or an ethyl group, and particularly preferably a methyl group.

In a case where R is a siloxane group or an oligosiloxane group having two or more silicon atoms, the repetition number of the siloxane unit is preferably 2 to 4, and more preferably 2 or 3. Furthermore, it is preferable that hydrogen atoms or alkyl groups are boned to the Si atoms. In a case where alkyl groups are boned to the Si atoms, the number of carbon atoms of each alkyl group is preferably 1 to 3. For example, it is preferable that methyl groups or ethyl groups are bonded to the Si atoms. The same alkyl groups may be bonded to the Si atoms, or different alkyl groups or hydrogen atoms may be bonded to the Si atoms. In addition, all of the siloxane units constituting the oligosiloxane group may be the same as or different from each other, but it is preferable that they are the same as each other.

In a case where L adjacent to R is a divalent linking group represented by Formula (L-3), R is preferably a substituted or unsubstituted silyl group. In a case where R is a substituted or unsubstituted silyl group, a substituted silyl group is preferable as R. The substituent of the silyl group is not particularly limited. However, the substituent is preferably a substituted or unsubstituted alkyl group, and more preferably a branched alkyl group. In a case where R is a trialkylsilyl group, the number of carbon atoms of each alkyl group bonded to the Si atoms is preferably 1 to 3. For example, it is preferable that methyl groups, ethyl groups, or isopropyl groups are bonded to the Si atoms. The same alkyl groups or different alkyl groups may be bonded to the Si atoms. In a case where R is a trialkylsilyl group further having a substituent on the alkyl group, the substituent is not particularly limited.

The total number of carbon atoms contained in the group represented by Formula (W), that is, the total number of carbon atoms contained in L and R is not particularly limited and can be 2 to 20, for example. However, the total number of carbon atoms is preferably 4 to 18. If the total number of carbon atoms contained in L and R is equal to or greater than the lower limit of the above range, the carrier mobility is improved, and the solubility is improved. If the total number of carbon atoms contained in L and R is equal to or less than the upper limit of the above range, the driving voltage is lowered.

The total number of carbon atoms contained in L and R is preferably 4 to 24, more preferably 6 to 21, and particularly preferably 6 to 18.

In the present invention, in a case where p=q=0 in Formula (1), the total number of carbon atoms contained in the group represented by Formula (W) is preferably 4 to 24, more preferably 6 to 21, and particularly preferably 6 to 18.

Particularly, in a case where p=q=0 in Formula (1), and the substituent represented by Formula (W) is an alkyl group, the substituent represented by Formula (W) is preferably a linear alkyl group having 4 to 18 carbon atoms or a branched alkyl group having 4 or more carbon atoms, more preferably a linear alkyl group having 4 to 14 carbon atoms or a branched alkyl group having 4 to 14 carbon atoms, particularly preferably a linear alkyl group having 5 to 14 carbon atoms or a branched alkyl group having 5 to 14 carbon atoms, more particularly preferably a linear alkyl group having 5 to 10 carbon atoms or a branched alkyl group having 6 to 12 carbon atoms, and even more particularly preferably a linear alkyl group having 6 to 10 obtained or a branched alkyl group having 8 to 12 carbon atoms.

In the present invention, in a case where p=q=1 in Formula (1), the total number of carbon atoms contained in the group represented by Formula (W) is preferably 4 to 24, more preferably 4 to 21, particularly preferably 5 to 18, more particularly preferably 6 to 18, and even more particularly preferably 6 to 16.

Particularly, in a case where p=q=1 in Formula (1), and the substituent represented by Formula (W) is an alkyl group, the substituent represented by Formula (W) is not particularly limited. However, the substituent is preferably a linear alkyl group having 4 to 18 carbon atoms or a branched alkyl group having 4 to 18 carbon atoms, more preferably a linear alkyl group having 4 to 14 carbon atoms or a branched alkyl group having 4 to 14 carbon atoms, particularly preferably a linear alkyl group having 5 to 12 carbon atoms or a branched alkyl group having 5 to 12 carbon atoms, more particularly preferably a linear alkyl group having 6 to 12 carbon atoms or a branched alkyl group having 6 to 12 carbon atoms, and even more particularly preferably a linear alkyl group having 6 to 10 carbon atoms or a branched alkyl group having 6 to 10 carbon atoms.

In a case where L includes divalent linking groups represented by Formulae (L-13) to (L-25), R and L in Formula (W) are preferably combined such that R is an alkyl group, and L is any one of the divalent linking groups represented by Formulae (L-13) to (L-25) and bonded to R via a divalent linking group represented by Formula (L-1).

Especially, R and L in Formula (W) are preferably combined such that L is a divalent linking group represented by any one of Formulae (L-1) to (L-5), (L-13), (L-17), and (L-18) or a divalent linking group in which two or more divalent linking groups represented by any one of Formulae (L-1) to (L-5), (L-13), (L-17), and (L-18) are bonded to each other, and R is a substituted or unsubstituted alkyl group; more preferably combined such that L is a divalent linking group represented by any one of Formulae (L-1) to (L-4) or a divalent linking group in which a divalent linking group represented by any one of Formulae (L-2) to (L-4) is bonded to a divalent linking group represented by Formula (L-1), and R is a substituted or unsubstituted alkyl group; and particularly preferably combined such that L is a divalent linking group represented by Formula (L-1), and R is a substituted or unsubstituted alkyl group.

In the compound represented by Formula (1), the number of groups adopted as one of R¹ to R¹⁰, R^{a}, and R^{b} and represented by Formula (W) is preferably 1 to 4 from the viewpoint of improving the carrier mobility and the solubility in an organic solvent, more preferably 1 or 2, and particularly preferably 2.

The group represented by Formula (W) is positioned in any of R¹ to R¹⁰, R^{a}, and R^{b} without particular limitation. In the present invention, from the viewpoint of improving the carrier mobility and improving the solubility in an organic solvent, it is preferable that in Formula (1), each of R¹, R⁴ to R⁷, R¹⁰, R^{a}, and R^{b} is independently a hydrogen atom or a halogen atom, each of R², R³, R⁸, and R⁹ is independently a hydrogen atom, a halogen atom, or a substituent represented by Formula (W), and at least one of R², R³, R⁸, or R⁹ is a substituent represented by Formula (W).

In the present invention, it is more preferable that each of R¹, R³ to R⁸, and R¹⁰ independently represents a hydrogen atom or a halogen atom, each of R² and R⁹ is independently a hydrogen atom, a halogen atom, or a substituent represented by the following Formula (W), and at least one of R² or R⁹ is a substituent represented by Formula (W).

In the present invention, it is particularly preferable that both of R² and R⁹ are a substituent represented by Formula (W), and both of R³ and R⁸ are a hydrogen atom or a halogen atom. Alternatively, it is particularly preferable that both of R³ and R⁸ are a substituent represented by Formula (W), and both of R² and R⁹ are a hydrogen atom or a halogen atom.

In the present invention, it is more particularly preferable that both of R² and R⁹ are a substituent represented by Formula (W), and both of R³ and R⁸ are a hydrogen atom or a halogen atom. Alternatively, it is more particularly preferable that both of R³ and R⁸ are a substituent represented by Formula (W), and both of R² and R⁹ are a hydrogen atom or a halogen atom.

In Formula (1), two or more groups represented by R¹ to R¹⁰, R^{a}, and R^{b} may or may not form a ring by being bonded to each other. However, it is preferable that they do not form a ring by being bonded to each other.

The compound represented by Formula (1) is preferably a compound represented by the following Formula (2-1) or (2-2), and particularly preferably a compound represented by Formula (2-1) from the viewpoint of accomplishing both the high carrier mobility and the high solubility.

First, a case where the compound represented by Formula (1) is a compound represented by the following Formula (2-1) will be described.

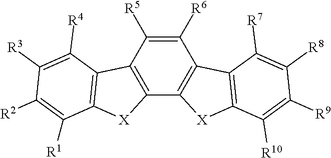

In Formula (2-1),

each of R¹, R³ to R⁸, and R¹⁰ independently represents a hydrogen atom or a halogen atom,

each of R² and R⁹ is independently a hydrogen atom, a halogen atom, or a substituent represented by the following Formula (W), at least one of R² or R⁹ is a substituent represented by Formula (W), and in a case where the substituent represented by Formula (W) is an alkyl group, the substituent represented by Formula (W) is limited to a linear alkyl group having 4 to 18 carbon atoms or a branched alkyl group having 4 or more carbon atoms.

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

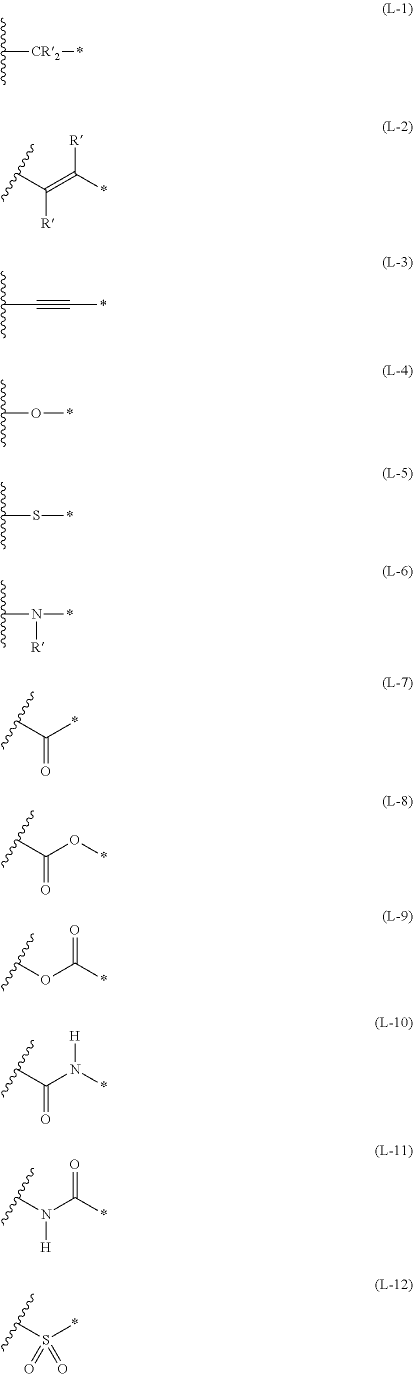

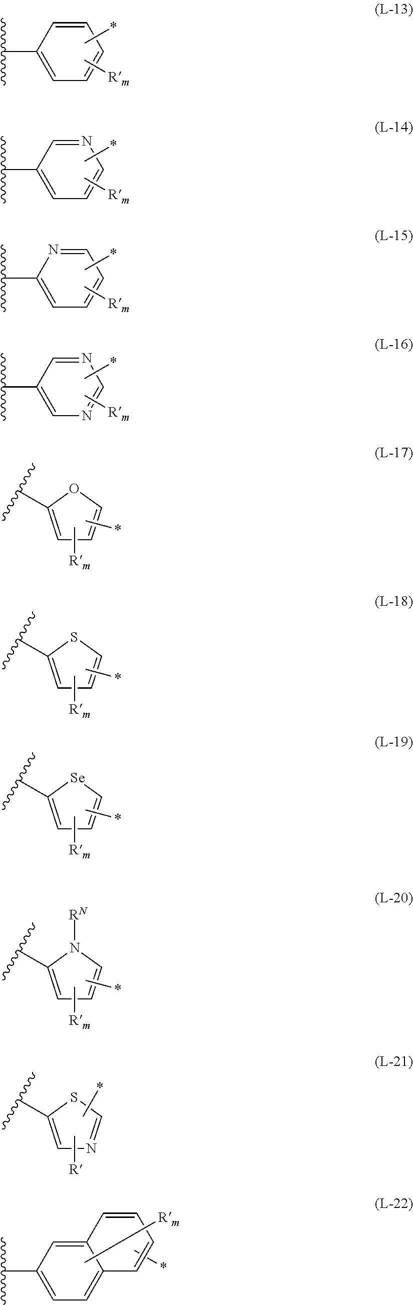

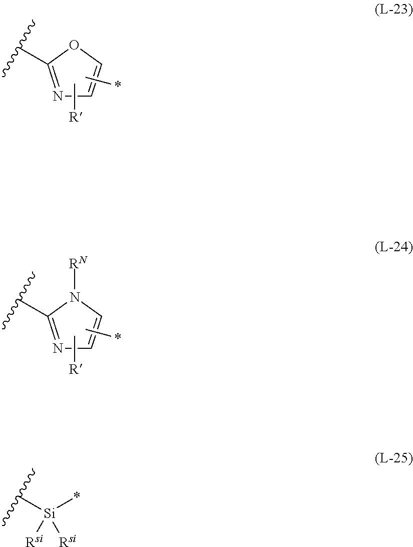

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

In Formula (2-1), each X independently represents an O atom, a S atom, or a Se atom.

The preferred range of X in Formula (2-1) is the same as the preferred range of X in Formula (1).

In Formula (2-1), each of R¹, R³ to R⁸, and R¹⁰ independently represents a hydrogen atom or a halogen atom. The preferred range of R¹, R³ to R⁸, and R¹⁰ in Formula (2-1) is the same as the preferred range of R¹, R³ to R⁸, and R¹⁰ in Formula (1).

In Formula (2-1), each of R² and R⁹ is independently s hydrogen atom, a halogen atom, or a substituent represented by Formula (W), and at least one of R² or R⁹ is a substituent represented by Formula (W). Here, in a case where the substituent represented by Formula (W) is an alkyl group, the substituent represented by Formula (W) is limited to a linear alkyl group having 4 to 18 carbon atoms or a branched alkyl group having 4 or more carbon atoms. The preferred range of R² and R⁹ in Formula (2-1) is the same as the preferred range of R² and R⁹ in a case where p=q=0 in Formula (1).

Next, a case where the compound represented by Formula (1) is a compound represented by the following Formula (2-2) will be described.

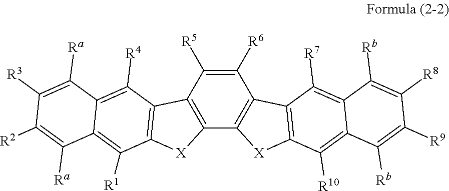

In Formula (2-2),

each X independently represents an O atom, a S atom, or a Se atom,

each of R¹, R⁴ to R⁷, R¹⁰, R^{a}, and R^{b} is independently a hydrogen atom or a halogen atom,

each of R², R³, R⁸, and R⁹ independently represents a hydrogen atom, a halogen atom, or a substituent represented by the following Formula (W), and at least one of R², R³, R⁸, or R⁹ represents a substituent represented by Formula (W).

-L-R Formula (W)

in Formula (W),

L represents a divalent linking group represented by any one of the following Formulae (L-1) to (L-25) or a divalent linking group in which two or more divalent linking groups represented by any one of the following Formulae (L-1) to (L-25) are bonded to each other, and

R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group;

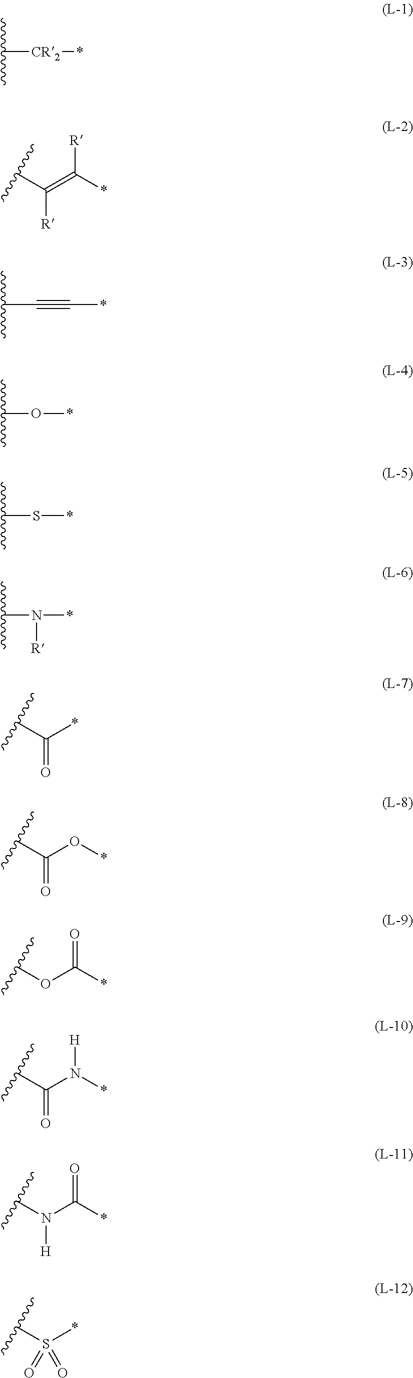

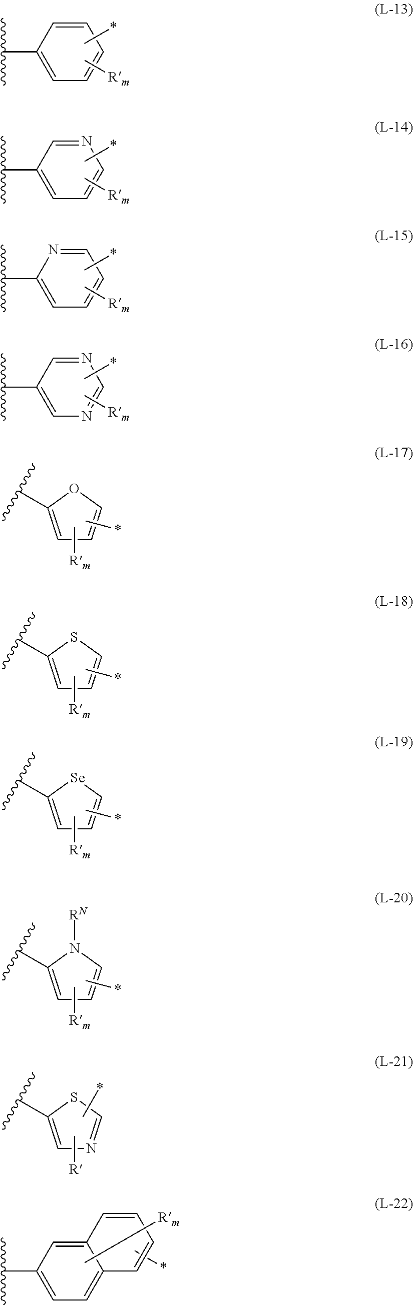

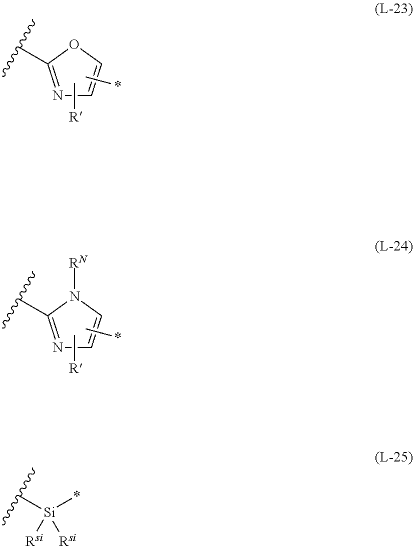

in Formulae (L-1) to (L-25),

the portion of a wavy line represents a position of bonding to a condensed cyclic skeleton,

m in Formula (L-13) represents 4, m in Formulae (L-14) and (L-15) represents 3, m in Formulae (L-16) to (L-20) represents 2, m in Formula (L-22) represents 6,

each R′ in Formulae (L-1), (L-2), (L-6), and (L-13) to (L-24) independently represents a hydrogen atom or a substituent,

R^{N} represents a hydrogen atom or a substituent, and

each R^{si} independently represents a hydrogen atom, an alkyl group, an alkenyl group, or an alkynyl group.

In Formula (2-2), each X independently represents an O atom, a S atom, or a Se atom. The preferred range of X in Formula (2-2) is the same as the preferred range of X in Formula (1).

In Formula (2-2), each of R¹, R⁴ to R⁷, R¹⁰, R^{a}, and R^{b} is independently a hydrogen atom or a halogen atom. The preferred range of R¹, R⁴ to R⁷, R¹⁰, R^{a}, and R^{b} in Formula (2-2) is the same as the preferred range of R¹, R⁴ to R⁷, R¹⁰, R^{a}, and R^{b} in Formula (1).

In Formula (2-2), each of R², R³, R⁸, and R⁹ independently represents a hydrogen atom, a halogen atom, or a substituent represented by the following Formula (W), and at least one of R², R³, R⁸, or R⁹ represents a substituent represented by Formula (W). The preferred range of R², R³, R⁸, and R⁹ in Formula (2-2) is the same as the preferred range of R², R³, R⁸, and R⁹ in a case where p=q=1 in Formula (1).

Specific examples of the compound represented by Formula (1) will be shown below. However, the compound represented by Formula (1) that can be used in the present invention is not limited to the specific examples.

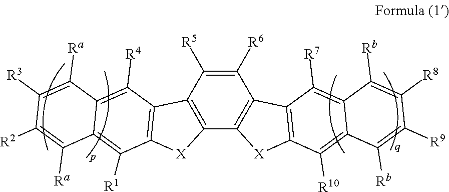

In Formula (1′), each of R^{a} and R^{b} independently represents a hydrogen atom, and each of X, p, q, and R¹ to R¹⁰ represents the following groups. In the following tables, Ph represents a phenyl group or a phenylene group.

TABLE 1

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 1 | S | 0 | 0 | H | C3H7 | H | H | H | H | H | H | C3H7 | H |
| Compound example 2 | S | 0 | 0 | H | C4H9 | H | H | H | H | H | H | C4H9 | H |
| Compound example 3 | S | 0 | 0 | H | C5H11 | H | H | H | H | H | H | C5H11 | H |
| Compound example 4 | S | 0 | 0 | H | C6H13 | H | H | H | H | H | H | C6H13 | H |
| Compound example 5 | S | 0 | 0 | H | C7H15 | H | H | H | H | H | H | C7H15 | H |
| Compound example 6 | S | 0 | 0 | H | C8H17 | H | H | H | H | H | H | C8H17 | H |
| Compound example 7 | S | 0 | 0 | H | C9H19 | H | H | H | H | H | H | C9H19 | H |
| Compound example 8 | S | 0 | 0 | H | C10H21 | H | H | H | H | H | H | C10H21 | H |
| Compound example 9 | S | 0 | 0 | H | C11H23 | H | H | H | H | H | H | C11H23 | H |
| Compound example 10 | S | 0 | 0 | H | C12H25 | H | H | H | H | H | H | C12H25 | H |
| Compound example 11 | S | 0 | 0 | H | C13H27 | H | H | H | H | H | H | C13H27 | H |
| Compound example 12 | S | 0 | 0 | H | C14H29 | H | H | H | H | H | H | C14H29 | H |
| Compound example 13 | S | 0 | 0 | H | C15H31 | H | H | H | H | H | H | C15H31 | H |
| Compound example 14 | S | 0 | 0 | H | C16H33 | H | H | H | H | H | H | C16H33 | H |
| Compound example 15 | S | 0 | 0 | H | C17H35 | H | H | H | H | H | H | C17H35 | H |
| Compound example 16 | S | 0 | 0 | H | C18H37 | H | H | H | H | H | H | C18H37 | H |
| Compound example 17 | S | 1 | 1 | H | C5H11 | H | H | H | H | H | H | C5H11 | H |
| Compound example 18 | S | 1 | 1 | H | C6H13 | H | H | H | H | H | H | C6H13 | H |
| Compound example 19 | S | 1 | 1 | H | C7H15 | H | H | H | H | H | H | C7H15 | H |
| Compound example 20 | S | 1 | 1 | H | C8H17 | H | H | H | H | H | H | C8H17 | H |
| Compound example 21 | S | 1 | 1 | H | C9H19 | H | H | H | H | H | H | C9H19 | H |
| Compound example 22 | S | 1 | 1 | H | C10H21 | H | H | H | H | H | H | C10H21 | H |
| Compound example 23 | S | 1 | 1 | H | C11H23 | H | H | H | H | H | H | C11H23 | H |
| Compound example 24 | S | 1 | 1 | H | C12H25 | H | H | H | H | H | H | C12H25 | H |
| Compound example 25 | S | 1 | 1 | H | C13H27 | H | H | H | H | H | H | C13H27 | H |
| Compound example 26 | S | 1 | 1 | H | C14H29 | H | H | H | H | H | H | C14H29 | H |
| Compound example 27 | S | 1 | 1 | H | C15H31 | H | H | H | H | H | H | C15H31 | H |
| Compound example 28 | S | 1 | 1 | H | C16H33 | H | H | H | H | H | H | C16H33 | H |
| Compound example 29 | S | 1 | 1 | H | C17H35 | H | H | H | H | H | H | C17H35 | H |
| Compound example 30 | S | 1 | 1 | H | C18H37 | H | H | H | H | H | H | C18H37 | H |

TABLE 2

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 31 | S | 0 | 0 | H | p-C5H11—Ph—* | H | H | H | H | H | H | p-C5H11—Ph—* | H |
| Compound example 32 | S | 0 | 0 | H | p-C6H13—Ph—* | H | H | H | H | H | H | p-C6H13—Ph—* | H |
| Compound example 33 | S | 0 | 0 | H | p-C7H15—Ph—* | H | H | H | H | H | H | p-C7H15—Ph—* | H |
| Compound example 34 | S | 0 | 0 | H | p-C8H17—Ph—* | H | H | H | H | H | H | p-C8H17—Ph—* | H |
| Compound example 35 | S | 0 | 0 | H | p-C9H19—Ph—* | H | H | H | H | H | H | p-C9H19—Ph—* | H |
| Compound example 36 | S | 0 | 0 | H | p-C10H21—Ph—* | H | H | H | H | H | H | p-C10H21—Ph—* | H |
| Compound example 37 | S | 0 | 0 | H | p-C11H23—Ph—* | H | H | H | H | H | H | p-C11H23—Ph—* | H |
| Compound example 38 | S | 0 | 0 | H | p-C12H25—Ph—* | H | H | H | H | H | H | p-C12H25—Ph—* | H |
| Compound example 39 | S | 0 | 0 | H | p-C13H27—Ph—* | H | H | H | H | H | H | p-C13H27—Ph—* | H |
| Compound example 40 | S | 0 | 0 | H | p-C14H29—Ph—* | H | H | H | H | H | H | p-C14H29—Ph—* | H |
| Compound example 41 | S | 0 | 0 | H | p-C15H31—Ph—* | H | H | H | H | H | H | p-C15H31—Ph—* | H |
| Compound example 42 | S | 0 | 0 | H | p-C16H33—Ph—* | H | H | H | H | H | H | p-C16H33—Ph—* | H |
| Compound example 43 | S | 0 | 0 | H | p-C17H35—Ph—* | H | H | H | H | H | H | p-C17H35—Ph—* | H |
| Compound example 44 | S | 0 | 0 | H | p-C18H37—Ph—* | H | H | H | H | H | H | p-C18H37—Ph—* | H |
| Compound example 45 | S | 1 | 1 | H | p-C5H11—Ph—* | H | H | H | H | H | H | p-C5H11—Ph—* | H |
| Compound example 46 | S | 1 | 1 | H | p-C6H13—Ph—* | H | H | H | H | H | H | p-C6H13—Ph—* | H |
| Compound example 47 | S | 1 | 1 | H | p-C7H15—Ph—* | H | H | H | H | H | H | p-C7H15—Ph—* | H |
| Compound example 48 | S | 1 | 1 | H | p-C8H17—Ph—* | H | H | H | H | H | H | p-C8H17—Ph—* | H |
| Compound example 49 | S | 1 | 1 | H | p-C9H19—Ph—* | H | H | H | H | H | H | p-C9H19—Ph—* | H |
| Compound example 50 | S | 1 | 1 | H | p-C10H21—Ph—* | H | H | H | H | H | H | p-C10H21—Ph—* | H |
| Compound example 51 | S | 1 | 1 | H | p-C11H23—Ph—* | H | H | H | H | H | H | p-C11H23—Ph—* | H |
| Compound example 52 | S | 1 | 1 | H | p-C12H25—Ph—* | H | H | H | H | H | H | p-C12H25—Ph—* | H |
| Compound example 53 | S | 1 | 1 | H | p-C13H27—Ph—* | H | H | H | H | H | H | p-C13H27—Ph—* | H |
| Compound example 54 | S | 1 | 1 | H | p-C14H29—Ph—* | H | H | H | H | H | H | p-C14H29—Ph—* | H |
| Compound example 55 | S | 1 | 1 | H | p-C15H31—Ph—* | H | H | H | H | H | H | p-C15H31—Ph—* | H |
| Compound example 56 | S | 1 | 1 | H | p-C16H33—Ph—* | H | H | H | H | H | H | p-C16H33—Ph—* | H |
| Compound example 57 | S | 1 | 1 | H | p-C17H35—Ph—* | H | H | H | H | H | H | p-C17H35—Ph—* | H |
| Compound example 58 | S | 1 | 1 | H | p-C18H37—Ph—* | H | H | H | H | H | H | p-C18H37—Ph—* | H |

TABLE 3

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 59 | S | 0 | 0 | H | 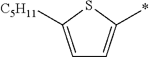 | H | H | H | H | H | H | 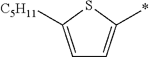 | H |
| Compound example 60 | S | 0 | 0 | H | 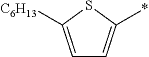 | H | H | H | H | H | H | 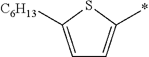 | H |
| Compound example 61 | S | 0 | 0 | H | 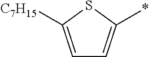 | H | H | H | H | H | H | 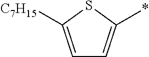 | H |
| Compound example 62 | S | 0 | 0 | H | 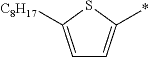 | H | H | H | H | H | H | 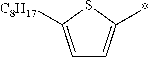 | H |
| Compound example 63 | S | 0 | 0 | H | 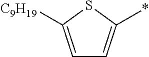 | H | H | H | H | H | H | 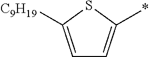 | H |
| Compound example 64 | S | 0 | 0 | H | 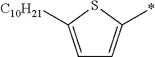 | H | H | H | H | H | H | 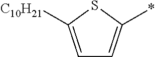 | H |
| Compound example 65 | S | 0 | 0 | H | 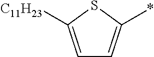 | H | H | H | H | H | H | 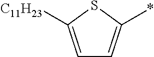 | H |
| Compound example 66 | S | 0 | 0 | H | 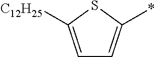 | H | H | H | H | H | H | 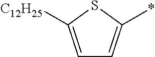 | H |
| Compound example 67 | S | 0 | 0 | H | 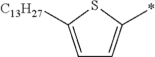 | H | H | H | H | H | H | 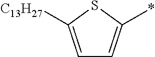 | H |
| Compound example 68 | S | 0 | 0 | H | 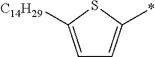 | H | H | H | H | H | H | 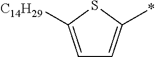 | H |

TABLE 4

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 69 | S | 0 | 0 | H | 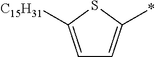 | H | H | H | H | H | H | 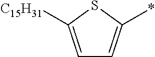 | H |
| Compound example 70 | S | 0 | 0 | H | 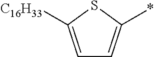 | H | H | H | H | H | H | 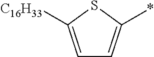 | H |
| Compound example 71 | S | 0 | 0 | H | 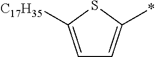 | H | H | H | H | H | H | 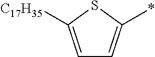 | H |
| Compound example 72 | S | 0 | 0 | H | 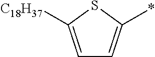 | H | H | H | H | H | H | 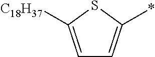 | H |
| Compound example 73 | S | 1 | 1 | H | 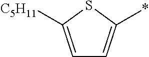 | H | H | H | H | H | H | 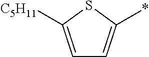 | H |
| Compound example 74 | S | 1 | 1 | H | 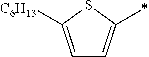 | H | H | H | H | H | H | 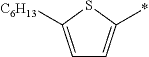 | H |
| Compound example 75 | S | 1 | 1 | H | 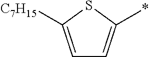 | H | H | H | H | H | H | 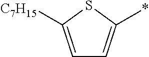 | H |
| Compound example 76 | S | 1 | 1 | H | 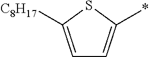 | H | H | H | H | H | H | 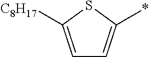 | H |
| Compound example 77 | S | 1 | 1 | H | 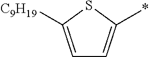 | H | H | H | H | H | H | 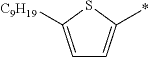 | H |
| Compound example 78 | S | 1 | 1 | H | 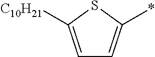 | H | H | H | H | H | H | 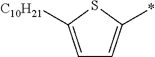 | H |

TABLE 5

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 79 | S | 1 | 1 | H | 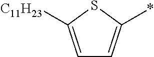 | H | H | H | H | H | H | 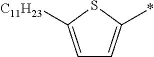 | H |
| Compound example 80 | S | 1 | 1 | H | 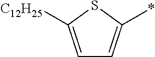 | H | H | H | H | H | H | 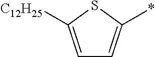 | H |
| Compound example 81 | S | 1 | 1 | H | 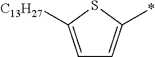 | H | H | H | H | H | H | 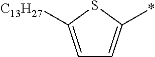 | H |
| Compound example 82 | S | 1 | 1 | H | 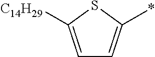 | H | H | H | H | H | H | 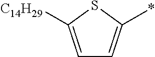 | H |
| Compound example 83 | S | 1 | 1 | H | 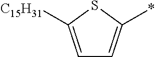 | H | H | H | H | H | H | 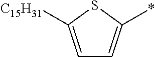 | H |
| Compound example 84 | S | 1 | 1 | H | 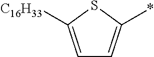 | H | H | H | H | H | H | 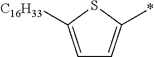 | H |
| Compound example 85 | S | 1 | 1 | H | 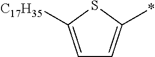 | H | H | H | H | H | H | 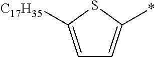 | H |
| Compound example 86 | S | 1 | 1 | H | 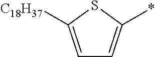 | H | H | H | H | H | H | 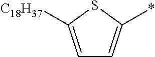 | H |

TABLE 6

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 87 | S | 0 | 0 | H | C5H11 | H | H | H | H | H | H | C6H13 | H |
| Compound example 88 | S | 0 | 0 | H | C5H11 | H | H | H | H | H | H | C7H15 | H |
| Compound example 89 | S | 0 | 0 | H | C5H11 | H | H | H | H | H | H | C8H17 | H |
| Compound example 90 | S | 0 | 0 | H | C5H11 | H | H | H | H | H | H | C9H19 | H |
| Compound example 91 | S | 0 | 0 | H | C5H11 | H | H | H | H | H | H | C10H21 | H |
| Compound example 92 | S | 0 | 0 | H | C5H11 | H | H | H | H | H | H | C11H23 | H |
| Compound example 93 | S | 0 | 0 | H | C5H11 | H | H | H | H | H | H | C12H25 | H |
| Compound example 94 | S | 0 | 0 | H | C5H11 | H | H | H | H | H | H | C13H27 | H |
| Compound example 95 | S | 0 | 0 | H | C5H11 | H | H | H | H | H | H | C14H29 | H |
| Compound example 96 | S | 0 | 0 | H | C5H11 | H | H | H | H | H | H | C15H31 | H |
| Compound example 97 | S | 0 | 0 | H | C5H11 | H | H | H | H | H | H | C16H33 | H |
| Compound example 98 | S | 0 | 0 | H | C5H11 | H | H | H | H | H | H | C17H35 | H |
| Compound example 99 | S | 0 | 0 | H | C5H11 | H | H | H | H | H | H | C18H37 | H |
| Compound example 100 | S | 0 | 0 | H | C6H13 | H | H | H | H | H | H | C5H11 | H |
| Compound example 101 | S | 0 | 0 | H | C6H13 | H | H | H | H | H | H | C7H15 | H |
| Compound example 102 | S | 0 | 0 | H | C6H13 | H | H | H | H | H | H | C8H17 | H |
| Compound example 103 | S | 0 | 0 | H | C6H13 | H | H | H | H | H | H | C9H19 | H |
| Compound example 104 | S | 0 | 0 | H | C6H13 | H | H | H | H | H | H | C10H21 | H |
| Compound example 105 | S | 0 | 0 | H | C6H13 | H | H | H | H | H | H | C11H23 | H |
| Compound example 106 | S | 0 | 0 | H | C6H13 | H | H | H | H | H | H | C12H25 | H |
| Compound example 107 | S | 0 | 0 | H | C6H13 | H | H | H | H | H | H | C13H27 | H |
| Compound example 108 | S | 0 | 0 | H | C6H13 | H | H | H | H | H | H | C14H29 | H |
| Compound example 109 | S | 0 | 0 | H | C6H13 | H | H | H | H | H | H | C15H31 | H |
| Compound example 110 | S | 0 | 0 | H | C6H13 | H | H | H | H | H | H | C16H33 | H |
| Compound example 111 | S | 0 | 0 | H | C6H13 | H | H | H | H | H | H | C17H35 | H |
| Compound example 112 | S | 0 | 0 | H | C6H13 | H | H | H | H | H | H | C18H37 | H |

TABLE 7

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 113 | S | 0 | 0 | H | C7H15 | H | H | H | H | H | H | C5H11 | H |
| Compound example 114 | S | 0 | 0 | H | C7H15 | H | H | H | H | H | H | C6H13 | H |
| Compound example 115 | S | 0 | 0 | H | C7H15 | H | H | H | H | H | H | C8H17 | H |
| Compound example 116 | S | 0 | 0 | H | C7H15 | H | H | H | H | H | H | C9H19 | H |
| Compound example 117 | S | 0 | 0 | H | C7H15 | H | H | H | H | H | H | C10H21 | H |
| Compound example 118 | S | 0 | 0 | H | C7H15 | H | H | H | H | H | H | C11H23 | H |
| Compound example 119 | S | 0 | 0 | H | C7H15 | H | H | H | H | H | H | C12H25 | H |
| Compound example 120 | S | 0 | 0 | H | C7H15 | H | H | H | H | H | H | C13H27 | H |
| Compound example 121 | S | 0 | 0 | H | C7H15 | H | H | H | H | H | H | C14H29 | H |
| Compound example 122 | S | 0 | 0 | H | C7H15 | H | H | H | H | H | H | C15H31 | H |
| Compound example 123 | S | 0 | 0 | H | C7H15 | H | H | H | H | H | H | C16H33 | H |
| Compound example 124 | S | 0 | 0 | H | C7H15 | H | H | H | H | H | H | C17H35 | H |
| Compound example 125 | S | 0 | 0 | H | C7H15 | H | H | H | H | H | H | C18H37 | H |
| Compound example 126 | S | 0 | 0 | H | C8H17 | H | H | H | H | H | H | C5H11 | H |
| Compound example 127 | S | 0 | 0 | H | C8H17 | H | H | H | H | H | H | C6H13 | H |
| Compound example 128 | S | 0 | 0 | H | C8H17 | H | H | H | H | H | H | C7H15 | H |
| Compound example 129 | S | 0 | 0 | H | C8H17 | H | H | H | H | H | H | C9H19 | H |
| Compound example 130 | S | 0 | 0 | H | C8H17 | H | H | H | H | H | H | C10H21 | H |
| Compound example 131 | S | 0 | 0 | H | C8H17 | H | H | H | H | H | H | C11H23 | H |
| Compound example 132 | S | 0 | 0 | H | C8H17 | H | H | H | H | H | H | C12H25 | H |
| Compound example 133 | S | 0 | 0 | H | C8H17 | H | H | H | H | H | H | C13H27 | H |
| Compound example 134 | S | 0 | 0 | H | C8H17 | H | H | H | H | H | H | C14H29 | H |
| Compound example 135 | S | 0 | 0 | H | C8H17 | H | H | H | H | H | H | C15H31 | H |
| Compound example 136 | S | 0 | 0 | H | C8H17 | H | H | H | H | H | H | C16H33 | H |
| Compound example 137 | S | 0 | 0 | H | C8H17 | H | H | H | H | H | H | C17H35 | H |
| Compound example 138 | S | 0 | 0 | H | C8H17 | H | H | H | H | H | H | C18H37 | H |

TABLE 8

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 139 | S | 0 | 0 | H | C9H19 | H | H | H | H | H | H | C5H11 | H |
| Compound example 140 | S | 0 | 0 | H | C9H19 | H | H | H | H | H | H | C6H13 | H |
| Compound example 141 | S | 0 | 0 | H | C9H19 | H | H | H | H | H | H | C7H15 | H |
| Compound example 142 | S | 0 | 0 | H | C9H19 | H | H | H | H | H | H | C8H17 | H |
| Compound example 143 | S | 0 | 0 | H | C9H19 | H | H | H | H | H | H | C10H21 | H |
| Compound example 144 | S | 0 | 0 | H | C9H19 | H | H | H | H | H | H | C11H23 | H |
| Compound example 145 | S | 0 | 0 | H | C9H19 | H | H | H | H | H | H | C12H25 | H |
| Compound example 146 | S | 0 | 0 | H | C9H19 | H | H | H | H | H | H | C13H27 | H |
| Compound example 147 | S | 0 | 0 | H | C9H19 | H | H | H | H | H | H | C14H29 | H |
| Compound example 148 | S | 0 | 0 | H | C9H19 | H | H | H | H | H | H | C15H31 | H |
| Compound example 149 | S | 0 | 0 | H | C9H19 | H | H | H | H | H | H | C16H33 | H |
| Compound example 150 | S | 0 | 0 | H | C9H19 | H | H | H | H | H | H | C17H35 | H |
| Compound example 151 | S | 0 | 0 | H | C9H19 | H | H | H | H | H | H | C18H37 | H |
| Compound example 152 | S | 0 | 0 | H | C10H21 | H | H | H | H | H | H | C5H11 | H |
| Compound example 153 | S | 0 | 0 | H | C10H21 | H | H | H | H | H | H | C6H13 | H |
| Compound example 154 | S | 0 | 0 | H | C10H21 | H | H | H | H | H | H | C7H15 | H |
| Compound example 155 | S | 0 | 0 | H | C10H21 | H | H | H | H | H | H | C8H17 | H |
| Compound example 156 | S | 0 | 0 | H | C10H21 | H | H | H | H | H | H | C9H19 | H |
| Compound example 157 | S | 0 | 0 | H | C10H21 | H | H | H | H | H | H | C11H23 | H |
| Compound example 158 | S | 0 | 0 | H | C10H21 | H | H | H | H | H | H | C12H25 | H |
| Compound example 159 | S | 0 | 0 | H | C10H21 | H | H | H | H | H | H | C13H27 | H |
| Compound example 160 | S | 0 | 0 | H | C10H21 | H | H | H | H | H | H | C14H29 | H |
| Compound example 161 | S | 0 | 0 | H | C10H21 | H | H | H | H | H | H | C15H31 | H |
| Compound example 162 | S | 0 | 0 | H | C10H21 | H | H | H | H | H | H | C16H33 | H |
| Compound example 163 | S | 0 | 0 | H | C10H21 | H | H | H | H | H | H | C17H35 | H |
| Compound example 164 | S | 0 | 0 | H | C10H21 | H | H | H | H | H | H | C18H37 | H |

TABLE 9

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 165 | S | 0 | 0 | H | C11H23 | H | H | H | H | H | H | C5H11 | H |
| Compound example 166 | S | 0 | 0 | H | C11H23 | H | H | H | H | H | H | C6H13 | H |
| Compound example 167 | S | 0 | 0 | H | C11H23 | H | H | H | H | H | H | C7H15 | H |
| Compound example 168 | S | 0 | 0 | H | C11H23 | H | H | H | H | H | H | C8H17 | H |
| Compound example 169 | S | 0 | 0 | H | C11H23 | H | H | H | H | H | H | C9H19 | H |
| Compound example 170 | S | 0 | 0 | H | C11H23 | H | H | H | H | H | H | C10H21 | H |
| Compound example 171 | S | 0 | 0 | H | C11H23 | H | H | H | H | H | H | C12H25 | H |
| Compound example 172 | S | 0 | 0 | H | C11H23 | H | H | H | H | H | H | C13H27 | H |
| Compound example 173 | S | 0 | 0 | H | C11H23 | H | H | H | H | H | H | C14H29 | H |
| Compound example 174 | S | 0 | 0 | H | C11H23 | H | H | H | H | H | H | C15H31 | H |
| Compound example 175 | S | 0 | 0 | H | C11H23 | H | H | H | H | H | H | C16H33 | H |
| Compound example 176 | S | 0 | 0 | H | C11H23 | H | H | H | H | H | H | C17H35 | H |
| Compound example 177 | S | 0 | 0 | H | C11H23 | H | H | H | H | H | H | C18H37 | H |
| Compound example 178 | S | 0 | 0 | H | C12H25 | H | H | H | H | H | H | C5H11 | H |
| Compound example 179 | S | 0 | 0 | H | C12H25 | H | H | H | H | H | H | C6H13 | H |
| Compound example 180 | S | 0 | 0 | H | C12H25 | H | H | H | H | H | H | C7H15 | H |
| Compound example 181 | S | 0 | 0 | H | C12H25 | H | H | H | H | H | H | C8H17 | H |
| Compound example 182 | S | 0 | 0 | H | C12H25 | H | H | H | H | H | H | C9H19 | H |
| Compound example 183 | S | 0 | 0 | H | C12H25 | H | H | H | H | H | H | C10H21 | H |
| Compound example 184 | S | 0 | 0 | H | C12H25 | H | H | H | H | H | H | C11H23 | H |
| Compound example 185 | S | 0 | 0 | H | C12H25 | H | H | H | H | H | H | C13H27 | H |
| Compound example 186 | S | 0 | 0 | H | C12H25 | H | H | H | H | H | H | C14H29 | H |
| Compound example 187 | S | 0 | 0 | H | C12H25 | H | H | H | H | H | H | C15H31 | H |
| Compound example 188 | S | 0 | 0 | H | C12H25 | H | H | H | H | H | H | C16H33 | H |
| Compound example 189 | S | 0 | 0 | H | C12H25 | H | H | H | H | H | H | C17H35 | H |
| Compound example 190 | S | 0 | 0 | H | C12H25 | H | H | H | H | H | H | C18H37 | H |

TABLE 10

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 191 | S | 0 | 0 | H | C13H27 | H | H | H | H | H | H | C5H11 | H |
| Compound example 192 | S | 0 | 0 | H | C13H27 | H | H | H | H | H | H | C6H13 | H |
| Compound example 193 | S | 0 | 0 | H | C13H27 | H | H | H | H | H | H | C7H15 | H |
| Compound example 194 | S | 0 | 0 | H | C13H27 | H | H | H | H | H | H | C8H17 | H |
| Compound example 195 | S | 0 | 0 | H | C13H27 | H | H | H | H | H | H | C9H19 | H |
| Compound example 196 | S | 0 | 0 | H | C13H27 | H | H | H | H | H | H | C10H21 | H |
| Compound example 197 | S | 0 | 0 | H | C13H27 | H | H | H | H | H | H | C11H23 | H |
| Compound example 198 | S | 0 | 0 | H | C13H27 | H | H | H | H | H | H | C12H25 | H |
| Compound example 199 | S | 0 | 0 | H | C13H27 | H | H | H | H | H | H | C14H29 | H |
| Compound example 200 | S | 0 | 0 | H | C13H27 | H | H | H | H | H | H | C15H31 | H |
| Compound example 201 | S | 0 | 0 | H | C13H27 | H | H | H | H | H | H | C16H33 | H |
| Compound example 202 | S | 0 | 0 | H | C13H27 | H | H | H | H | H | H | C17H35 | H |
| Compound example 203 | S | 0 | 0 | H | C13H27 | H | H | H | H | H | H | C18H37 | H |
| Compound example 204 | S | 0 | 0 | H | C14H29 | H | H | H | H | H | H | C5H11 | H |
| Compound example 205 | S | 0 | 0 | H | C14H29 | H | H | H | H | H | H | C6H13 | H |
| Compound example 206 | S | 0 | 0 | H | C14H29 | H | H | H | H | H | H | C7H15 | H |
| Compound example 207 | S | 0 | 0 | H | C14H29 | H | H | H | H | H | H | C8H17 | H |
| Compound example 208 | S | 0 | 0 | H | C14H29 | H | H | H | H | H | H | C9H19 | H |
| Compound example 209 | S | 0 | 0 | H | C14H29 | H | H | H | H | H | H | C10H21 | H |
| Compound example 210 | S | 0 | 0 | H | C14H29 | H | H | H | H | H | H | C11H23 | H |
| Compound example 211 | S | 0 | 0 | H | C14H29 | H | H | H | H | H | H | C12H25 | H |
| Compound example 212 | S | 0 | 0 | H | C14H29 | H | H | H | H | H | H | C13H27 | H |
| Compound example 213 | S | 0 | 0 | H | C14H29 | H | H | H | H | H | H | C15H31 | H |
| Compound example 214 | S | 0 | 0 | H | C14H29 | H | H | H | H | H | H | C16H33 | H |
| Compound example 215 | S | 0 | 0 | H | C14H29 | H | H | H | H | H | H | C17H35 | H |
| Compound example 216 | S | 0 | 0 | H | C14H29 | H | H | H | H | H | H | C18H37 | H |

TABLE 11

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 217 | S | 0 | 0 | H | C15H31 | H | H | H | H | H | H | C5H11 | H |
| Compound example 218 | S | 0 | 0 | H | C15H31 | H | H | H | H | H | H | C6H13 | H |
| Compound example 219 | S | 0 | 0 | H | C15H31 | H | H | H | H | H | H | C7H15 | H |
| Compound example 220 | S | 0 | 0 | H | C15H31 | H | H | H | H | H | H | C8H17 | H |
| Compound example 221 | S | 0 | 0 | H | C15H31 | H | H | H | H | H | H | C9H19 | H |
| Compound example 222 | S | 0 | 0 | H | C15H31 | H | H | H | H | H | H | C10H21 | H |
| Compound example 223 | S | 0 | 0 | H | C15H31 | H | H | H | H | H | H | C11H23 | H |
| Compound example 224 | S | 0 | 0 | H | C15H31 | H | H | H | H | H | H | C12H25 | H |
| Compound example 225 | S | 0 | 0 | H | C15H31 | H | H | H | H | H | H | C13H27 | H |
| Compound example 226 | S | 0 | 0 | H | C15H31 | H | H | H | H | H | H | C14H29 | H |
| Compound example 227 | S | 0 | 0 | H | C15H31 | H | H | H | H | H | H | C16H33 | H |
| Compound example 228 | S | 0 | 0 | H | C15H31 | H | H | H | H | H | H | C17H35 | H |
| Compound example 229 | S | 0 | 0 | H | C15H31 | H | H | H | H | H | H | C18H37 | H |
| Compound example 230 | S | 0 | 0 | H | C16H33 | H | H | H | H | H | H | C5H11 | H |
| Compound example 231 | S | 0 | 0 | H | C16H33 | H | H | H | H | H | H | C6H13 | H |
| Compound example 232 | S | 0 | 0 | H | C16H33 | H | H | H | H | H | H | C7H15 | H |
| Compound example 233 | S | 0 | 0 | H | C16H33 | H | H | H | H | H | H | C8H17 | H |
| Compound example 234 | S | 0 | 0 | H | C16H33 | H | H | H | H | H | H | C9H19 | H |
| Compound example 235 | S | 0 | 0 | H | C16H33 | H | H | H | H | H | H | C10H21 | H |
| Compound example 236 | S | 0 | 0 | H | C16H33 | H | H | H | H | H | H | C11H23 | H |
| Compound example 237 | S | 0 | 0 | H | C16H33 | H | H | H | H | H | H | C12H25 | H |
| Compound example 238 | S | 0 | 0 | H | C16H33 | H | H | H | H | H | H | C13H27 | H |
| Compound example 239 | S | 0 | 0 | H | C16H33 | H | H | H | H | H | H | C14H29 | H |
| Compound example 240 | S | 0 | 0 | H | C16H33 | H | H | H | H | H | H | C15H31 | H |
| Compound example 241 | S | 0 | 0 | H | C16H33 | H | H | H | H | H | H | C17H35 | H |
| Compound example 242 | S | 0 | 0 | H | C16H33 | H | H | H | H | H | H | C18H37 | H |

TABLE 12

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 243 | S | 0 | 0 | H | C17H35 | H | H | H | H | H | H | C5H11 | H |
| Compound example 244 | S | 0 | 0 | H | C17H35 | H | H | H | H | H | H | C6H13 | H |
| Compound example 245 | S | 0 | 0 | H | C17H35 | H | H | H | H | H | H | C7H15 | H |
| Compound example 246 | S | 0 | 0 | H | C17H35 | H | H | H | H | H | H | C8H17 | H |
| Compound example 247 | S | 0 | 0 | H | C17H35 | H | H | H | H | H | H | C9H19 | H |
| Compound example 248 | S | 0 | 0 | H | C17H35 | H | H | H | H | H | H | C10H21 | H |
| Compound example 249 | S | 0 | 0 | H | C17H35 | H | H | H | H | H | H | C11H23 | H |
| Compound example 250 | S | 0 | 0 | H | C17H35 | H | H | H | H | H | H | C12H25 | H |
| Compound example 251 | S | 0 | 0 | H | C17H35 | H | H | H | H | H | H | C13H27 | H |
| Compound example 252 | S | 0 | 0 | H | C17H35 | H | H | H | H | H | H | C14H29 | H |
| Compound example 253 | S | 0 | 0 | H | C17H35 | H | H | H | H | H | H | C15H31 | H |
| Compound example 254 | S | 0 | 0 | H | C17H35 | H | H | H | H | H | H | C16H33 | H |
| Compound example 255 | S | 0 | 0 | H | C17H35 | H | H | H | H | H | H | C18H37 | H |
| Compound example 256 | S | 0 | 0 | H | C18H37 | H | H | H | H | H | H | C6H13 | H |
| Compound example 257 | S | 0 | 0 | H | C18H37 | H | H | H | H | H | H | C7H15 | H |
| Compound example 258 | S | 0 | 0 | H | C18H37 | H | H | H | H | H | H | C8H17 | H |
| Compound example 259 | S | 0 | 0 | H | C18H37 | H | H | H | H | H | H | C9H19 | H |
| Compound example 260 | S | 0 | 0 | H | C18H37 | H | H | H | H | H | H | C10H21 | H |
| Compound example 261 | S | 0 | 0 | H | C18H37 | H | H | H | H | H | H | C11H23 | H |
| Compound example 262 | S | 0 | 0 | H | C18H37 | H | H | H | H | H | H | C12H25 | H |
| Compound example 263 | S | 0 | 0 | H | C18H37 | H | H | H | H | H | H | C13H27 | H |
| Compound example 264 | S | 0 | 0 | H | C18H37 | H | H | H | H | H | H | C14H29 | H |
| Compound example 265 | S | 0 | 0 | H | C18H37 | H | H | H | H | H | H | C15H31 | H |
| Compound example 266 | S | 0 | 0 | H | C18H37 | H | H | H | H | H | H | C16H33 | H |
| Compound example 267 | S | 0 | 0 | H | C18H37 | H | H | H | H | H | H | C17H35 | H |

TABLE 13

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 268 | S | 0 | 0 | H | C8H17 | H | H | H | H | H | H | p-C5H11—Ph—* | H |
| Compound example 269 | S | 0 | 0 | H | C8H17 | H | H | H | H | H | H | p-C6H13—Ph—* | H |
| Compound example 270 | S | 0 | 0 | H | C8H17 | H | H | H | H | H | H | p-C7H15—Ph—* | H |
| Compound example 271 | S | 0 | 0 | H | C8H17 | H | H | H | H | H | H | p-C8H17—Ph—* | H |
| Compound example 272 | S | 0 | 0 | H | C8H17 | H | H | H | H | H | H | p-C9H19—Ph—* | H |
| Compound example 273 | S | 0 | 0 | H | C8H17 | H | H | H | H | H | H | p-C10H21—Ph—* | H |
| Compound example 274 | S | 0 | 0 | H | C8H17 | H | H | H | H | H | H | p-C11H23—Ph—* | H |
| Compound example 275 | S | 0 | 0 | H | C8H17 | H | H | H | H | H | H | p-C12H25—Ph—* | H |
| Compound example 276 | S | 0 | 0 | H | C8H17 | H | H | H | H | H | H | p-C13H27—Ph—* | H |
| Compound example 277 | S | 0 | 0 | H | C8H17 | H | H | H | H | H | H | p-C14H29—Ph—* | H |
| Compound example 278 | S | 0 | 0 | H | C8H17 | H | H | H | H | H | H | p-C15H31—Ph—* | H |
| Compound example 279 | S | 0 | 0 | H | C8H17 | H | H | H | H | H | H | p-C16H33—Ph—* | H |
| Compound example 280 | S | 0 | 0 | H | C8H17 | H | H | H | H | H | H | p-C17H35—Ph—* | H |
| Compound example 281 | S | 0 | 0 | H | C8H17 | H | H | H | H | H | H | p-C18H37—Ph—* | H |
| Compound example 282 | S | 0 | 0 | H | C8H17 | H | H | H | H | H | H | 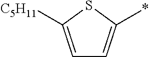 | H |
| Compound example 283 | S | 0 | 0 | H | C8H17 | H | H | H | H | H | H | 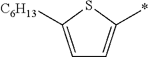 | H |
| Compound example 284 | S | 0 | 0 | H | C8H17 | H | H | H | H | H | H | 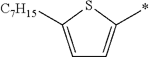 | H |
| Compound example 285 | S | 0 | 0 | H | C8H17 | H | H | H | H | H | H | 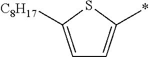 | H |

TABLE 14

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 286 | S | 0 | 0 | H | C8H17 | H | H | H | H | H | H | 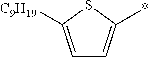 | H |
| Compound example 287 | S | 0 | 0 | H | C8H17 | H | H | H | H | H | H | 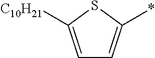 | H |
| Compound example 288 | S | 0 | 0 | H | C8H17 | H | H | H | H | H | H | 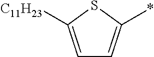 | H |
| Compound example 289 | S | 0 | 0 | H | C8H17 | H | H | H | H | H | H | 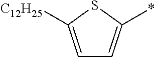 | H |
| Compound example 290 | S | 0 | 0 | H | C8H17 | H | H | H | H | H | H | 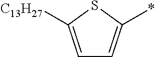 | H |
| Compound example 291 | S | 0 | 0 | H | C8H17 | H | H | H | H | H | H | 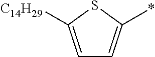 | H |
| Compound example 292 | S | 0 | 0 | H | C8H17 | H | H | H | H | H | H | 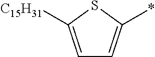 | H |
| Compound example 293 | S | 0 | 0 | H | C8H17 | H | H | H | H | H | H | 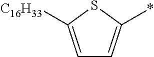 | H |
| Compound example 294 | S | 0 | 0 | H | C8H17 | H | H | H | H | H | H | 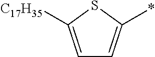 | H |
| Compound example 295 | S | 0 | 0 | H | C8H17 | H | H | H | H | H | H | 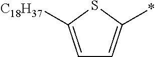 | H |

TABLE 15

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 296 | S | 0 | 0 | H | 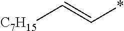 | H | H | H | H | H | H | 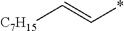 | H |
| Compound example 297 | S | 0 | 0 | H | 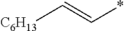 | H | H | H | H | H | H | 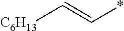 | H |
| Compound example 298 | S | 0 | 0 | H | 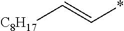 | H | H | H | H | H | H | 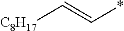 | H |
| Compound example 299 | S | 0 | 0 | H | 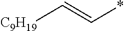 | H | H | H | H | H | H | 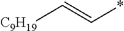 | H |
| Compound example 300 | S | 0 | 0 | H | 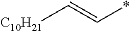 | H | H | H | H | H | H | 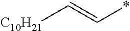 | H |
| Compound example 301 | S | 0 | 0 | H | 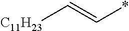 | H | H | H | H | H | H | 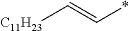 | H |
| Compound example 302 | S | 0 | 0 | H | 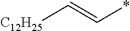 | H | H | H | H | H | H | 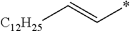 | H |
| Compound example 303 | S | 0 | 0 | H | 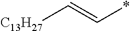 | H | H | H | H | H | H | 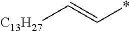 | H |
| Compound example 304 | S | 0 | 0 | H | 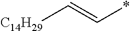 | H | H | H | H | H | H | 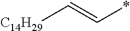 | H |
| Compound example 305 | S | 0 | 0 | H | 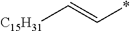 | H | H | H | H | H | H | 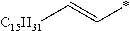 | H |

TABLE 16

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 306 | S | 0 | 0 | H | 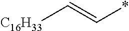 | H | H | H | H | H | H | 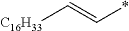 | H |
| Compound example 307 | S | 0 | 0 | H | 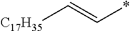 | H | H | H | H | H | H | 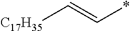 | H |
| Compound example 308 | S | 0 | 0 | H | 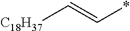 | H | H | H | H | H | H | 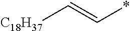 | H |
| Compound example 309 | S | 0 | 0 | H | 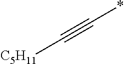 | H | H | H | H | H | H | 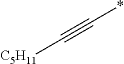 | H |
| Compound example 310 | S | 0 | 0 | H | 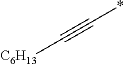 | H | H | H | H | H | H | 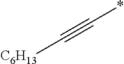 | H |
| Compound example 311 | S | 0 | 0 | H | 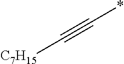 | H | H | H | H | H | H | 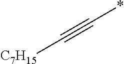 | H |
| Compound example 312 | S | 0 | 0 | H | 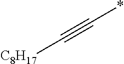 | H | H | H | H | H | H | 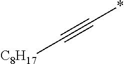 | H |
| Compound example 313 | S | 0 | 0 | H | 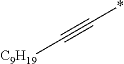 | H | H | H | H | H | H | 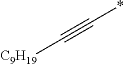 | H |
| Compound example 314 | S | 0 | 0 | H | 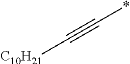 | H | H | H | H | H | H | 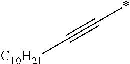 | H |
| Compound example 315 | S | 0 | 0 | H | 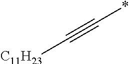 | H | H | H | H | H | H | 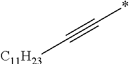 | H |

TABLE 17

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 316 | S | 0 | 0 | H | 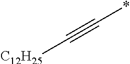 | H | H | H | H | H | H | 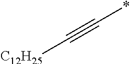 | H |
| Compound example 317 | S | 0 | 0 | H | 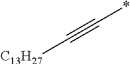 | H | H | H | H | H | H | 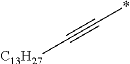 | H |
| Compound example 318 | S | 0 | 0 | H | 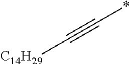 | H | H | H | H | H | H | 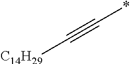 | H |
| Compound example 319 | S | 0 | 0 | H | 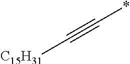 | H | H | H | H | H | H | 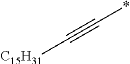 | H |
| Compound example 320 | S | 0 | 0 | H | 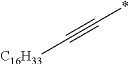 | H | H | H | H | H | H | 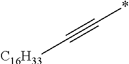 | H |
| Compound example 321 | S | 0 | 0 | H | 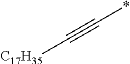 | H | H | H | H | H | H | 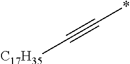 | H |
| Compound example 322 | S | 0 | 0 | H | 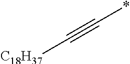 | H | H | H | H | H | H | 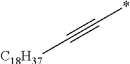 | H |

TABLE 18

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 323 | S | 0 | 0 | H | C₅H₁₁O | H | H | H | H | H | H | C₅H₁₁O | H |
| Compound example 324 | S | 0 | 0 | H | C₆H₁₃O | H | H | H | H | H | H | C₆H₁₃O | H |
| Compound example 325 | S | 0 | 0 | H | C₇H₁₅O | H | H | H | H | H | H | C₇H₁₅O | H |
| Compound example 326 | S | 0 | 0 | H | C₈H₁₇O | H | H | H | H | H | H | C₈H₁₇O | H |
| Compound example 327 | S | 0 | 0 | H | C₉H₁₉O | H | H | H | H | H | H | C₉H₁₉O | H |
| Compound example 328 | S | 0 | 0 | H | C₁₀H₂₁O | H | H | H | H | H | H | C₁₀H₂₁O | H |
| Compound example 329 | S | 0 | 0 | H | C₁₁H₂₃O | H | H | H | H | H | H | C₁₁H₂₃O | H |
| Compound example 330 | S | 0 | 0 | H | C₁₂H₂₅O | H | H | H | H | H | H | C₁₂H₂₅O | H |
| Compound example 331 | S | 0 | 0 | H | C₁₃H₂₇O | H | H | H | H | H | H | C₁₃H₂₇O | H |
| Compound example 332 | S | 0 | 0 | H | C₁₄H₂₉O | H | H | H | H | H | H | C₁₄H₂₉O | H |
| Compound example 333 | S | 0 | 0 | H | C₁₅H₃₁O | H | H | H | H | H | H | C₁₅H₃₁O | H |
| Compound example 334 | S | 0 | 0 | H | C₁₆H₃₃O | H | H | H | H | H | H | C₁₆H₃₃O | H |
| Compound example 335 | S | 0 | 0 | H | C₁₇H₃₅O | H | H | H | H | H | H | C₁₇H₃₅O | H |
| Compound example 336 | S | 0 | 0 | H | C₁₈H₃₇O | H | H | H | H | H | H | C₁₈H₃₇O | H |
| Compound example 337 | S | 0 | 1 | H | C5H11 | H | H | H | H | H | H | C5H11 | H |
| Compound example 338 | S | 0 | 1 | H | C6H13 | H | H | H | H | H | H | C6H13 | H |
| Compound example 339 | S | 0 | 1 | H | C7H15 | H | H | H | H | H | H | C7H15 | H |
| Compound example 340 | S | 0 | 1 | H | C8H17 | H | H | H | H | H | H | C8H17 | H |
| Compound example 341 | S | 0 | 1 | H | C9H19 | H | H | H | H | H | H | C9H19 | H |
| Compound example 342 | S | 0 | 1 | H | C10H21 | H | H | H | H | H | H | C10H21 | H |
| Compound example 343 | S | 0 | 1 | H | C11H23 | H | H | H | H | H | H | C11H23 | H |
| Compound example 344 | S | 0 | 1 | H | C12H25 | H | H | H | H | H | H | C12H25 | H |
| Compound example 345 | S | 0 | 1 | H | C13H27 | H | H | H | H | H | H | C13H27 | H |
| Compound example 346 | S | 0 | 1 | H | C14H29 | H | H | H | H | H | H | C14H29 | H |
| Compound example 347 | S | 0 | 1 | H | C15H31 | H | H | H | H | H | H | C15H31 | H |
| Compound example 348 | S | 0 | 1 | H | C16H33 | H | H | H | H | H | H | C16H33 | H |
| Compound example 349 | S | 0 | 1 | H | C17H35 | H | H | H | H | H | H | C17H35 | H |
| Compound example 350 | S | 0 | 1 | H | C18H37 | H | H | H | H | H | H | C18H37 | H |

TABLE 19

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 351 | S | 0 | 0 | H | H | H | H | H | H | H | H | C5H11 | H |
| Compound example 352 | S | 0 | 0 | H | H | H | H | H | H | H | H | C6H13 | H |
| Compound example 353 | S | 0 | 0 | H | H | H | H | H | H | H | H | C7H15 | H |
| Compound example 354 | S | 0 | 0 | H | H | H | H | H | H | H | H | C8H17 | H |
| Compound example 355 | S | 0 | 0 | H | H | H | H | H | H | H | H | C9H19 | H |
| Compound example 356 | S | 0 | 0 | H | H | H | H | H | H | H | H | C10H21 | H |
| Compound example 357 | S | 0 | 0 | H | H | H | H | H | H | H | H | C11H23 | H |
| Compound example 358 | S | 0 | 0 | H | H | H | H | H | H | H | H | C12H25 | H |
| Compound example 359 | S | 0 | 0 | H | H | H | H | H | H | H | H | C13H27 | H |
| Compound example 360 | S | 0 | 0 | H | H | H | H | H | H | H | H | C14H29 | H |
| Compound example 361 | S | 0 | 0 | H | H | H | H | H | H | H | H | C15H31 | H |
| Compound example 362 | S | 0 | 0 | H | H | H | H | H | H | H | H | C16H33 | H |
| Compound example 363 | S | 0 | 0 | H | H | H | H | H | H | H | H | C17H35 | H |
| Compound example 364 | S | 0 | 0 | H | H | H | H | H | H | H | H | C18H37 | H |
| Compound example 365 | S | 0 | 0 | H | H | H | H | H | H | H | H | p-C5H11—Ph—* | H |
| Compound example 366 | S | 0 | 0 | H | H | H | H | H | H | H | H | p-C6H13—Ph—* | H |
| Compound example 367 | S | 0 | 0 | H | H | H | H | H | H | H | H | p-C7H15—Ph—* | H |
| Compound example 368 | S | 0 | 0 | H | H | H | H | H | H | H | H | p-C8H17—Ph—* | H |
| Compound example 369 | S | 0 | 0 | H | H | H | H | H | H | H | H | p-C9H19—Ph—* | H |
| Compound example 370 | S | 0 | 0 | H | H | H | H | H | H | H | H | p-C10H21—Ph—* | H |
| Compound example 371 | S | 0 | 0 | H | H | H | H | H | H | H | H | p-C11H23—Ph—* | H |
| Compound example 372 | S | 0 | 0 | H | H | H | H | H | H | H | H | p-C12H25—Ph—* | H |
| Compound example 373 | S | 0 | 0 | H | H | H | H | H | H | H | H | p-C13H27—Ph—* | H |
| Compound example 374 | S | 0 | 0 | H | H | H | H | H | H | H | H | p-C14H29—Ph—* | H |
| Compound example 375 | S | 0 | 0 | H | H | H | H | H | H | H | H | p-C15H31—Ph—* | H |
| Compound example 376 | S | 0 | 0 | H | H | H | H | H | H | H | H | p-C16H33—Ph—* | H |
| Compound example 377 | S | 0 | 0 | H | H | H | H | H | H | H | H | p-C17H35—Ph—* | H |
| Compound example 378 | S | 0 | 0 | H | H | H | H | H | H | H | H | p-C18H37—Ph—* | H |

TABLE 20

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 379 | S | 0 | 0 | H | H | H | H | H | H | H | H | 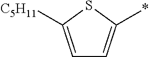 | H |
| Compound example 380 | S | 0 | 0 | H | H | H | H | H | H | H | H | 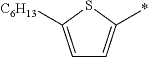 | H |
| Compound example 381 | S | 0 | 0 | H | H | H | H | H | H | H | H | 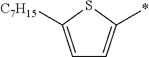 | H |
| Compound example 382 | S | 0 | 0 | H | H | H | H | H | H | H | H | 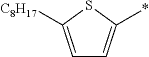 | H |
| Compound example 383 | S | 0 | 0 | H | H | H | H | H | H | H | H | 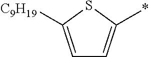 | H |
| Compound example 384 | S | 0 | 0 | H | H | H | H | H | H | H | H | 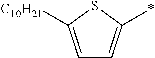 | H |
| Compound example 385 | S | 0 | 0 | H | H | H | H | H | H | H | H | 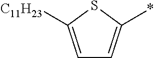 | H |
| Compound example 386 | S | 0 | 0 | H | H | H | H | H | H | H | H | 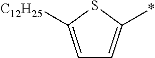 | H |

TABLE 21

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound example 387 | S | 0 | 0 | H | H | H | H | H | H | H | H | 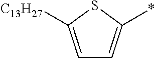 | H |
| Compound example 388 | S | 0 | 0 | H | H | H | H | H | H | H | H | 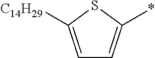 | H |
| Compound example 389 | S | 0 | 0 | H | H | H | H | H | H | H | H | 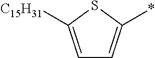 | H |
| Compound example 390 | S | 0 | 0 | H | H | H | H | H | H | H | H | 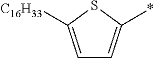 | H |
| Compound example 391 | S | 0 | 0 | H | H | H | H | H | H | H | H | 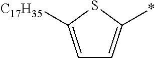 | H |
| Compound example 392 | S | 0 | 0 | H | H | H | H | H | H | H | H | 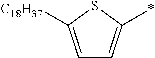 | H |

TABLE 22

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound | S | 0 | 0 | H | H | C5H11 | H | H | H | H | C5H11 | H | H |
| example 393 | | | | | | | | | | | | | |
| Compound | S | 0 | 0 | H | H | C6H13 | H | H | H | H | C6H13 | H | H |
| example 394 | | | | | | | | | | | | | |
| Compound | S | 0 | 0 | H | H | C7H15 | H | H | H | H | C7H15 | H | H |
| example 395 | | | | | | | | | | | | | |
| Compound | S | 0 | 0 | H | H | C8H17 | H | H | H | H | C8H17 | H | H |
| example 396 | | | | | | | | | | | | | |
| Compound | S | 0 | 0 | H | H | C9H19 | H | H | H | H | C9H19 | H | H |
| example 397 | | | | | | | | | | | | | |
| Compound | S | 0 | 0 | H | H | C10H21 | H | H | H | H | C10H21 | H | H |
| example 398 | | | | | | | | | | | | | |
| Compound | S | 0 | 0 | H | H | C11H23 | H | H | H | H | C11H23 | H | H |
| example 399 | | | | | | | | | | | | | |
| Compound | S | 0 | 0 | H | H | C12H25 | H | H | H | H | C12H25 | H | H |
| example 400 | | | | | | | | | | | | | |
| Compound | S | 0 | 0 | H | H | C13H27 | H | H | H | H | C13H27 | H | H |
| example 401 | | | | | | | | | | | | | |
| Compound | S | 0 | 0 | H | H | C14H29 | H | H | H | H | C14H29 | H | H |
| example 402 | | | | | | | | | | | | | |
| Compound | S | 0 | 0 | H | H | C15H31 | H | H | H | H | C15H31 | H | H |
| example 403 | | | | | | | | | | | | | |
| Compound | S | 0 | 0 | H | H | C16H33 | H | H | H | H | C16H33 | H | H |
| example 404 | | | | | | | | | | | | | |
| Compound | S | 0 | 0 | H | H | C17H35 | H | H | H | H | C17H35 | H | H |
| example 405 | | | | | | | | | | | | | |
| Compound | S | 0 | 0 | H | H | C18H37 | H | H | H | H | C18H37 | H | H |
| example 406 | | | | | | | | | | | | | |
| Compound | S | 0 | 0 | H | 3,7-Dimethyloctyl | H | H | H | H | H | H | 3,7-Dimethyloctyl | H |
| example 407 | | | | | | | | | | | | | |
| Compound | S | 0 | 0 | H | 2-Ethylhexyl | H | H | H | H | H | H | 2-Ethylhexyl | H |
| example 408 | | | | | | | | | | | | | |
| Compound example 409 | S | 0 | 0 | H | 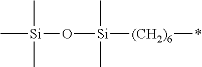 | H | H | H | H | H | H | 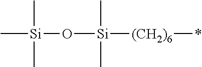 | H |
| Compound example 410 | S | 0 | 0 | H | H | H | H | H | H | H | H | 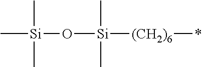 | H |
| Compound example 411 | S | 0 | 0 | H | 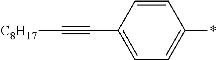 | H | H | H | H | H | H | 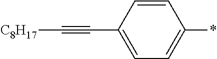 | H |
| Compound example 412 | S | 0 | 0 | H | 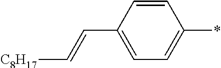 | H | H | H | H | H | H | 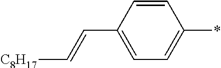 | H |

TABLE 23

| | X | p | q | R¹ | R² | R³ | R⁴ | R⁵ | R⁶ | R⁷ | R⁸ | R⁹ | R¹⁰ |
|---|---|---|---|---|---|---|---|---|---|---|---|---|---|
| Compound | O | 0 | 0 | H | C5H11 | H | H | H | H | H | H | C5H11 | H |
| example 413 | | | | | | | | | | | | | |
| Compound | O | 0 | 0 | H | C6H13 | H | H | H | H | H | H | C6H13 | H |
| example 414 | | | | | | | | | | | | | |
| Compound | O | 0 | 0 | H | C7H15 | H | H | H | H | H | H | C7H15 | H |
| example 415 | | | | | | | | | | | | | |
| Compound | O | 0 | 0 | H | C8H17 | H | H | H | H | H | H | C8H17 | H |
| example 416 | | | | | | | | | | | | | |
| Compound | O | 0 | 0 | H | C9H19 | H | H | H | H | H | H | C9H19 | H |
| example 417 | | | | | | | | | | | | | |
| Compound | O | 0 | 0 | H | C10H21 | H | H | H | H | H | H | C10H21 | H |
| example 418 | | | | | | | | | | | | | |
| Compound | O | 0 | 0 | H | C11H23 | H | H | H | H | H | H | C11H23 | H |
| example 419 | | | | | | | | | | | | | |
| Compound | O | 0 | 0 | H | C12H25 | H | H | H | H | H | H | C12H25 | H |
| example 420 | | | | | | | | | | | | | |
| Compound | O | 0 | 0 | H | C13H27 | H | H | H | H | H | H | C13H27 | H |
| example 421 | | | | | | | | | | | | | |
| Compound | O | 0 | 0 | H | C14H29 | H | H | H | H | H | H | C14H29 | H |
| example 422 | | | | | | | | | | | | | |
| Compound | O | 0 | 0 | H | C15H31 | H | H | H | H | H | H | C15H31 | H |
| example 423 | | | | | | | | | | | | | |
| Compound | O | 0 | 0 | H | C16H33 | H | H | H | H | H | H | C16H33 | H |
| example 424 | | | | | | | | | | | | | |
| Compound | O | 0 | 0 | H | C17H35 | H | H | H | H | H | H | C17H35 | H |
| example 425 | | | | | | | | | | | | | |
| Compound | O | 0 | 0 | H | C18H37 | H | H | H | H | H | H | C18H37 | H |
| example 426 | | | | | | | | | | | | | |
| Compound example 427 | S | 0 | 0 | H | 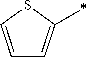 [SMILES: *c1cccs1] | H | H | 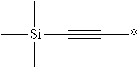 [SMILES: *C#C[Si](C)(C)C] | 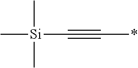 [SMILES: *C#C[Si](C)(C)C] | H | H | 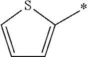 [SMILES: *c1cccs1] | H |
| Compound example 428 | S | 0 | 0 | H | Ph | H | H | 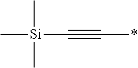 [SMILES: *C#C[Si](C)(C)C] | 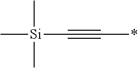 [SMILES: *C#C[Si](C)(C)C] | H | H | Ph | H |
| Compound | S | 0 | 0 | C5H11 | H | H | H | H | H | H | H | H | C5H11 |
| example 429 | | | | | | | | | | | | | |
| Compound | S | 0 | 0 | C6H13 | H | H | H | H | H | H | H | H | C6H13 |
| example 430 | | | | | | | | | | | | | |
| Compound | S | 0 | 0 | C7H15 | H | H | H | H | H | H | H | H | C7H15 |
| example 431 | | | | | | | | | | | | | |
| Compound | S | 0 | 0 | C8H17 | H | H | H | H | H | H | H | H | C8H17 |
| example 432 | | | | | | | | | | | | | |
| Compound | S | 0 | 0 | C9H19 | H | H | H | H | H | H | H | H | C9H19 |
| example 433 | | | | | | | | | | | | | |
| Compound | S | 0 | 0 | C10H21 | H | H | H | H | H | H | H | H | C10H21 |
| example 434 | | | | | | | | | | | | | |
| Compound | S | 0 | 0 | C11H23 | H | H | H | H | H | H | H | H | C11H23 |
| example 435 | | | | | | | | | | | | | |
| Compound | S | 0 | 0 | C12H25 | H | H | H | H | H | H | H | H | C12H25 |
| example 436 | | | | | | | | | | | | | |
| Compound | S | 0 | 0 | C13H27 | H | H | H | H | H | H | H | H | C13H27 |
| example 437 | | | | | | | | | | | | | |
| Compound | S | 0 | 0 | C14H29 | H | H | H | H | H | H | H | H | C14H29 |
| example 438 | | | | | | | | | | | | | |
| Compound | S | 0 | 0 | C15H31 | H | H | H | H | H | H | H | H | C15H31 |
| example 439 | | | | | | | | | | | | | |
| Compound | S | 0 | 0 | C16H33 | H | H | H | H | H | H | H | H | C16H33 |
| example 440 | | | | | | | | | | | | | |
| Compound | S | 0 | 0 | C17H35 | H | H | H | H | H | H | H | H | C17H35 |
| example 441 | | | | | | | | | | | | | |
| Compound | S | 0 | 0 | C18H37 | H | H | H | H | H | H | H | H | C18H37 |
| example 442 | | | | | | | | | | | | | |

The compound represented by the Formula (1) may have a repeating structure and may be a low-molecular weight compound or a polymer compound. In a case where the compound represented by Formula (1) is a low-molecular weight compound, the molecular weight thereof is preferably equal to or less than 3,000, more preferably equal to or less than 2,000, even more preferably equal to or less than 1,000, and particularly preferably equal to or less than 850. It is preferable that the molecular Weight is equal to or less than the upper limit described above because then the solubility in a solvent can be improved.

In contrast, from the viewpoint of the stability of the film quality, the molecular weight is preferably equal to or greater than 400, more preferably equal to or greater than 450, and even more preferably equal to or greater than 500.

Furthermore, in a case where the compound represented by Formula (1) is a polymer compound having a repeating structure, the weight average molecular weight thereof is preferably equal to or greater than 30,000, more preferably equal to or greater than 50,000, and even more preferably equal to or greater than 100,000. In a case where the compound represented by Formula (1) is a polymer compound having a repeating structure, it is preferable that the weight average molecular weight thereof is equal to or greater than the lower limit described above because then the intermolecular interaction can be enhanced, and hence high mobility is obtained.

Examples of the polymer compound having a repeating structure include a π-conjugated polymer having a repeating structure in which the compound represented by Formula (1) represents at least one or more arylene groups or heteroarylene groups (thiophene or bithiophene), and a pendant-type polymer in which the compound represented by Formula (1) is bonded to the main chain of the polymer via the side chain. As the main chain of the polymer, polyacrylate, polyvinyl, polysiloxane, or the like is preferable, and as the side chain, an alkylene group, a polyethylene oxide group, or the like is preferable.

The compound represented by Formula (1) can be synthesized according to EP2251342A1, EP2301926A1, EP2301921A1, KR10-2012-0120886A, and Scheme 1 which will be described later.

In synthesizing the compound of the present invention, any of reaction conditions may be used. As a reaction solvent, any solvent may be used. Furthermore, in order to accelerate a ring forming reaction, an acid or a base is preferably used, and a base is particularly preferably used. The optimal reaction conditions vary with the structure of the intended compound.

<Intermediate Compound>

The synthetic intermediate having various substituents can be synthesized using known reactions in combination. Furthermore, various substituents may be introduced into the intermediate at any stage. After the intermediate is synthesized, it is preferable to purify the intermediate by column chromatography, recrystallization, or the like and then further purify it by sublimation. By the sublimation purification, it is possible to separate organic impurities and to effectively remove an inorganic salt, a residual solvent, and the like.

The present invention also relates to a compound represented by the following Formula (M1) and a compound represented by the following Formula (M2).

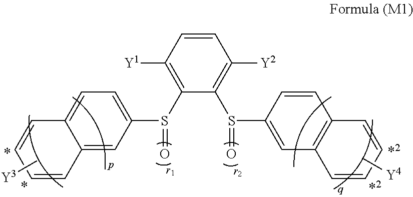

In Formula (M1),

each of Y¹ to Y⁴ independently represents a halogen atom, an alkoxy group, an acyloxy group, a hydroxy group, or a sulfonyl group,

each of * and *2 independently represents a position in which each of Y³ and Y⁴ can be substituted,

each of p and q independently represents an integer of 0 to 2, and

each of r1 and r2 independently represents an integer of 0 to 2.

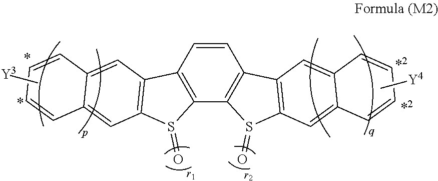

In Formula (M2),

each of Y³ and Y⁴ independently represents a halogen atom, an alkoxy group, an acyloxy group, a hydroxy group, or a sulfonyl group,

each of * and *2 independently represents a position in which each of Y³ and Y⁴ can be substituted,

each of p and q independently represents an integer of 0 to 2, and

each of r1 and r2 independently represents an integer of 0 to 2.

It is preferable that each of the compound represented by Formula (M1) described above and the compound represented by Formula (M2) described above is the intermediate compound of the compound represented by Formula (1) described above. The compound represented by Formula (1) described above can be synthesized according to Scheme 1, which will be described later, through a process in which the compound represented by Formula (M1) described above and the compound represented by Formula (M2) described above are synthesized as synthetic intermediate compounds.

In Formula (M1), each of Y¹ to Y⁴ is independently a halogen atom, an alkoxy group, an acyloxy group, a hydroxy group, or a sulfonyl group.

The halogen atom represented by Y¹ and Y² in Formula (M1) is preferably a chlorine atom, a bromine atom, or an iodine atom, more preferably a bromine atom or an iodine atom, and particularly preferably a bromine atom.

The halogen atom represented by Y³ and Y⁴ is preferably a chlorine atom, a bromine atom, or an iodine atom, more preferably a chlorine atom or a bromine atom, and particularly preferably a chlorine atom.

The alkoxy group represented by Y¹ to Y⁴ in Formula (M1) is preferably an alkoxy group having 1 to 5 carbon atoms, more preferably an alkoxy group having 1 to 3 carbon atoms, and particularly preferably a methoxy group.

The acyloxy group represented by Y¹ to Y⁴ in Formula (M1) is preferably an acyloxy group having 1 to 5 carbon atoms, more preferably an acyloxy group having 1 to 3 carbon atoms, and particularly preferably an acetyloxy group.

The sulfonyl group means a group represented by —SO₂R^{Tf}, and R^{Tf} represents a substituent. R^{Tf} that the sulfonyl group in Formula (M1) has is preferably a p-tolyl group or a substituted or unsubstituted alkyl group, more preferably a haloalkyl group, and particularly preferably a trifluoromethyl group. The sulfonyl group represented by Y¹ to Y⁴ in Formula (M1) is particularly preferably a trifluoromethanesulfonyl group.

The compound represented by Formula (M1) is preferably a compound in which each of Y¹ and Y² in the Formula (M1) is independently a bromine atom or an iodine atom, and each of Y³ and Y⁴ is independently a chlorine atom or an alkoxy group.

In Formula (M2), each of Y³ and Y⁴ independently represents a halogen atom, an alkoxy group, an acyloxy group, a hydroxy group, or a sulfonyl group.

The halogen atom represented by Y³ and Y⁴ in Formula (M2) is preferably a bromine atom or a chlorine atom, and more preferably a chlorine atom.

The alkoxy group represented by Y³ and Y⁴ in Formula (M2) is preferably an alkoxy group having 1 to 5 carbon atoms, more preferably an alkoxy group having 1 to 3 carbon atoms, and particularly preferably a methoxy group.

The acyloxy group represented by Y³ and Y⁴ in Formula (M2) is preferably an acyloxy group having 1 to 5 carbon atoms, more preferably an acyloxy group having 1 to 3 carbon atoms, and particularly preferably an acetyloxy group.

R^{Tf} that the sulfonyl group in Formula (M2) has is preferably a p-tolyl group or a substituted or unsubstituted alkyl group, more preferably a haloalkyl group, and particularly preferably a trifluoromethyl group. The sulfonyl group represented by Y³ and Y⁴ in Formula (M2) is particularly preferably a trifluoromethanesulfonyl group.

The compound represented by Formula (M2) is preferably a compound in which each of Y³ and Y⁴ in the Formula (M2) is independently a chlorine atom, an alkoxy group, a hydroxy group, or a sulfonyl group.

In Formulae (M1) and (M2), each of p and q independently represents an integer of 0 to 2. The preferred range of p and q in Formulae (M1) and (M2) is the same as the preferred range of p and q in Formula (1).

In Formulae (M1) and (M2), each of r1 and r2 independently represents an integer of 0 to 2. Each of r1 and r2 is preferably independently 0 or 1. More preferably, both of r1 and r2 are 0 or 1.

<Structure of Organic Transistor>

The organic transistor of the present invention has a semiconductor active layer containing the compound represented by Formula (1).

The organic transistor of the present invention may further have layers other than the semiconductor active layer.

The organic transistor of the present invention is preferably used as an organic field effect transistor (FET), and is more preferably used as an insulated gate-type FET in which the gate is insulated from channels.

Hereinafter, preferred structural aspects of the organic transistor of the present invention will be specifically described by using drawings, but the present invention is not limited to the aspects.

(Lamination Structure)

The lamination structure of an organic field effect transistor is not particularly limited, and various known structures can be adopted.

For example, the organic transistor of the present invention can adopt a structure (bottom gate/top contact type) in which an electrode, an insulator layer, a semiconductor active layer (organic semiconductor layer), and two electrodes are arranged in this order on the upper surface of a substrate which is a lowermost layer. In this structure, the electrode on the upper surface of the substrate as the lowermost layer is provided in a portion of the substrate, and the insulator layer is so disposed that it comes into contact with the substrate in a portion other than the electrode. The two electrodes provided on the upper surface of the semiconductor active layer are arranged in a state of being separated from each other.

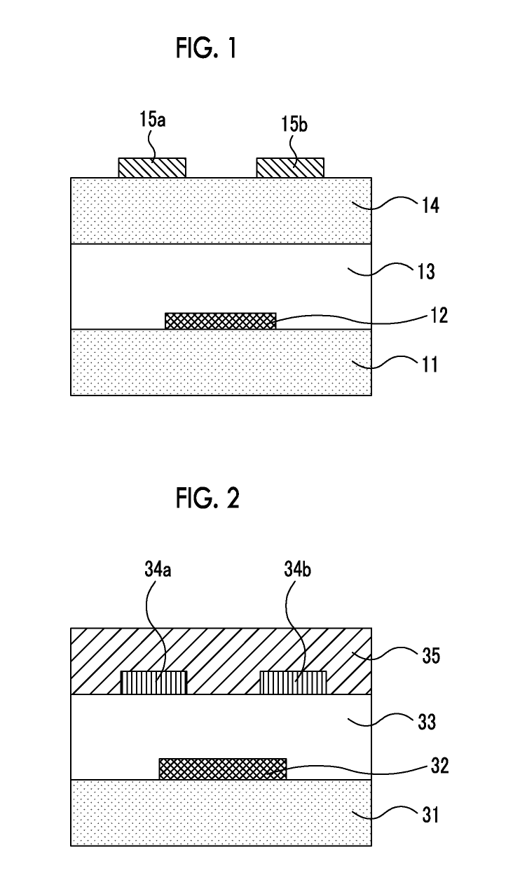

FIG. 1 shows the constitution of a bottom gate/top contact-type element. FIG. 1 is a schematic view showing a section of an exemplary structure of the organic transistor of the present invention. In the organic transistor shown in FIG. 1, a substrate 11 is disposed as a lowermost layer, an electrode 12 is provided in a portion of the upper surface thereof, and an insulator layer 13 is provided such that it covers the electrode 12 and comes into contact with the substrate 11 in a portion other than the electrode 12. On the upper surface of the insulator layer 13, a semiconductor active layer 14 is provided, and in a portion of the upper surface thereof, two electrodes 15a and 15b separated from each other are arranged.

In the organic transistor shown in FIG. 1, the electrode 12 is a gate, and the electrode 15a and the electrode 15b are a drain and a source respectively. The organic transistor shown in FIG. 1 is an insulated gate-type FET in which a channel as a path of electric currents between the drain and the source is insulated from the gate.

As an example of the structure of the organic transistor of the present invention, a bottom gate/bottom contact-type element can be exemplified.

FIG. 2 shows the constitution of the bottom gate/bottom contact-type element. FIG. 2 is a schematic view showing a section of the structure of an organic transistor manufactured as a substrate for measuring FET characteristics in examples of the present invention. In the organic transistor shown in FIG. 2, a substrate 31 is disposed as a lowermost layer, an electrode 32 is provided in a portion of the upper surface thereof, and an insulator layer 33 is provided such that it covers the electrode 32 and comes into contact with the substrate 31 in a portion other than the electrode 32. Furthermore, a semiconductor active layer 35 is provided on the upper surface of the insulator layer 33, and electrodes 34a and 34b are in a lower portion of the semiconductor active layer 35.

In the organic transistor shown in FIG. 2, the electrode 32 is a gate, and the electrode 34a and the electrode 34b are a drain and a source respectively. The organic transistor shown in FIG. 2 is an insulated gate-type FET in which a channel as a path of electric currents between the drain and the source is insulated from the gate.

As the structure of the organic transistor of the present invention, a top gate/top contact-type element in which an insulator and a gate electrode are in the upper portion of a semiconductor active layer or a top gate/bottom contact-type element can also be preferably used.

(Thickness)

In a case where the organic transistor of the present invention needs to be a thinner transistor, the total thickness of the transistor is preferably, for example, 0.1 μm to 0.5 μm.

(Sealing)

In order to improve the preservation stability of the organic transistor element by blocking the organic transistor element from the atmosphere or moisture, the entirety of the organic transistor element may be sealed with a metal sealing can, glass, an inorganic material such as silicon nitride, a polymer material such as parylene, a low-molecular weight material, or the like.

Hereinafter, preferred aspects of the respective layers of the organic transistor of the present invention will be described, but the present invention is not limited to the aspects.

<Substrate>

(Material)

The organic transistor of the present invention preferably includes a substrate.

The material of the substrate is not particularly limited, and known materials can be used. Examples of the material include a polyester film such as polyethylene naphthalate (PEN) or polyethylene terephthalate (PET), a cycloolefin polymer film, a polycarbonate film, a triacetylcellulose (TAC) film, a polyimide film, a material obtained by bonding these polymer films to extremely thin glass, ceramics, silicon, quartz, glass, and the like. Among these, silicon is preferable.

<Electrode>

(Material)

The organic transistor of the present invention preferably includes an electrode.

As the material constituting the electrode, known conductive materials such as a metal material like Cr, Al, Ta, Mo, Nb, Cu, Ag, Au, Pt, Pd, In, Ni, or Nd, an alloy material of these, a carbon material, and a conductive polymer can be used without particular limitation.

(Thickness)

The thickness of the electrode is not particularly limited, but is preferably 10 nm to 50 nm.

A gate width (or a channel width) W and a gate length (or a channel length) L are not particularly limited. However, a ratio of W/L is preferably equal to or greater than 10, and more preferably equal to or greater than 20.

<Insulating Layer>

(Material)

The material constituting the insulating layer is not particularly limited as long as an insulating effect is obtained as required. Examples of the material include silicon dioxide, silicon nitride, a fluorine polymer-based insulating material such as PTFE or CYTOP, a polyester insulating material, a polycarbonate insulating material, an acryl polymer-based insulating material, an epoxy resin-based insulating material, a polyimide insulating material, a polyvinyl phenol resin-based insulating material, a poly p-xylylene resin-based insulating material, and the like.

A surface treatment may be performed on the upper surface of the insulating layer. For example, it is possible to preferably use an insulating layer in which the silicon dioxide surface thereof is subjected to the surface treatment by being coated with hexamethyldisilazane (HMDS) or octadecyltrichlorosilane (OTS).

(Thickness)

The thickness of the insulating layer is not particularly limited. However, in a case where the film needs to be thinned, the thickness of the insulating layer is preferably 10 nm to 400 nm, more preferably 20 nm to 200 nm, and particularly preferably 50 nm to 200 nm.

<Semiconductor Active Layer>

(Material)

In the organic transistor of the present invention, the semiconductor active layer contains a compound represented by Formula (1), that is, the compound of the present invention.

The semiconductor active layer may be a layer consisting of the compound of the present invention or a layer further containing a polymer binder, which will be described later, in addition to the compound of the present invention. Furthermore, the semiconductor active layer may contain a residual solvent used at the time of forming a film.

The content of the polymer binder in the semiconductor active layer is not particularly limited. However, the content of the polymer binder is preferably within a range of 0% by mass to 95% by mass, more preferably within a range of 10% by mass to 90% by mass, even more preferably within a range of 20% by mass to 80% by mass, and particularly preferably within a range of 30% by mass to 70% by mass.

(Thickness)

The thickness of the semiconductor active layer is not particularly limited. However, in a case where the film needs to be thinned, the thickness of the semiconductor active layer is preferably 10 nm to 400 nm, more preferably 10 nm to 200 nm, and particularly preferably 10 nm to 100 nm.

[Organic Semiconductor Material for Non-Light-Emitting Organic Semiconductor Device]

The present invention also relates to an organic semiconductor material for a non-light-emitting organic semiconductor device containing the compound represented by Formula (1).

(Non-Light-Emitting Organic Semiconductor Device)

In the present specification, a “non-light-emitting organic semiconductor device” refers to a device which is not used for the purpose of emitting light. The non-light-emitting organic semiconductor device preferably uses an electronic element having a layered structure consisting of films. The non-light-emitting organic semiconductor device includes an organic transistor, an organic photoelectric conversion element (a solid-state imaging element used for a photosensor, a solar cell used for energy conversion, or the like), a gas sensor, an organic rectifying element, an organic inverter, an information recording element, and the like. The organic photoelectric conversion element can be used for both a photosensor (solid-state imaging element) and for energy conversion (a solar cell). Among these, an organic photoelectric conversion element and an organic transistor are preferable, and an organic transistor is more preferable. That is, the organic semiconductor material for a non-light-emitting organic semiconductor device of the present invention is preferably a material for an organic transistor as described above.

(Organic Semiconductor Material)

In the present specification, the “organic semiconductor material” is an organic material showing characteristics of a semiconductor. Just as the semiconductor composed of an inorganic material, the organic semiconductor is classified into a p-type (hole-transporting) organic semiconductor material conducting holes as carriers and an n-type (electron-transporting) organic semiconductor material conducting electrons as carriers.

The compound of the present invention may be used as any of the p-type organic semiconductor material and the n-type organic semiconductor material, but is preferably used as the p-type. The ease with which the carriers flow in the organic semiconductor is represented by a carrier mobility μ. The higher the carrier mobility μ, the better. The carrier mobility μ is preferably equal to or greater than 1×10⁻⁴ cm²/Vs, more preferably equal to or greater than 1×10⁻² cm²/Vs, particularly preferably equal to or greater than 5×10⁻² cm²/Vs, more particularly preferably equal to or greater than 1×10⁻¹ cm²/Vs, and even more particularly preferably equal to or greater than 2×10⁻¹ cm²/Vs. The carrier mobility can be determined by the characteristics of the prepared field effect transistor (FET) element or by a time-of-flight (TOF) measurement method.

[Organic Semiconductor Film for Non-Light-Emitting Organic Semiconductor Device]

(Material)

The present invention also relates to an organic semiconductor film for a non-light-emitting organic semiconductor device containing the compound represented by Formula (1).

As the organic semiconductor film for a non-light-emitting organic semiconductor device of the present invention, an aspect is also preferable in which the organic semiconductor film contains the compound represented by Formula (1) and does not contain a polymer binder.

Furthermore, the organic semiconductor film for a non-light-emitting organic semiconductor device of the present invention may contain the compound represented by Formula (1) and a polymer binder.

Examples of the polymer binder include an insulating polymer such as polystyrene, polycarbonate, polyarylate, polyester, polyamide, polyimide, polyurethane, polysiloxane, polysulfone, polymethyl methacrylate, polymethyl acrylate, cellulose, polyethylene, or polypropylene, a copolymer of these, a photoconductive polymer such as polyvinylcarbazole or polysilane, a conductive polymer such as polythiophene, polypyrrole, polyaniline, or poly p-phenylenevinylene, and a semiconductor polymer.

One kind of the aforementioned polymer binder may be used singly, or plural kinds thereof may be used concurrently.

The organic semiconductor material may be uniformly mixed with the polymer binder. Alternatively, the organic semiconductor material and the polymer binder may be totally or partially in a phase separation state. From the viewpoint of the charge mobility, a structure, in which the organic semiconductor and the binder are in a phase separation state along the film thickness direction in the film, is the most preferable because then the binder does not hinder the organic semiconductor from moving a charge.

Considering the mechanical strength of the film, a polymer binder having a high glass transition temperature is preferable. Furthermore, considering the charge mobility, a polymer binder having a structure not containing a polar group, a photoconductive polymer, and a conductive polymer are preferable.

The amount of the polymer binder used is not particularly limited. However, in the organic semiconductor film for a non-light-emitting organic semiconductor device of the present invention, the amount of the polymer binder used is preferably within a range of 0% by mass to 95% by mass, more preferably within a range of 10% by mass to 90% by mass, even more preferably within a range of 20% by mass to 80% by mass, and particularly preferably within a range of 30% by mass to 70% by mass.

In the present invention, by adopting the aforementioned structure as the structure of the compound, an organic film having excellent film quality can be obtained. Specifically, because the compound obtained in the present invention has excellent crystallinity, a sufficient film thickness can be obtained, and the obtained organic semiconductor film for a non-light-emitting organic semiconductor device of the present invention has excellent quality.

(Film Forming Method)

The compound of the present invention may be formed into a film on a substrate by any method.

At the time of forming the film, the substrate may be heated or cooled. By varying the temperature of the substrate, it is possible to control the film quality or the packing of molecules in the film. The temperature of the substrate is not particularly limited. However, it is preferably between 0° C. to 200° C., more preferably between 15° C. to 100° C., and particularly preferably between 20° C. to 95° C.

The compound of the present invention can be formed into a film on a substrate by a vacuum process or a solution process, and both of the processes are preferable.

Specific examples of the film forming method by a vacuum process include a physical vapor deposition method such as a vacuum vapor deposition method, a sputtering method, an ion plating method, or a molecular beam epitaxy (MBE) method and a chemical vapor deposition (CVD) method such as plasma polymerization, and it is particularly preferable to use a vacuum vapor deposition method.

Herein, the film forming method by a solution process refers to a method of dissolving an organic compound in a solvent which can dissolve the compound and forming a film by using the solution. Specifically, it is possible to use general methods like a coating method such as a casting method, a dip coating method, a die coater method, a roll coater method, a bar coater method, or a spin coating method, various printing methods such as an ink jet method, a screen printing method, a gravure printing method, a flexographic printing method, an offset printing method, or a micro-contact printing method, and a Langmuir-Blodgett (LB) method. It is particularly preferable to use a casting method, a spin coating method, an ink jet method, a gravure printing method, a flexographic printing method, an offset printing method, or a micro-contact printing method.

The organic semiconductor film for a non-light-emitting organic semiconductor device of the present invention is preferably prepared by a solution coating method. In a case where the organic semiconductor film for a non-light-emitting organic semiconductor device of the present invention contains a polymer binder, it is preferable to prepare a coating solution by dissolving or dispersing a material, which will be formed into a layer, and a polymer binder in an appropriate solvent and to form the organic semiconductor film by various coating methods.

Hereinafter, a coating solution for a non-light-emitting organic semiconductor device of the present invention that can be used for forming a film by a solution process will be described.

[Coating Solution for Non-Light-Emitting Organic Semiconductor Device]

The present invention also relates to a coating solution for a non-light-emitting organic semiconductor device that contains the compound represented by Formula (1).

In a case where a film is formed on a substrate by using a solution process, a material which will be formed into a layer is dissolved or dispersed in either or both of an appropriate organic solvent (for example, a hydrocarbon-based solvent such as hexane, octane, decane, toluene, xylene, mesitylene, ethylbenzene, decalin, or 1-methylnaphthalene, a ketone-based solvent such as acetone, methyl ethyl ketone, methyl isobutyl ketone, or cyclohexanone, a halogenated hydrocarbon-based solvent such as dichloromethane, chloroform, tetrachloromethane, dichloroethane, trichloroethane, tetrachloroethane, chlorobenzene, dichlorobenzene, or chlorotoluene, an ester-based solvent such as ethyl acetate, butyl acetate, or amyl acetate, an alcohol-based solvent such as methanol, propanol, butanol, pentanol, hexanol, cyclohexanol, methyl cellosolve, ethyl cellosolve, or ethylene glycol, an ether-based solvent such as dibutylether, tetrahydrofuran, dioxane, or anisole, an amide.imide-based solvent such as N,N-dimethylformamide, N,N-dimethylacetamide, 1-methyl-2-pyrrolidone, or 1-methyl-2-imidazolidinone, a sulfoxide-based solvent such as dimethyl sulfoxide, or a nitrile-based solvent such as acetonitrile) and water so as to obtain a coating solution, and a film can be formed by various coating methods by using the coating solution. One kind of the solvent may be used singly, or plural kinds thereof may be used in combination. Among these, a hydrocarbon-based solvent, a halogenated hydrocarbon-based solvent, and an ether-based solvent are preferable, toluene, xylene, mesitylene, tetralin, dichlorobenzene, and anisole are more preferable, and toluene, xylene, tetralin, and anisole are particularly preferable. The concentration of the compound represented by Formula (1) in the coating solution is preferably 0.1% by mass to 80% by mass, more preferably 0.1% by mass to 10% by mass, and particularly preferably 0.5% by mass to 10% by mass. In this way, a film having an arbitrary thickness can be formed.

In order to form a film by a solution process, the material needs to dissolve in the solvent exemplified above, but simply dissolving in a solvent is not good enough. Generally, even the material formed into a film by a vacuum process can dissolve in a solvent to some extent. The solution process includes a step of coating a substrate with a material by dissolving the material in a solvent and then forming a film by evaporating the solvent, and many of the materials not suitable for being formed into a film by the solution process have high crystallinity. Therefore, the material is inappropriately crystallized (aggregated) in the aforementioned step, and hence it is difficult to form an excellent film. The compound represented by Formula (1) is also excellent in the respect that it is not easily crystallized (aggregated).

As the coating solution for a non-light-emitting organic semiconductor device of the present invention, an aspect is also preferable in which the coating solution contains the compound represented by Formula (1) and does not contain a polymer binder.

Furthermore, the coating solution for a non-light-emitting organic semiconductor device of the present invention may contain the compound represented by Formula (1) and a polymer binder. In this case, a material, which will be formed into a layer, and a polymer binder are dissolved or dispersed in an appropriate solvent described above so as to prepare a coating solution, and by using the coating solution, a film can be formed by various coating methods. The polymer binder can be selected from those described above.

### EXAMPLES

Hereinafter, the characteristics of the present invention will be more specifically explained by describing examples and comparative examples. The materials, the amount thereof used, the proportion thereof, the content of treatment, the treatment procedure, and the like described in the following examples can be appropriately modified within a range that does not depart from the gist of the present invention. Accordingly, the scope of the present invention is not limited to the following specific examples.

### Examples 1 to 21 and Comparative Examples 1 to 6

### Synthesis Example

According to a specific synthesis procedure shown in the following Scheme 1, compound examples 3, 6, 10, 407, 20, 24, 298, 310, 326, 34, 62, 1, 16, 396, 386, 372, 358, 344, 416, 427, and 429 as compounds represented by Formula (1) and intermediate compounds (A) to (D) were synthesized.

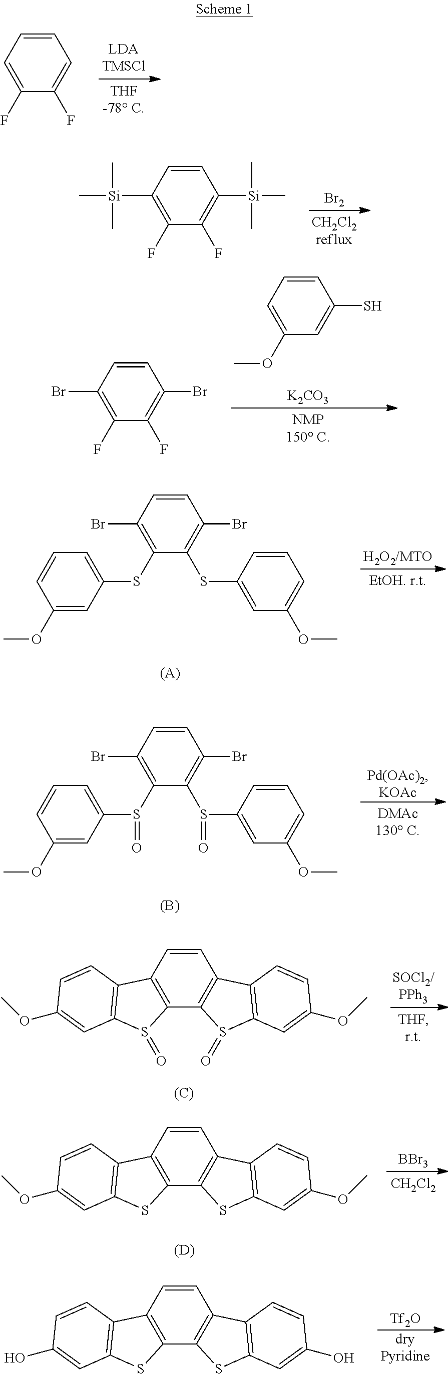

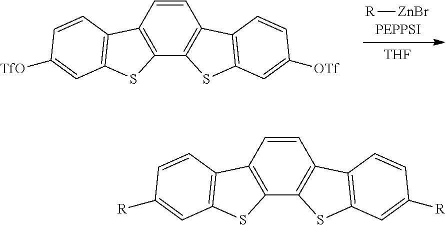

Synthesis of Intermediate Compound (A)

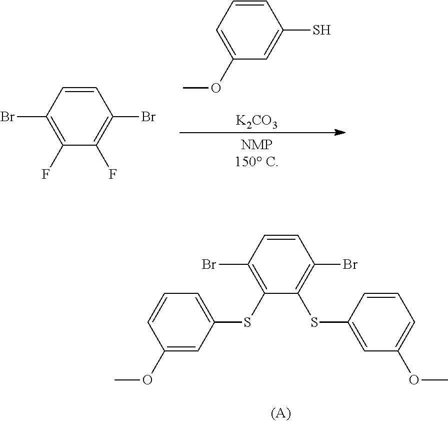

From 1,4-dibromo-2,3-difluorobenzene, an intermediate compound (A) was synthesized by the following procedure.

31 g of 1,4-dibromo-2,3-difluorobenzene, 37.9 g of potassium carbonate, and 31.9 g of 3-methoxyphenylthiol were added to 600 ml of N-methylpyrrolidone, followed by stirring for 2 hours at room temperature in a nitrogen atmosphere. The reaction mixture was filtered using celite, ethyl acetate and water were added to the filtrate for extraction, and oil layers were washed three times with dilute hydrochloric acid. Then, magnesium sulfate was added to the oil layers put together so as to dry the oil layers, followed by filtration, and ethyl acetate was distilled away under reduced pressure, thereby obtaining 45 g (yield: 77%) of an oil-like intermediate compound (A).

The structure of the intermediate compound (A) was identified by ¹H-NMR.

¹H-NMR (400 MHz, DMSO)

δ: 7.88 (s, 2H), 7.17 (t, 2H), 6.73 (d, 2H), 6.51 (s, 2H), 6.50 (d, 2H), 3.68 (s, 6H)

Synthesis of Intermediate Compound (B)

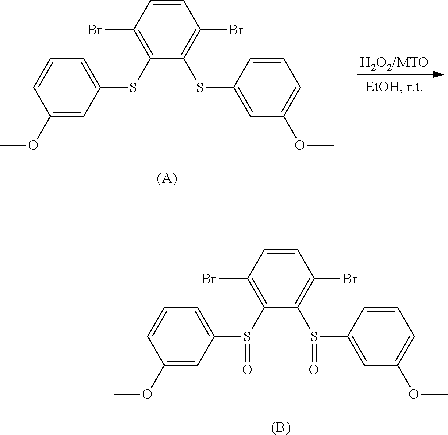

From the intermediate compound (A), an intermediate compound (B) was synthesized by the following procedure.

23.1 g of the intermediate compound (A) and 0.548 g of methyltrioxorhenium were dissolved in a mixed solvent of 176 ml of acetonitrile and 44 ml of dichloromethane, and 25.8 ml of 30% aqueous hydrogen peroxide was added dropwise thereto at room temperature. After the dropwise addition, the resulting mixture was stirred for 2 hours at room temperature, and the generated precipitate was collected by filtration, thereby obtaining 14.3 g (yield: 59%) of an intermediate compound (B) in the form of a white solid.

The structure of the intermediate compound (B) was identified by ¹H-NMR.

¹H-NMR (400 MHz, DMSO)

δ: 7.89 (s, 2H), 7.31 (t, 2H), 6.97 (d, 2H), 6.78 (s, 2H), 6.69 (d, 2H), 3.68 (s, 6H)

Synthesis of Intermediate Compound (C)

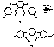

From the intermediate compound (B), an intermediate compound (C) was synthesized by the following procedure.

7.8 g of the intermediate (B), 96.5 mg of palladium acetate, and 2.8 g of potassium acetate were added to 86 ml of dimethyl acetamide, followed by vacuum deaeration, and then the resulting solution was heated and stirred for 2 hours at 130° C. in a nitrogen atmosphere. The reaction solution as cooled to room temperature and then poured into water, and the generated precipitate was collected by filtration. The solid separated by filtration was washed by ultrasonic dispersion in ethanol, thereby obtaining 4.2 g (yield: 76%) of an intermediate (C) in the form of a white solid.

The structure of the intermediate compound (C) was identified by ¹H-NMR.

¹H-NMR (400 MHz, CDCl₃)

δ: 7.85 (s, 2H), 7.75 (d, 2H), 7.59 (s, 2H), 7.17 (d, 2H), 3.94 (s, 6H)

Synthesis of Intermediate Compound (D)

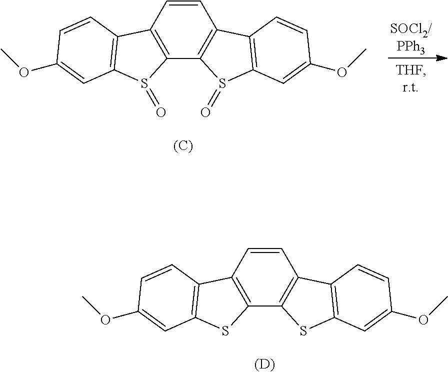

From the intermediate compound (C), an intermediate compound (D) was synthesized by the following procedure.

3.8 g of the intermediate compound (C), 5.63 g of potassium iodide, and 4.9 g of p-toluenesulfonic acid monohydrate were mixed together and irradiated with ultrasonic waves for 1 hour at 40° C. The reaction mixture was put into an aqueous sodium hydrogen sulfite solution, and the insoluble matter was collected by filtration. The solid separated by filtration was washed with toluene, thereby obtaining 2.7 g (yield: 78%) of an intermediate compound (D) in the form of a white solid.

The structure of the intermediate compound (D) was identified by ¹H-NMR.

¹H-NMR (400 MHz, CDCl₃)

δ: 8.10-8.07 (m, 4H), 7.40 (s, 2H), 7.10 (d, 2H), 3.94 (s, 6H)

Through the intermediate compound (D), compound examples 3, 6, 10, 407, 20, 24, 298, 310, 326, 34, 62, 1, 16, 396, 386, 368, 358, 344, 416, 427, and 429 as compounds represented by Formula (1) were synthesized. The obtained compounds were identified by elementary analysis, NMR, and MASS spectrometry.

The structures of comparative compounds 1 to 6 used in a semiconductor active layer (organic semiconductor layer) of comparative elements are shown below.

The comparative compound 1 is a compound described in Tetrahedron Lett., 1973 (17), 1561, and was synthesized according to the method described in the document.

The comparative compound 2 is a compound described in Rao, Tilak, Journal of Scientific and Industrial Research, 1958, vol. 17 B, pp. 260-265 and Kudo, Hirotaka; Tedjamulia, Marvin L.; Castle, Raymond N.; Lee, Milton L., Journal of Heterocyclic Chemistry, 1984, vol. 21, pp. 185-192, and was synthesized according to the method described in these documents.

The comparative compounds 3 and 4 are compounds 232 and 236 of EP2301926A1, and were synthesized according to the method described in the document.

The comparative compound 5 was synthesized according to the method described in EP2251342A1.

The comparative compound 6 is a compound 30 of KR2012-0120886A, and was synthesized according to the method described in the document.

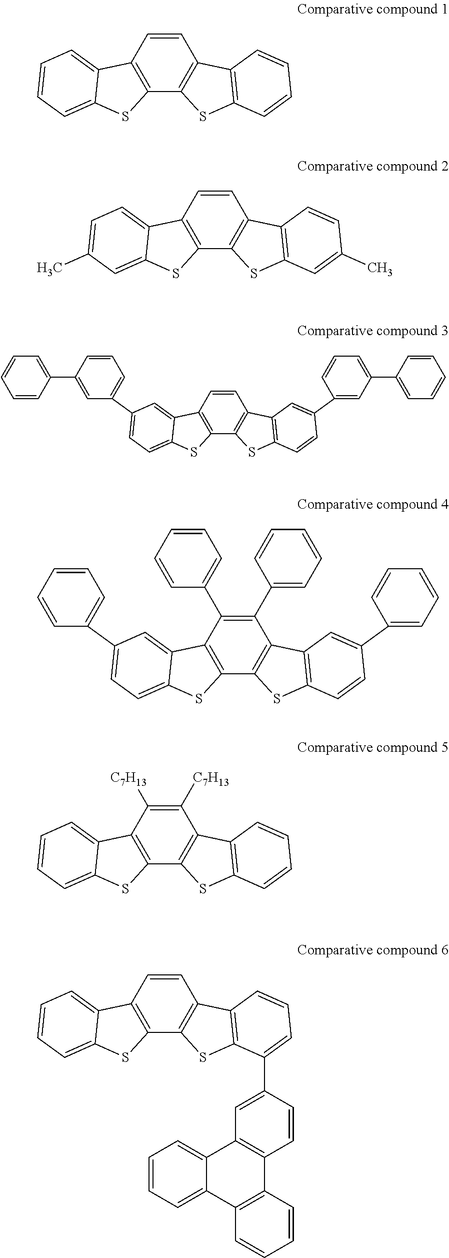

<Preparation/Evaluation of Element>

All of the materials used for preparing elements were purified by sublimation. Through high-performance liquid chromatography (TSKgel ODS-100Z manufactured by TOSOH CORPORATION), it was confirmed that the materials had purity (area ratio for absorption intensity at 254 nm) of equal to or higher than 99.5%.

<Formation of Semiconductor Active Layer (Organic Semiconductor Layer) by Using Compound Alone>

Each of the compounds of the present invention or the comparative compounds (1 mg each) was mixed with toluene (1 mL) and heated to 100° C., thereby obtaining a coating solution for a non-light-emitting organic semiconductor device. In a nitrogen atmosphere, the coating solution was cast onto a substrate for measuring FET characteristics that was heated to 90° C., thereby forming an organic semiconductor film for a non-light-emitting organic semiconductor device. In this way, an organic transistor element of Example 2 for measuring FET characteristics was obtained. As the substrate for measuring FET characteristics, a silicon substrate having a bottom gate/bottom contact structure was used which comprised chromium/gold (gate width W=100 mm, gate length L=100 μm) arranged to form a comb pattern as source and drain electrodes and comprised SiO₂ (film thickness: 200 nm) as an insulating layer (the structure is schematically shown in FIG. 2).

[Evaluation]

(a) Carrier Mobility

By using a semiconductor parameter analyzer (4156C manufactured by Agilent Technologies) connected to a semi-automatic prober (AX-2000 manufactured by Vector Semiconductor Co., Ltd.), the carrier mobility as one of the FET characteristics of the organic transistor element of each of the examples and comparative examples were evaluated under a normal pressure/nitrogen atmosphere.

Between the source electrode and the drain electrode of each organic film transistor element (FET element), a voltage of −80 V was applied, and the gate voltage was varied within a range of 20 V to −100 V. In this way, a carrier mobility μ was calculated using the following equation showing a drain current I_{d}.

I_{d}=(w/2L)μCᵢ(V_{g}−Vₜₕ)²

In the equation, L represents a gate length, W represents a gate width, Cᵢ represents a capacity of the insulating layer per unit area, V_{g} represents a gate voltage, and Vₜₕ represents a threshold voltage.

(b) Solubility

Each of the compounds of the present invention or comparative compounds (2% by mass, 1% by mass, 0.5% by mass, or 0.1% by mass each) was mixed with toluene (1 mL), heated to 100° C., and then left to stand for 30 minutes at room temperature. Thereafter, the concentration at which precipitation did not occur was determined, and the solubility in toluene was evaluated into 4 levels as below. For practical use, the compound needs to be evaluated to be A, B, C, or D. The compound preferably needs to be evaluated to be A, B, or C, more preferably needs to be evaluated to be A or B, and particularly preferably needs to be evaluated to be A.

A: Precipitation did not occur at 2% by mass.

B: Precipitation did not occur at 1% by mass but occurred at 2% by mass.

C: Precipitation did not occur at 0.5% by mass but occurred at 1% by mass.

D: Precipitation did not occur at 0.1% by mass but occurred at 0.5% by mass.

E: Precipitation occurred at 0.1% by mass.

(c) Threshold Voltage Shift after Repeated Driving (Threshold Shift)

Between the source electrode and the drain electrode of each organic transistor element (FET element), a voltage of −80 V was applied, and the element was repeatedly driven 100 times by varying the gate voltage within a range of +20 V to −100 V. In this way, the element was measured in the same manner as in the section (a), and a difference between a threshold voltage V_{before} before the repeated driving and a threshold voltage V_{after} after the repeated driving (|V_{after}−V_{before}|) was evaluated into 5 levels as below. The smaller the difference, the higher the stability of the element against repeated driving. Therefore, the smaller the difference, the more preferable.

|V_{after}−V_{before}|≦1 V A:

1 V<|V_{after}−V_{before}|≦5 V B:

5 V<|V_{after}−V_{before}|≦10 V C:

10 V<|V_{after}−V_{before}|≦15 V D:

|V_{after}−V_{before}|>15 V E:

(d) Quality of Coating Film

The quality of the coating film was evaluated by the following method.

For the semiconductor active layer of the organic transistor element of Example 2, a domain size was calculated within a range of 1 mm² by using a polarization microscope, and the average domain size was calculated. The obtained result was evaluated into 3 levels as below. There is no problem with the practical use of an organic transistor element evaluated to be D. However, the organic transistor element preferably needs to be evaluated to be A, B, or C, more preferably needs to be evaluated to be A or B, and particularly preferably needs to be evaluated to be A.

A: The average domain size was greater than 10 μm.

B: The average domain size was greater than 5 μm but equal to or less than 10 μm.

C: The average domain size was greater than 1 μm but equal to or less than 5 μm.

D: The average domain size was greater than 0.5 μm but equal to or less than 1 μm.

E: The average domain size was equal to or less than 0.5 μm.

(e) Variation

Each element was prepared by 30, the mobility of the prepared 30 elements was measured, and the coefficient of variation was calculated. The result was evaluated into 5 levels as below.

For practical use, an element evaluated to be C is also acceptable. However, the element preferably needs to be evaluated to be A or B, and more preferably needs to be evaluated to be A.

A: Less than 30%

B: Equal to or greater than 30% but less than 50%

C: Equal to or greater than 50% but less than 100%

D: Equal to or greater than 100% but less than 200%

E: Equal to or greater than 200%

The respective results are summarized in the following table.

TABLE 24

| | | | | Threshold | Quality of | |
|---|---|---|---|---|---|---|
| | Used compound | Mobility | Solubility | shift | coating film | Variation |
| Example 1 | Compound | 0.58 | A | A | A | A |
| | example 3 | | | | | |
| Example 2 | Compound | 0.77 | A | A | A | A |
| | example 6 | | | | | |
| Example 3 | Compound | 0.32 | A | A | A | A |
| | example 10 | | | | | |
| Example 4 | Compound | 0.58 | A | A | A | A |
| | example 407 | | | | | |
| Example 5 | Compound | 0.7 | B | A | A | A |
| | example 20 | | | | | |
| Example 6 | Compound | 1.1 | B | A | A | A |
| | example 24 | | | | | |
| Example 7 | Compound | 0.42 | B | B | A | A |
| | example 298 | | | | | |
| Example 8 | Compound | 0.28 | B | B | A | A |
| | example 310 | | | | | |
| Example 9 | Compound | 0.11 | B | B | A | A |
| | example 326 | | | | | |
| Example 10 | Compound | 0.13 | B | B | B | A |
| | example 34 | | | | | |
| Example 11 | Compound | 0.18 | B | B | B | A |
| | example 62 | | | | | |
| Example 12 | Compound | 0.13 | B | B | C | B |
| | example 1 | | | | | |
| Example 13 | Compound | 0.1 | B | B | C | B |
| | example 16 | | | | | |
| Example 14 | Compound | 0.01 | B | B | C | B |
| | example 396 | | | | | |
| Example 15 | Compound | 0.08 | B | C | C | C |
| | example 386 | | | | | |
| Example 16 | Compound | 0.05 | B | C | C | C |
| | example 372 | | | | | |
| Example 17 | Compound | 6.4 × 10⁻³ | B | C | C | C |
| | example 358 | | | | | |
| Example 18 | Compound | 8.6 × 10⁻³ | B | C | C | C |
| | example 344 | | | | | |
| Example 19 | Compound | 2.1 × 10⁻³ | C | C | C | C |
| | example 416 | | | | | |
| Example 20 | Compound | 2.4 × 10⁻³ | D | C | C | C |
| | example 427 | | | | | |
| Example 21 | Compound | 1.0 × 10⁻⁴ | B | C | D | C |
| | example 429 | | | | | |
| Comparative | Comparative | 6.8 × 10⁻⁵ | E | C | E | E |
| example 1 | compound 1 | | | | | |
| Comparative | Comparative | 3.7 × 10⁻⁴ | E | C | E | E |
| example 2 | compound 2 | | | | | |
| Comparative | Comparative | No | E | C | C | D |
| example 3 | compound 3 | characteristics | | | | |
| Comparative | Comparative | No | E | C | D | D |
| example 4 | compound 4 | characteristics | | | | |
| Comparative | Comparative | No | D | C | D | D |
| example 5 | compound 5 | characteristics | | | | |
| Comparative | Comparative | No | E | D | E | E |
| example 6 | compound 6 | characteristics | | | | |

From the results shown in the above table, it was understood that the compound of the present invention exhibits excellent solubility in an organic solvent, and the organic transistor element using the compound of the present invention has high carrier mobility. Therefore, it was understood that the compound of the present invention can be preferably used as an organic semiconductor material for a non-light-emitting organic semiconductor device.

Particularly, Examples 1 to 6 using a compound represented by Formula (1), in which X is a S atom, each of R² and R⁹ is an alkyl group, and each of R¹, R³ to R⁸, and R¹⁰ is a hydrogen atom, showed excellent results in each of the carrier mobility, solubility, threshold voltage shift (threshold shift) after repeated driving, quality of coating film, and variation.

In contrast, the organic transistor elements using the comparative compounds 3 to 6 had poor carrier mobility that could not be evaluated. Furthermore, the comparative compounds 1 to 4 and 6 exhibited poor solubility in an organic solvent.

The organic transistor element using the compound of the present invention showed only a slight threshold voltage shift (threshold shift) after repeated driving, was excellent in the quality of the coating film, showed a small variation, and had excellent driving stability.

### Examples 101 to 121 and Comparative Examples 101 to 106

### Formation of Semiconductor Active Layer (Organic Semiconductor Layer) by Using Compound and Binder Together

Each of the compounds of the present invention or the comparative compounds (1 mg each) was mixed with 1 mg of PαMS (poly(α-methylstyrene, Mw=300,000), manufactured by Sigma-Aldrich Co. LLC.) and toluene (1 mL), and the mixture was heated to 100° C., thereby obtaining a coating solution. Organic transistor elements of Examples 101 to 121 and Comparative examples 101 to 106 for measuring FET characteristics were prepared in the same manner as in Examples 1 to 21 and Comparative examples 1 to 6 except that the coating solution obtained as above was used, and evaluated in the same manner as in Examples 1 to 21 and Comparative examples 1 to 6.

As a result, it was understood that the organic transistor elements show the same trend as in Examples 1 to 21 and Comparative examples 1 to 6.

### Examples 201 to 221 and Comparative Examples 201 to 206

### Formation of Semiconductor Active Layer (Organic Semiconductor Layer)

The surface of a silicon wafer, which comprised SiO₂ (film thickness: 370 nm) as a gate insulating film, was treated with octyltrichlorosilane.

Each of the compounds of the present invention or the comparative compounds (1 mg each) was mixed with toluene (1 mL), and the mixture was heated to 100° C., thereby preparing a coating solution for a non-light-emitting organic semiconductor device. In a nitrogen atmosphere, the coating solution was cast onto the silicon wafer which had been heated to 90° C. and undergone surface treatment with octylsilane, thereby forming an organic semiconductor film for a non-light-emitting organic semiconductor device.

Furthermore, gold was deposited onto the surface of the film by using a mask so as to prepare source and drain electrodes, thereby obtaining organic transistor elements of Examples 201 to 221 and Comparative examples 201 to 206 having a bottom gate/top contact structure with a gate width W=5 mm and a gate length L=80 μm (the structure is schematically shown in FIG. 1).

By using a semiconductor parameter analyzer (4156C manufactured by Agilent Technologies) connected to a semi-automatic prober (AX-2000 manufactured by Vector Semiconductor Co., Ltd.), the FET characteristics of the organic transistor elements of Examples 201 to 221 and Comparative examples 201 to 206 were evaluated in the same manner as in Examples 1 to 21 and Comparative examples 1 to 6 under a normal pressure/nitrogen atmosphere.

As a result, it was understood that the organic transistor elements show the same trend as in Examples 1 to 21 and Comparative examples 1 to 6.

### EXPLANATION OF REFERENCES

11: substrate
12: electrode
13: insulator layer
14: semiconductor active layer (organic substance layer, organic semiconductor layer)
15a, 15b: electrode
31: substrate
32: electrode
33: insulator layer
34a, 34b: electrode
35: semiconductor active layer (organic substance layer, organic semiconductor layer)

What is claimed is:

1. An organic transistor comprising a compound represented by the following Formula (1) in a semiconductor active layer; 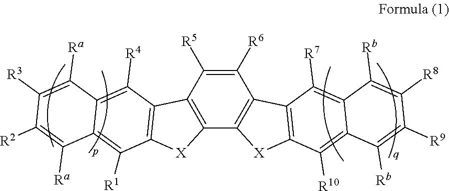
in Formula (1),
each X independently represents an O atom, a S atom, or a Se atom,
each of p and q independently represents an integer of 0 to 2,
each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, or R^{b} is a substituent represented by Formula (W), and in a case where at least one of R⁵ or R⁶ is a substituent represented by Formula (W), L in Formula (W) represented by R⁵ and R⁶ is a divalent linking group represented by the following Formula (L-2) or (L-3);
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 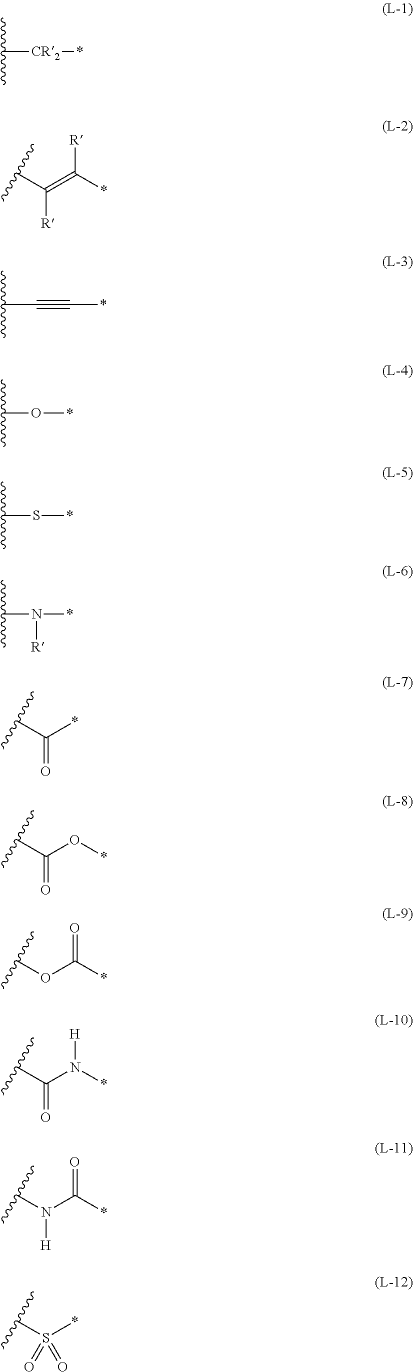 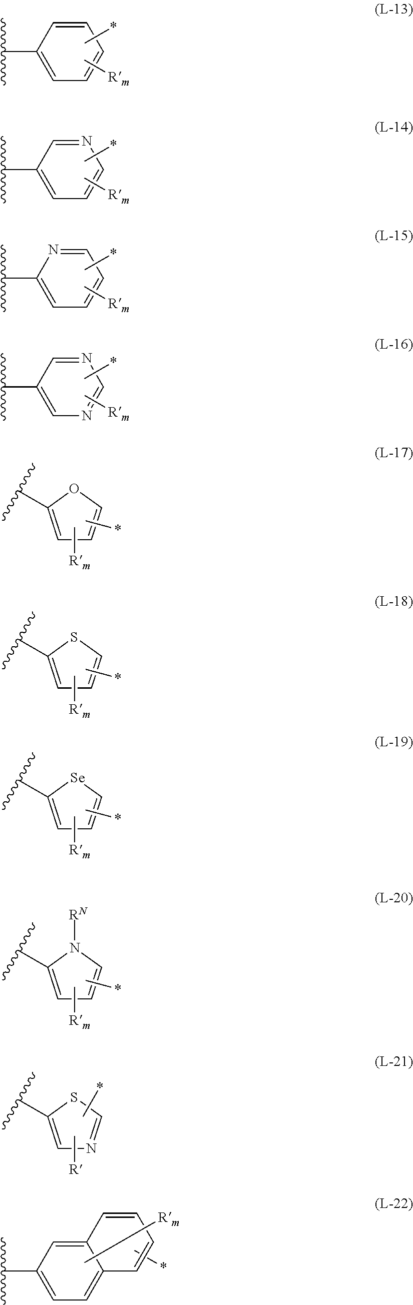 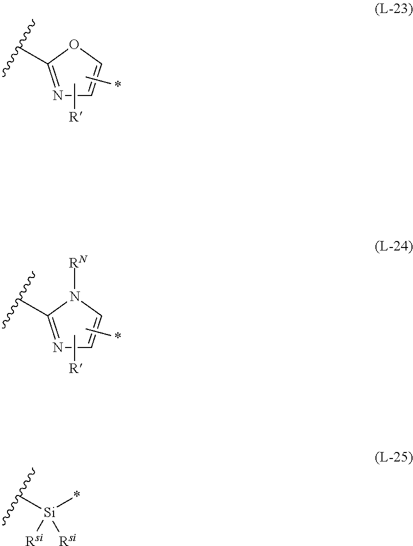
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.

2. The organic transistor according to claim 1,
wherein in the Formula (1), each of R¹, R⁴ to R⁷, R¹⁰, R^{a}, and R^{b} is independently a hydrogen atom or a halogen atom,
each of R², R³, R⁸, and R⁹ is independently a hydrogen atom, a halogen atom, or a substituent represented by the Formula (W), and at least one of R², R³, R⁸, or R⁹ is a substituent represented by the Formula (W).

3. The organic transistor according to claim 1,
wherein in the Formula (1), each of p and q is independently 0 or 1.

4. The organic transistor according to claim 1,
wherein in the Formula (1), p and q satisfy p=q=0 or p=q=1.

5. The organic transistor according to claim 1,
wherein the compound represented by the Formula (1) is a compound represented by the following Formula (2-1); 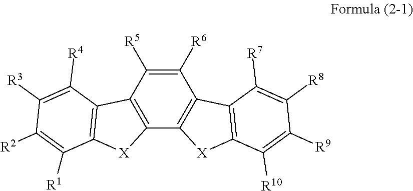
in Formula (2-1),
each X independently represents an O atom, a S atom, or a Se atom,
each of R¹, R³ to R⁸, and R¹⁰ independently represents a hydrogen atom or a halogen atom,
each of R² and R⁹ is independently a hydrogen atom, a halogen atom, or a substituent represented by the following Formula (W), at least one of R² or R⁹ is a substituent represented by Formula (W), and in a case where a substituent represented by Formula (W) is an alkyl group, the substituent represented by Formula (W) is limited to a linear alkyl group having 4 to 18 carbon atoms or a branched alkyl group having 4 or more carbon atoms;
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 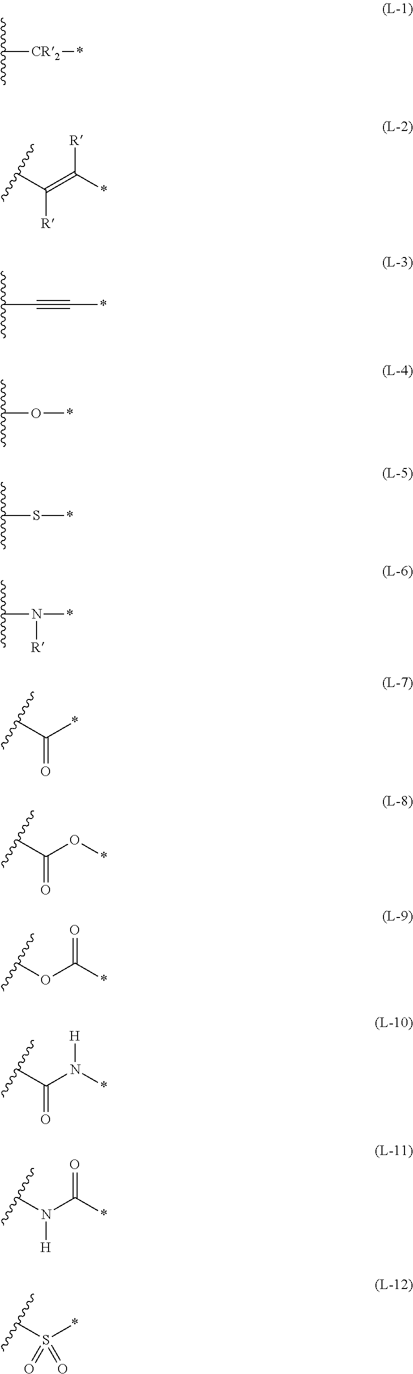 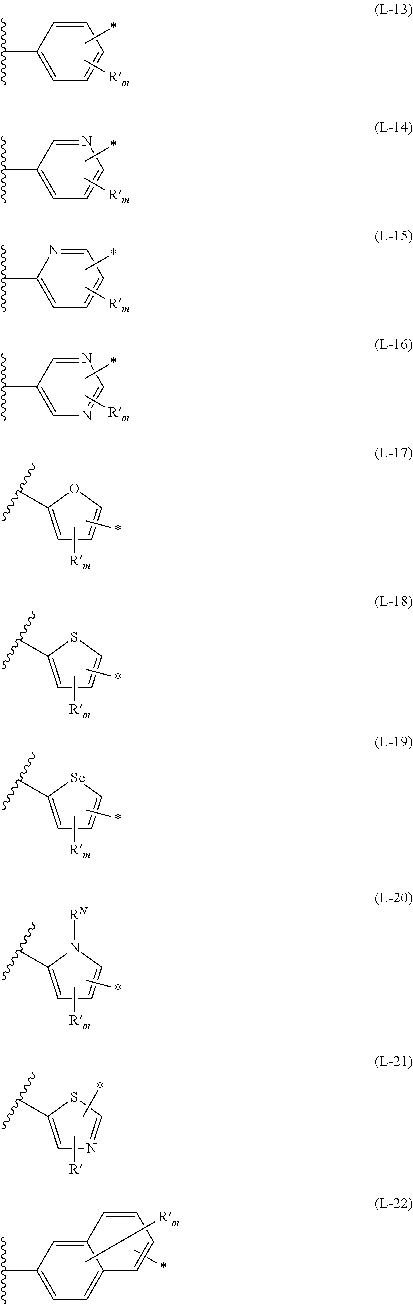 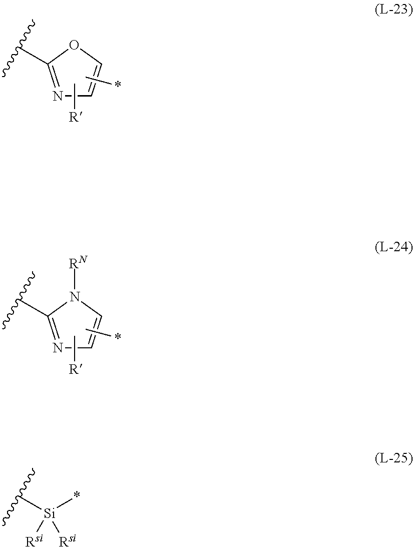
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.

6. The organic transistor according to claim 1,
wherein the compound represented by the Formula (1) is a compound represented by the following Formula (2-2); 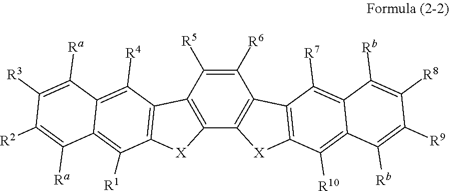
in Formula (2-2),
each X independently represents an O atom, a S atom, or a Se atom,
each of R¹, R⁴ to R⁷, R¹⁰, R^{a}, and R^{b} is independently a hydrogen atom or a halogen atom,
each of R², R³, R⁸, and R⁹ independently represents a hydrogen atom, a halogen atom, or a substituent represented by the following Formula (W), and at least one of R², R³, R⁸, or R⁹ represents a substituent represented by Formula (W);
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 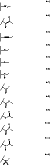 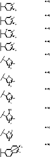 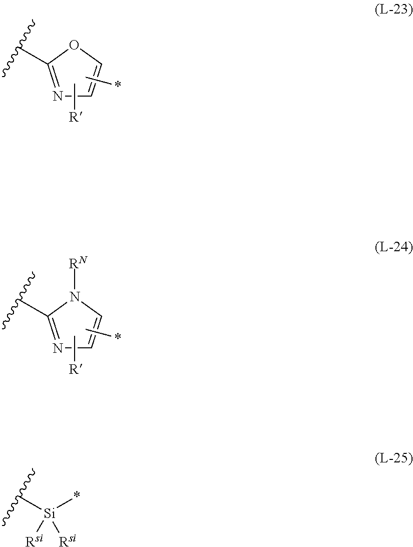
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.

7. The organic transistor according to claim 1,
wherein in the Formula (1), X is a S atom.

8. The organic transistor according to claim 1,
wherein in the Formula (1), both of R² and R⁹ are a substituent represented by the Formula (W), and both of R³ and R⁸ are a hydrogen atom or a halogen atom, or
both of R³ and R⁸ are a substituent represented by the Formula (W), and both of R² and R⁹ are a hydrogen atom or a halogen atom.

9. The organic transistor according to claim 1,
wherein both of R² and R⁹ are a substituent represented by the Formula (W), and both of R³ and R⁸ are a hydrogen atom.

10. The organic transistor according to claim 1,
wherein the total number of carbon atoms contained in a group represented by the Formula (W) is 4 to 18.

11. The organic transistor according to claim 1,
wherein in the Formula (W), L is a divalent linking group represented by any one of the Formulae (L-1) to (L-4) or a divalent linking group in which a divalent linking group represented by any one of the Formulae (L-2) to (L-4) is bonded to a divalent linking group represented by the Formula (L-1).

12. The organic transistor according to claim 1,
wherein in the Formula (W), L is a divalent linking group represented by the Formula (L-1), and R is a substituted or unsubstituted alkyl group.

13. A compound represented by the following Formula (2-1); 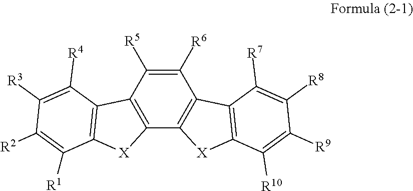
in Formula (2-1),
each X independently represents an O atom, a S atom, or a Se atom,
each of R¹, R³ to R⁸, and R¹⁰ independently represents a hydrogen atom or a halogen atom,
each of R² and R⁹ is independently a hydrogen atom, a halogen atom, or a substituent represented by the following Formula (W), at least one of R² or R⁹ is a substituent represented by Formula (W), and in a case where a substituent represented by Formula (W) is an alkyl group, the substituent represented by Formula (W) is limited to a linear alkyl group having 4 to 18 carbon atoms or a branched alkyl group having 4 or more carbon atoms;
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 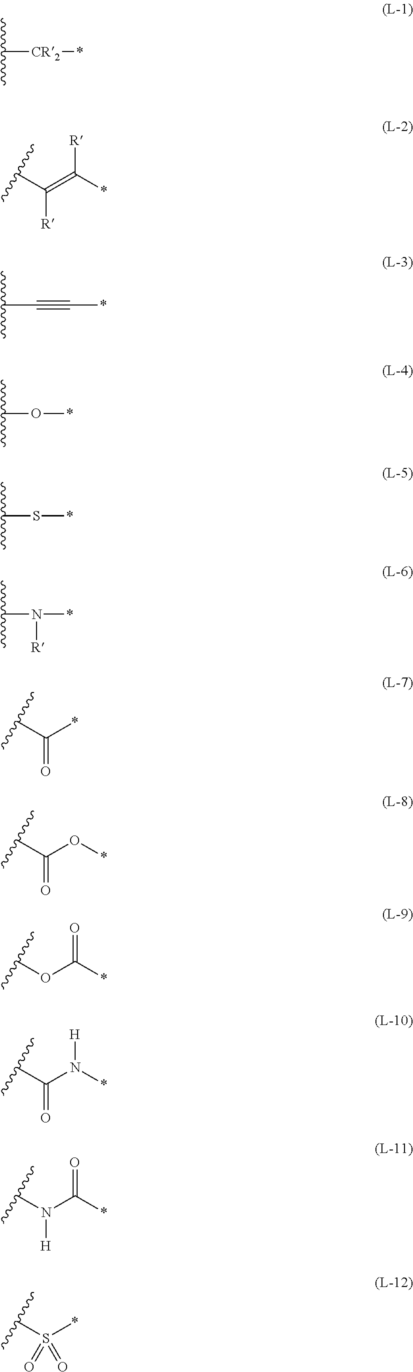 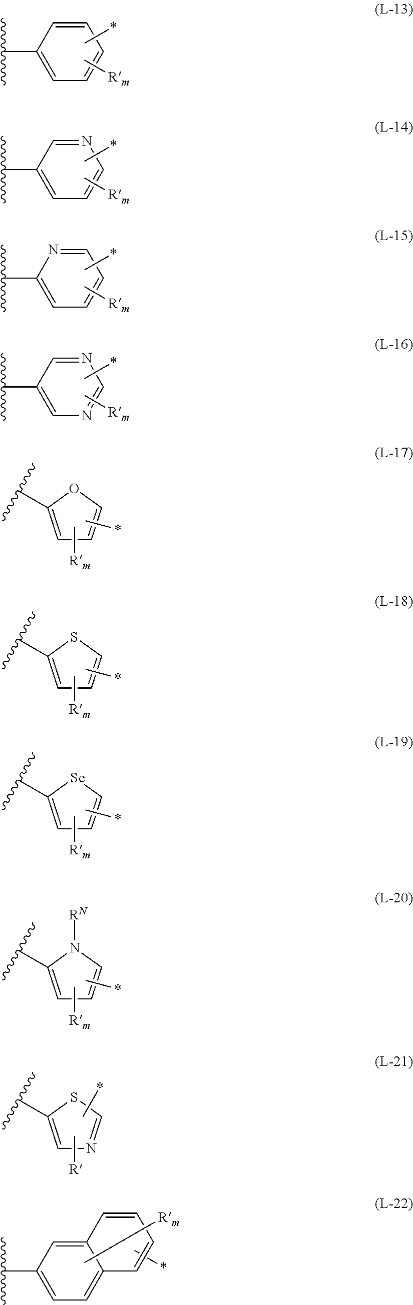 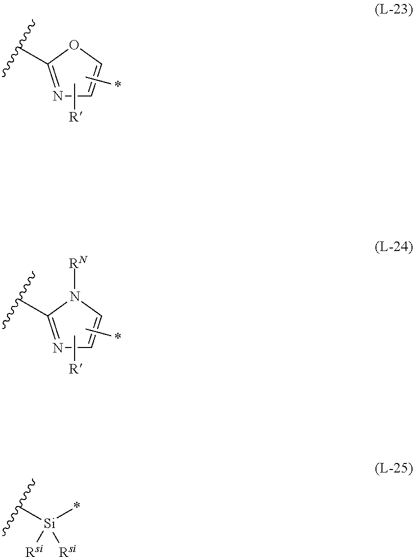
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.

14. A compound represented by the following Formula (2-2); 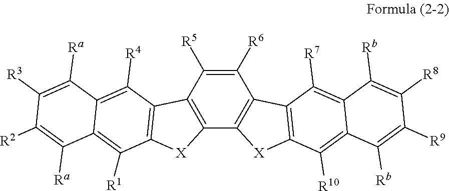
in Formula (2-2),
each X independently represents an O atom, a S atom, or a Se atom,
each of R¹, R⁴ to R⁷, R¹⁰, R^{a}, and R^{b} is independently a hydrogen atom or a halogen atom,
each of R², R³, R⁸, and R⁹ independently represents a hydrogen atom, a halogen atom, or a substituent represented by the following Formula (W), and at least one of R², R³, R⁸, or R⁹ represents a substituent represented by Formula (W),
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 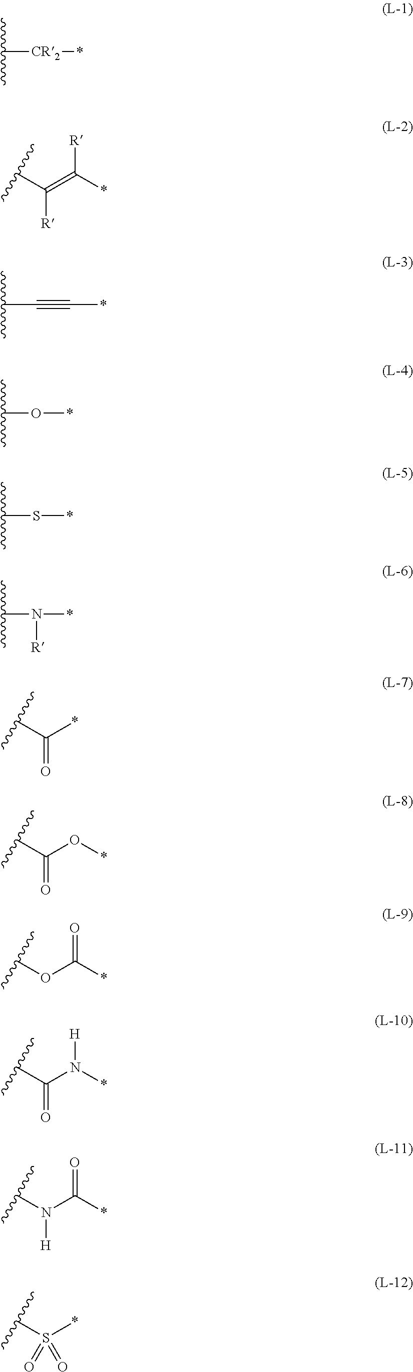 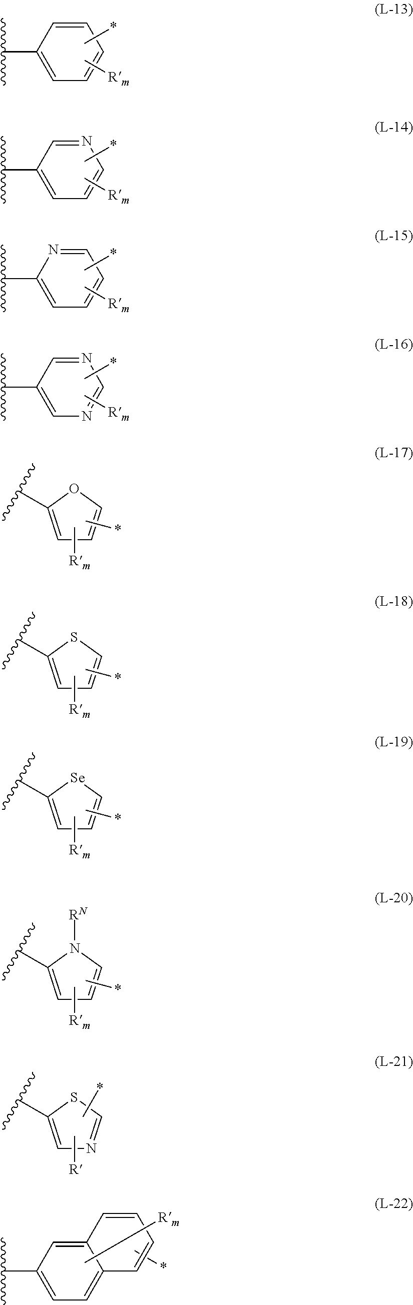 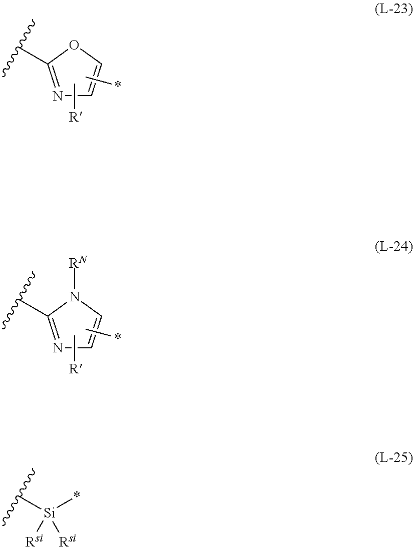
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.

15. The compound according to claim 13,
wherein in the Formula (2-1), X is a S atom.

16. The compound according to claim 13,
wherein in the Formula (2-1), both of R² and R⁹ are a substituent represented by the Formula (W), and both of R³ and R⁸ are a hydrogen atom or a halogen atom, or
both of R³ and R⁸ are a substituent represented by the Formula (W), and both of R² and R⁹ are a hydrogen atom or a halogen atom.

17. The compound according to claim 13,
wherein both of R² and R⁹ are a substituent represented by the Formula (W), and both of R³ and R⁸ are a hydrogen atom.

18. The compound according to claim 13,
wherein the total number of carbon atoms contained in a group represented by the Formula (W) is 4 to 18.

19. The compound according to claim 13,
wherein in the Formula (W), L is a divalent linking group represented by any one of the Formulae (L-1) to (L-4) or a divalent linking group in which a divalent linking group represented by any one of the Formulae (L-2) to (L-4) is bonded to a divalent linking group represented by the Formula (L-1).

20. The compound according to claim 13,
wherein in the Formula (W), L is a divalent linking group represented by the Formula (L-1), and R is a substituted or unsubstituted alkyl group.

21. An organic semiconductor material for a non-light-emitting organic semiconductor device, comprising the compound represented by the Formula (1) according to claim 1.

22. A material for an organic transistor, comprising the compound represented by the Formula (1) according to claim 1.

23. A coating solution for a non-light-emitting organic semiconductor device, comprising the compound represented by the Formula (1) according to claim 1.

24. A coating solution for a non-light-emitting organic semiconductor device according to claim 23, further comprising a polymer binder.

25. An organic semiconductor film for a non-light-emitting organic semiconductor device, comprising the compound represented by the Formula (1) according to claim 1.

26. An organic semiconductor film for a non-light-emitting organic semiconductor device according to claim 25, further comprising a polymer binder.

27. The organic semiconductor film for a non-light-emitting organic semiconductor device according to claim 25 that is prepared by a solution coating method.

28. A compound represented by the following Formula (M1); 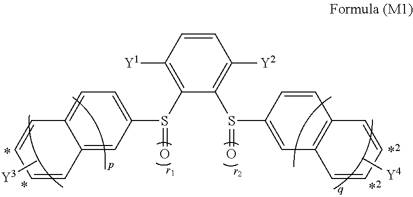
in Formula (M1),
each of Y¹ to Y⁴ independently represents a halogen atom, an alkoxy group, an acyloxy group, a hydroxy group, or a sulfonyl group,
each of * and *2 independently represents a position in which each of Y³ and Y⁴ can be substituted,
each of p and q independently represents an integer of 0 to 2, and
each of r1 and r2 independently represents an integer of 0 to 2.

29. A compound represented by the following Formula (M2); 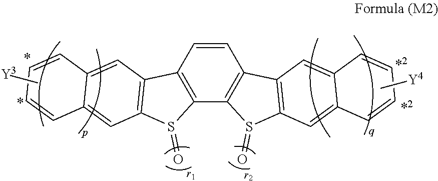
in Formula (M2),
each of Y³ and Y⁴ independently represents a halogen atom, an alkoxy group, an acyloxy group, a hydroxy group, or a sulfonyl group,
each of * and *2 independently represents a position in which each of Y³ and Y⁴ can be substituted,
each of p and q independently represents an integer of 0 to 2, and
each of r1 and r2 independently represents an integer of 0 to 2.

30. The compound according to claim 28 that is an intermediate compound of the compound represented by the following Formula (1): 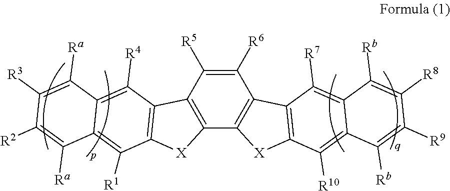
in Formula (1),
each X independently represents an O atom, a S atom, or a Se atom,
each of p and q independently represents an integer of 0 to 2,
each of R¹ to R¹⁰, R^{a}, and R^{b} independently represents a hydrogen atom, a halogen atom, or a substituent represented by the following Formula (W), at least one of R¹, R², R³, R⁴, R⁵, R⁶, R⁷, R⁸, R⁹, R¹⁰, R^{a}, or R^{b} is a substituent represented by Formula (W), and in a case where at least one of R⁵ or R⁶ is a substituent represented by Formula (W), L in Formula (W) represented by R⁵ and R⁶ is a divalent linking group represented by the following Formula (L-2) or (L-3);
-L-R Formula (W)
in Formula (W),
L represents a divalent linking group represented by any one of the following Formulae (L-1) to (L-25) or a divalent linking group in which two or more divalent linking groups represented by any one of the following Formulae (L-1) to (L-25) are bonded to each other, and
R represents a substituted or unsubstituted alkyl group, a cyano group, a vinyl group, an ethynyl group, an oxyethylene group, an oligo-oxyethylene group in which a repetition number v of an oxyethylene unit is equal to or greater than 2, a siloxane group, an oligosiloxane group having two or more silicon atoms, or a substituted or unsubstituted trialkylsilyl group; 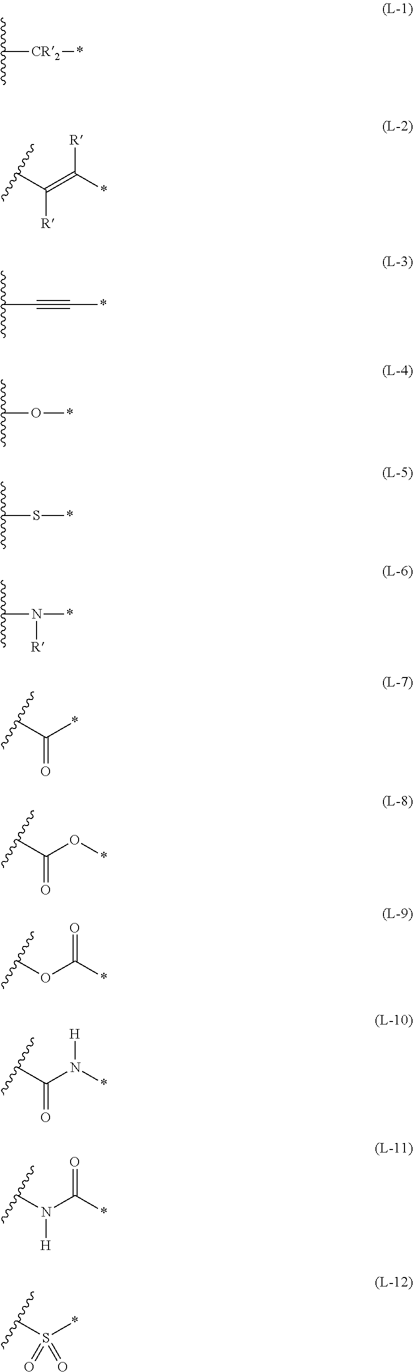 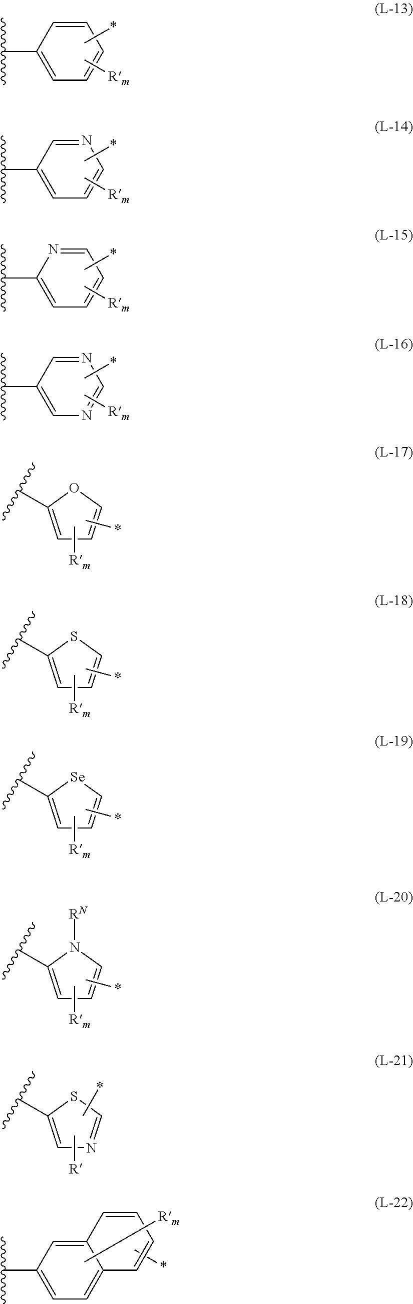 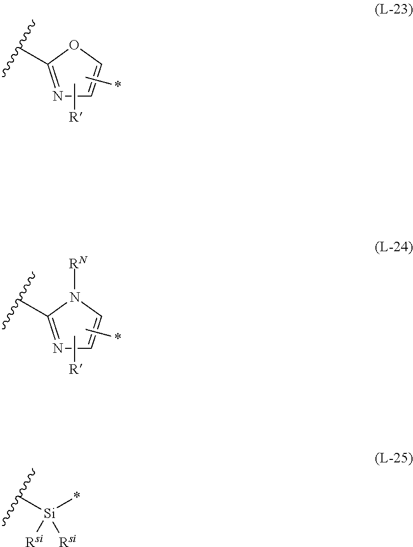
in Formulae (L-1) to (L-25),
the portion of a wavy line represents a position of bonding to a condensed cyclic skeleton,
m in Formula (L-13) represents 4, m in Formulae (L-14) and (L-15) represents 3, m in Formulae (L-16) to (L-20) represents 2, m in Formula (L-22) represents 6,
each R′ in Formulae (L-1), (L-2), (L-6), and (L-13) to (L-24) independently represents a hydrogen atom or a substituent,
R^{N} represents a hydrogen atom or a substituent, and
each R^{si} independently represents a hydrogen atom, an alkyl group, an alkenyl group, or an alkynyl group.
